(19) United States
(12) Patent Application Publication
(10) Pub. No.: US 2026/0218309 A1
(43) Pub. Date: Jul. 30, 2026

(54) NUCLEIC ACID PROBES

(71) Applicant: Arima Genomics, Inc., Carlsbad, CA (US)

(72) Inventors: Jon-Matthew Belton, San Diego, CA (US); Anthony Schmitt, Holly Springs, NC (US); Kristin Sikkink, San Diego, CA (US); Siddarth Selvaraj, San Marcos, CA (US); Christopher Roberts, San Diego, CA (US)

(73) Assignee: Arima Genomics, Inc., Carlsbad, CA (US)

(21) Appl. No.: 19/147,284

(22) PCT Filed: Jan. 10, 2024
(86) PCT No.: PCT/US2024/011120

§ 371 Date: Jul. 10, 2025

Related U.S. Application Data
(60) Provisional application No. 63/438,512, filed on Jan. 11, 2023.

(51) Int. Cl. C12Q 1/6886; C12Q 1/6806

(52) U.S. Cl. CPC C12Q 1/6886; C12Q 1/6806; C12Q 2600/156

(57) ABSTRACT

The technology relates in part to compositions that include oligonucleotide probes useful for enriching nucleic acid preparation products for analysis, kits and related methods. The technology further relates in part to Chromosome conformation capture (3C) and similar technologies. The technology further relates in part to kits, compositions, and methods involving oligonucleotide probes capable of interacting with cancer genes.

### CROSS REFERENCE TO RELATED APPLICATIONS

This application claims the benefit under 35 U.S.C. § 119(e) of U.S. provisional application No. 63/438,512, filed Jan. 11, 2023, the entire contents of which are incorporated by reference herein.

### FIELD

The technology relates in part to compositions that include oligonucleotide probes useful for enriching nucleic acid preparation products for analysis, kits, and related methods.

### BACKGROUND

Chromosome conformation capture (3C) and similar technologies (HiC, 4C, 5C and the like) have been used to map long-range interactions and which probes the three-dimensional architecture of genomes.

However, there are some limitations with these technologies, including that there is a need to sequence very deep and thus the technology is time consuming as well as expensive and there is a need of developing new techniques that can solve those problems and enable the possibility to evaluate and detect direct intra- and inter-chromosomal interactions of interest, and potentially to utilize the information to diagnose specific medical and/or biological conditions.

Sahlén et al. (WO2014168575) discloses a method (“capture Hi-C”) of targeted chromosome conformation capture which combines HiC technology with hybridization of probes to targeted regions which allows for enrichment of the targeted regions for HiC analysis.

Disclosed herein is a composition of nucleic acid probes for use in capture-C technologies and methods associated therewith.

### SUMMARY

Described herein are kits comprising: a plurality of oligonucleotide probes capable of hybridizing to target nucleic acid fragments of cancer gene exons, introns, promoters and external regions surrounding cancer genes, resulting from fragmentation of a nucleic acid sample, wherein: a plurality of oligonucleotide probes capable of hybridizing to external regions comprises (i) a set of probe pairs each capable of hybridizing to a target nucleic acid fragment of about 260 consecutive nucleotides or longer, and (ii) a set of single oligonucleotide probes each capable of hybridizing to a nucleic acid fragment of about 130 consecutive nucleotides to about 260 consecutive nucleotides in length; the probes in the set of oligonucleotide probe pairs and the set of single oligonucleotide probes comprise a polynucleotide (i) 120 consecutive nucleotides in length, (ii) 100% complementary to a subsequence of a nucleic acid fragment, (iii) containing an average GC content of about 40 percent to about 60 percent, and (iv) complementary to a subsequence of a nucleic acid that does not repeat in the nucleic acid fragments; each of the probe pairs in the set of probe pairs comprises (i) a first oligonucleotide probe comprising a 5′ end 5 consecutive nucleotides from the 5′ end of the fragment, and (ii) a second oligonucleotide probe comprising a 3′ end 5 consecutive nucleotides from the 3′ end of the nucleic acid fragment to which the first oligonucleotide probe of the probe pair is capable of hybridizing; and each probe in the set of single oligonucleotide probes comprises a 5′ or 3′ end 5 consecutive nucleotides from the 5′ end of a nucleic acid fragment.

Also described herein are compositions comprising: a plurality of oligonucleotide probes capable of hybridizing to target nucleic acid fragments of cancer gene exons, introns, promoters, and external regions surrounding cancer genes resulting from nucleic acid fragmentation of a nucleic acid sample, wherein: a plurality of external region directed oligonucleotide probes capable of hybridizing to target nucleic acid fragments from external regions comprises (i) a set of probe pairs each capable of hybridizing to a target nucleic acid fragment of about 260 consecutive nucleotides or longer, and (ii) a set of single oligonucleotide probes each capable of hybridizing to a nucleic acid fragment of about 130 consecutive nucleotides to about 260 consecutive nucleotides in length; the external region directed oligonucleotide probes in the set of oligonucleotide probe pairs and the set of single oligonucleotide probes comprise a polynucleotide (i) about 110 to about 130 consecutive nucleotides in length, (ii) substantially complementary to a subsequence of a nucleic acid fragment, (iii) containing an average GC content of about 40 percent to about 60 percent, and (iv) complementary to a subsequence of a nucleic acid that does not repeat in the nucleic acid fragments; each of the oligonucleotide probe pairs in the set of oligonucleotide probe pairs comprises (i) a first oligonucleotide probe comprising a 5′ end about 2 to about 15 consecutive nucleotides from the 5′ end of the fragment, and (ii) a second oligonucleotide probe comprising a 3′ end about 2 to about 15 consecutive nucleotides from the 3′ end of the nucleic acid fragment to which the first oligonucleotide probe of the probe pair is capable of hybridizing; and each oligonucleotide probe in the set of single oligonucleotide probes comprises a 5′ or 3′ end about 2 to about 15 consecutive nucleotides from the 5′ end of a nucleic acid fragment.

Also described herein are methods for nucleic acid enrichment, comprising: subjecting target nucleic acid from a nucleic acid sample to nucleic acid cleavage conditions in which nucleic acid fragments are generated; subjecting the target nucleic acid fragments to linking conditions in which proximity ligated nucleic acid molecules are generated; contacting the proximity ligated nucleic acid molecules (or amplified copies thereof) with a composition comprising a plurality of oligonucleotide probes under hybridization conditions in which hybridization complexes comprising proximity ligated nucleic acid hybridized to oligonucleotide probes are generated; isolating the complexes; analyzing nucleic acid in the complexes, and; wherein plurality of oligonucleotide probes capable of hybridizing to target nucleic acid fragments of cancer gene exons, introns, promoters, and external regions surrounding cancer genes resulting from nucleic acid fragmentation of a nucleic acid sample comprise a plurality of external region directed oligonucleotide probes capable of hybridizing to target nucleic acid fragments from external regions comprises (i) a set of probe pairs each capable of hybridizing to a target nucleic acid fragment of about 260 consecutive nucleotides or longer, and (ii) a set of single oligonucleotide probes each capable of hybridizing to a nucleic acid fragment of about 130 consecutive nucleotides to about 260 consecutive nucleotides in length; the external region directed oligonucleotide probes in the set of oligonucleotide probe pairs and the set of single oligonucleotide probes comprise a polynucleotide (i) about 110 to about 130 consecutive nucleotides in length, (ii) substantially complementary to a subsequence of a nucleic acid fragment, (iii) containing an average GC content of about 40 percent to about 60 percent, and (iv) complementary to a subsequence of a nucleic acid that does not repeat in the nucleic acid fragments; each of the oligonucleotide probe pairs in the set of oligonucleotide probe pairs comprises (i) a first oligonucleotide probe comprising a 5′ end about 2 to about 15 consecutive nucleotides from the 5′ end of the fragment, and (ii) a second oligonucleotide probe comprising a 3′ end about 2 to about 15 consecutive nucleotides from the 3′ end of the nucleic acid fragment to which the first oligonucleotide probe of the probe pair is capable of hybridizing; and each oligonucleotide probe in the set of single oligonucleotide probes comprises a 5′ or 3′ end about 2 to about 15 consecutive nucleotides from the 5′ end of a nucleic acid fragment.

In any of the embodiments described herein, the cancer gene may be one or more gene listed in Appendix 1 or Appendix 2.

In any of the embodiments described herein, the oligonucleotide probes are capable of hybridizing to target nucleic acid fragments of external regions surrounding cancer genes.

In any of the embodiments described herein, the oligonucleotide probes comprise a capture agent and the solid phase comprises a capture agent counterpart suitable for binding to a capture agent of the oligonucleotide probes.

In any of the embodiments described herein, the capture agent and the capture agent counterpart independently are selected from biotin, avidin and streptavidin.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIGS. 1A and 1B show schematics of a proximity fusion.

FIGS. 2A and 2B show a schematic of the hypothetical detection of a proximity fusion using a method that preserves spatial proximity relationships (e.g. proximity ligation assays such as “Capture-HiC”, or others known in the art).

FIGS. 3A-3B show plots of how target gene size impacts proximity fusion signal, more so than distance of the target gene from breakpoint.

FIGS. 4A-4G show plots of showing how padding affects the proximity fusion signal.

FIG. 5 shows a schematic of an example of one embodiment of a probe design strategy for small genes in comparison to longer genes.

FIG. 6 shows a schematic of an example of one embodiment of a probe design strategy.

FIG. 7 shows a schematic of an example of one embodiment of a probe design strategy for a Group 1 gene focusing on promoter(s) and exon(s).

FIG. 8 shows a schematic of one example of an embodiment of a probe design strategy for a Group 1 gene focusing on intron(s).

FIG. 9 shows a schematic of an example of one embodiment of probe placement around a Group 1 gene, focusing on probes targeting the upstream and downstream regions (also known as the extension regions) that are associated with increasing the length of targeting to 100 kb for a small gene.

FIG. 10 shows a schematic of an example of one embodiment of probe placement around a Group 1 gene, focusing on the additional probes in the up/downstream padding regions beyond the gene (or in this case beyond the extension regions because MYC is a small gene).

FIG. 11 shows one example of an embodiment of probe placement around a Group 2 gene.

FIG. 12 shows a table of different probe design sets in accordance with various embodiments of the invention.

FIG. 13 shows graphical results of experiments using 4 different probe design sets in accordance with embodiments of the invention.

FIG. 14 shows graphical results of experiments using 4 different probe design sets using an FFPE gynecological sarcoma tumor specimen in accordance with embodiments of the invention.

FIG. 15 shows graphical results of experiments using 4 different probe design sets using an FFPE gynecological sarcoma tumor specimen in accordance with embodiments of the invention.

FIG. 16 shows a Manhattan plot using 0 kb of additional padding in accordance with an embodiment of the invention.

FIG. 17 shows a Manhattan plot using 100 kb of additional padding in accordance with an embodiment of the invention.

FIG. 18 shows a Manhattan plot using 250 kb of additional padding in accordance with an embodiment of the invention.

FIG. 19 shows a Manhattan plot using 500 kb of additional padding in accordance with an embodiment of the invention.

FIG. 20 shows a Manhattan plot using 0 kb of additional padding in accordance with an embodiment of the invention.

FIG. 21 shows a Manhattan plot using 100 kb of additional padding in accordance with an embodiment of the invention.

FIG. 22 shows a Manhattan plot using 250 kb of additional padding in accordance with an embodiment of the invention.

FIG. 23 shows a Manhattan plot using 500 kb of additional padding in accordance with an embodiment of the invention.

FIG. 24 shows a Manhattan plot using 0 kb of additional padding in accordance with an embodiment of the invention.

FIG. 25 shows a Manhattan plot using 100 kb of additional padding in accordance with an embodiment of the invention.

FIG. 26 shows a Manhattan plot using 250 kb of additional padding in accordance with an embodiment of the invention.

FIG. 27 shows a Manhattan plot using 500 kb of additional padding in accordance with an embodiment of the invention.

### APPENDICES

Appendix 1 presents a list of cancer genes containing polynucleotide regions to which oligonucleotide probes can hybridize, and/or to which oligonucleotide probes can be designed to hybridize, in certain implementations. Appendix 1 shows the name of the cancer gene, the chromosome on which the cancer gene is located, the start and end positions of the cancer gene, according to coordinate positions from the Genome Reference Consortium Human Build 38 (GRCH38), and on which Watson (+) or Crick (−) strand the gene is oriented in the sense direction.

Appendix 2 presents a list of cancer genes containing polynucleotide regions to which oligonucleotide probes can hybridize, and/or to which oligonucleotide probes can be designed to hybridize, in certain implementations. Appendix 2 shows the name of the cancer gene, the chromosome on which the cancer gene is located, the start and end positions of the cancer gene (see columns “gene start” and “gene end”), according to coordinate positions from the Genome Reference Consortium Human Build 38 (GRCH38), and on which Watson (+) or Crick (−) strand the gene is oriented in the sense direction. Appendix 2 also shows the start and end positions of the target region for the cancer gene (see columns headed with “target start” and “target end”. If the cancer gene >100 kb, the target region for the cancer gene has the same coordinates as the cancer gene itself. If the cancer gene <100 kb, the target region for the cancer gene has been extended by adding extension regions to increase the effective length of the cancer gene to approximately 100 kb. Appendix 2 also shows the target length, which is the genomic length of the target region. For cancer genes >100 kb, the target length is equal to the cancer gene length. For cancer genes <100 kb, the target length is equal to the gene length plus the extension regions, which should sum to approximately 100 kb. Appendix 2 also shows an Applicant determined Group 1 or Group 2 designation for each cancer gene listed.

### DETAILED DESCRIPTION

Oligonucleotide probe compositions described herein can be utilized to (i) detect structural variant (SV) break points in exons in the genes in the panel; (ii) detect SV break points in introns in the genes in the panel; (iii) detect SV break points upstream or downstream of the genes in the panel (e.g., neighborhood SVs, also known as proximal or proximity fusions); (iv) identifying novel looping interactions with each of the genes, referred to as “neoloops,” associated with breakpoints in or near the genes in the panel; (v) detecting SV's in FFPE tissues; or (vi) a combination of two or more of any of (i), (ii), (iii), (iv) and (v). Thus, oligonucleotide probe compositions described herein provide advantages of (i) detecting SV's in exons as well as in introns and external regions (ii) detecting SV's in FFPE tissues; (iii) detecting SV's in solid tumor samples; and (iv) detecting SV's in undiagnosed tumor samples.

Oligonucleotide probe compositions (e.g., a panel of oligonucleotide probes that hybridize to or target one or more of the cancer genes presented in Appendix 1 and Appendix 2) may be generated using one or more design methodologies as described herein. In some embodiments, a panel of oligonucleotide probes are generated using a sparse design. In some embodiments, a sparse design for generating a panel of oligonucleotide probes involves constraining the panel to consist of oligonucleotide probes having a % GC content of 40% to 60%, constraining the panel such that discrete oligonucleotide probes do not comprise overlapping cut sites with other probes within the panel, and/or reducing the number of predicted low-performance (e.g., high off-target) oligonucleotide probes. In some embodiments, a sparse design for generating a panel of oligonucleotide probes involves constraining the panel to consist of oligonucleotide probes having a % GC content of 40% to 60%, constraining the panel such that discrete oligonucleotide probes do not comprise a restriction cut site or hybridize to a region of the target that comprises a restriction cut site, and/or reducing the number of predicted low-performance (e.g., high off-target) oligonucleotide probes. In some embodiments, a panel of oligonucleotide probes that target intronic regions (e.g., of a cancer gene) are generated using a sparse design. In some embodiments, a panel of oligonucleotide probes that target genomic areas outside a target gene are generated using a sparse design. Herein, genomic areas outside a target gene (e.g. a cancer gene of interest) will be referred to as external regions. External region means any genomic region that is not part of a (target) gene or its promoter. Herein, there are at least two types of external regions, namely an extension region and a padding region. An extension region is a genomic region directly outside a target gene and its promoter (e.g. for a cancer gene of interest) that serves to extend the effective length of certain target genes. For example, relatively small target genes may have extension region(s) to extend their effective length from relatively small to larger regions. A padding region is a genomic region outside of a target gene and its promoter (e.g. for a cancer gene of interest) for genes that do not have extension regions, or, a genomic region outside of the extension region(s) for target genes that have extension regions. Extension regions and padding regions are non-overlapping types of external regions. A gene is defined herein as all the sequence between the upstream most transcription start site and the downstream most transcription stop site. A promoter is defined herein as the sequence between 1500 bp upstream from the upstream most transcription start site and 500 bp downstream of the upstream most transcription start site. In some embodiments, external regions can encompass 10 kb, 25 kb, 50 kb, 100 kb, 125 kb, 150 kB, 175 kb, 200 kb, 250 kb, 300 kb, 400 kb, 500 kb, 750 kb, 1000 kb, 1500 kb, 2000 kb, 5000 kb, or 10,000 kb upstream and/or downstream of the target gene. In some cases an external region may include a portion of or all of another gene nearby to the target gene. Such sequences are included in the definition of external region, as those sequences are still external to whichever target gene is being investigated.

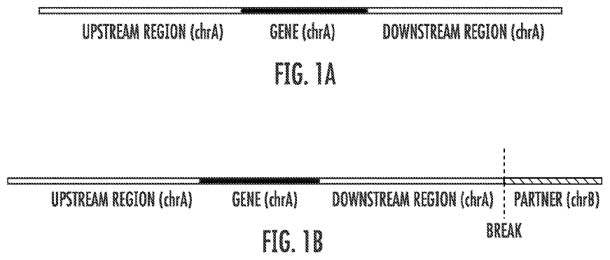

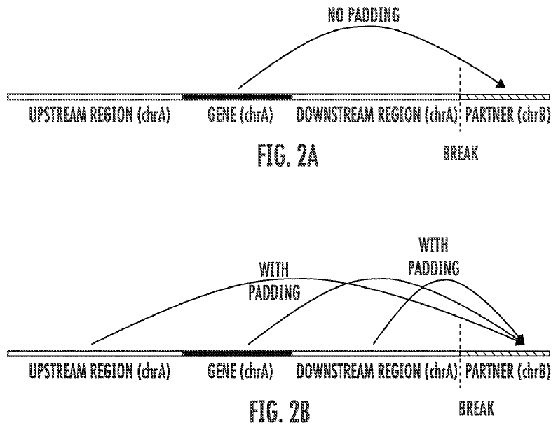

Sparse design (e.g., sparse design for external regions) can, in some embodiments, add more complexity to the subsequent Hic capture data than traditional approaches. For example, sparse design enables the capture of long-range genomic information when using a panel of oligonucleotide probes that target a limited region of the genome, making it easier for bioinformatics pipelines to identify SVs and to make the collected data more similar to a genome-wide HiC experiment. Thus, in some embodiments, sparse design provides the low cost of sequencing benefits of capture, while retaining the useful information derived from genome-wide sequencing approaches. Traditional approaches for HiC capture have relied on panels of oligonucleotide probes that are designed to target exons. Applicants have recently discovered however that some breakpoints for structural variants of target genes can occur in the external regions. By designing oligonucleotide probe compositions to include some sequences that align to these external regions, the signal of the proximity ligations assay can be increased. FIGS. 1 and 2 illustrate the mechanism for this outcome. FIG. 1 shows schematics of a hypothetical gene A on hypothetical chromosome A. FIG. 1A shows gene A along with genomic regions upstream and downstream of gene A on chromosome A. FIG. 1B shows a hypothetical structural variant made from gene A on chromosome A fused to a partner region on chromosome B. The vertical dotted line represents the breakpoint. FIG. 2 shows a schematic of the hypothetical detection of a proximity fusion using a method that preserves spatial proximity relationships (e.g. proximity ligation assays such as “Capture-HiC”, or others known in the art). FIG. 2A shows a potential contact point between gene A and the partner region when the proximity ligation assay uses probe sets known in the art (generally probes that align to exons of target gene, but may also include introns). FIG. 2B shows potential contact points between gene A and gene A external regions with the partner region when the proximity ligation assay uses probe sets that include probes designed to hybridize to external regions. The resulting increase in contact points allows for a more robust signal generated by the proximity ligation assay and can thus allow for easier detection of breakpoints without a concordant increase in sequencing costs.

### Oligonucleotide Probe Compositions

Provided in certain aspects is a composition that includes: a plurality of oligonucleotide probes capable of hybridizing to target nucleic acid fragments of cancer gene exons, introns, untranslated regions and external regions surrounding cancer genes resulting from a process comprising nucleic acid restriction enzyme cleavage or other fragmentation of a nucleic acid sample, where: a plurality of oligonucleotide probes capable of hybridizing to target nucleic acid fragments of external regions comprises (i) a set of probe pairs each capable of hybridizing to a target nucleic acid fragment of about 260 consecutive nucleotides or longer, and (ii) a set of single oligonucleotide probes each capable of hybridizing to a nucleic acid fragment of about 130 consecutive nucleotides to about 260 consecutive nucleotides in length; the probes in the set of oligonucleotide probe pairs and the set of single oligonucleotide probes comprise a polynucleotide (i) about 110 to about 130 consecutive nucleotides in length, (ii) substantially complementary to a subsequence of a nucleic acid fragment, (iii) containing an average GC content of about 40 percent to about 60 percent, and (iv) complementary to a subsequence of a nucleic acid that does not repeat in the nucleic acid fragments; each of the probe pairs in the set of probe pairs comprises (i) a first oligonucleotide probe comprising a 5′ end about 2 to about 15 consecutive nucleotides from the 5′ end of the fragment, and (ii) a second oligonucleotide probe comprising a 3′ end about 2 to about 15 consecutive nucleotides from the 3′ end of the nucleic acid fragment to which the first oligonucleotide probe of the probe pair is capable of hybridizing; and each probe in the set of single oligonucleotide probes comprises a 5′ or 3′ end about 2 to about 15 consecutive nucleotides from the 5′ end of a nucleic acid fragment.

The term “target nucleic acid fragments of external regions” generally refers to target nucleic acid fragments generated by fragmentation of gene external regions. The term “probe pair” generally refers to two probes designed to specifically hybridize to a fragment, where one probe is designed to hybridize near the 5′ end of the fragment and the other probe is designed to hybridize near the 3′ end of the fragment. The term “external region directed” oligonucleotide probes generally refers to oligonucleotide probes capable of hybridizing to, and/or designed to hybridize to, target nucleic acid fragments of external regions. The term “single probe” generally refers to a probe designed to specifically hybridize near the 5′ end of a fragment, where the fragment typically hybridizes to a single probe, and not a probe pair, in the plurality of oligonucleotide probes.

In certain implementations, the oligonucleotide probes in the set of oligonucleotide probe pairs and the set of single oligonucleotide probes, and/or oligonucleotide probes in the composition, are capable of hybridizing to nucleic acid fragments from 100 or more cancer genes. In certain instances, the oligonucleotide probes in the set of oligonucleotide probe pairs and the set of single oligonucleotide probes, and/or oligonucleotide probes in the composition, are capable of hybridizing to nucleic acid fragments from about 500 or more cancer genes, about 800 or more cancer genes, about 1000 or more cancer genes, about 1200 or more cancer genes, or about 1400 or more cancer genes. In certain implementations, the oligonucleotide probes in the set of oligonucleotide probe pairs and the set of single oligonucleotide probes, and/or oligonucleotide probes in the composition, are capable of hybridizing to nucleic acid fragments from 100 or more cancer genes listed in Appendix 1. In certain instances, the oligonucleotide probes in the set of oligonucleotide probe pairs and the set of single oligonucleotide probes, and/or oligonucleotide probes in the composition, are capable of hybridizing to nucleic acid fragments from about 500 or more cancer genes listed in Appendix 1, about 800 or more cancer genes listed in Appendix 1, about 1000 or more cancer genes listed in Appendix 1, about 1200 or more cancer genes listed in Appendix 1, or about 1400 or more cancer genes listed in Appendix 1.

In certain implementations, the oligonucleotide probes in the set of oligonucleotide probe pairs and the set of single oligonucleotide probes, and/or oligonucleotide probes in the composition, are capable of hybridizing to nucleic acid fragments from 25 or more cancer genes. In certain instances, the oligonucleotide probes in the set of oligonucleotide probe pairs and the set of single oligonucleotide probes, and/or oligonucleotide probes in the composition, are capable of hybridizing to nucleic acid fragments from about 20 or more cancer genes, about 50 or more cancer genes, about 75 or more cancer genes, about 100 or more cancer genes, or about 130 or more cancer genes. In certain implementations, the oligonucleotide probes in the set of oligonucleotide probe pairs and the set of single oligonucleotide probes, and/or oligonucleotide probes in the composition, are capable of hybridizing to nucleic acid fragments from 25 or more cancer genes listed in Appendix 2.

In certain instances, the oligonucleotide probes in the set of oligonucleotide probe pairs and the set of single oligonucleotide probes, and/or oligonucleotide probes in the composition, are capable of hybridizing to nucleic acid fragments from about 20 or more cancer genes listed in Appendix 2, about 50 or more cancer genes listed in Appendix 2, about 75 or more cancer genes listed in Appendix 2, about 100 or more cancer genes listed in Appendix 2, or about 130 or more cancer genes listed in Appendix 2.

In certain instances, the polynucleotide of each of the oligonucleotide probes in the set of oligonucleotide probe pairs and the set of single oligonucleotide probes, and/or in the composition, is about 120 consecutive nucleotides. In certain implementations, the polynucleotide of each of the oligonucleotide probes in the set of oligonucleotide probe pairs and the set of single oligonucleotide probes, and/or in the composition, is complementary to a subsequence of a nucleic acid fragment.

In certain instances, the polynucleotide of each of the oligonucleotide probes in the set of oligonucleotide probe pairs and the set of single oligonucleotide probes, and/or the polynucleotide of each of the oligonucleotide probes in the composition, is 100% complementary to a corresponding portion of the minus strand of Genome Reference Consortium Human Build 38 (GRCH38). In certain instances, the polynucleotide of each of the oligonucleotide probes in the set of oligonucleotide probe pairs and the set of single oligonucleotide probes, and/or the polynucleotide of each of the oligonucleotide probes in the composition, is 100% complementary to a corresponding portion of the plus strand of Genome Reference Consortium Human Build 38 (GRCH38). In certain instances, the composition comprises a mixture of (i) polynucleotides of oligonucleotide probes that are 100% complementary to a corresponding portion of the minus strand of Genome Reference Consortium Human Build 38 (GRCH38) and (ii) polynucleotides of oligonucleotide probes that are 100% complementary to a corresponding portion of the minus strand of Genome Reference Consortium Human Build 38 (GRCH38). In certain implementations, each of the oligonucleotide probes in the set of oligonucleotide probe pairs and the set of single oligonucleotide probes, and/or each of the oligonucleotide probes in the composition, is capable of hybridizing to a fragment under hybridization conditions of moderate stringency and/or high stringency. Hybridization and hybridization conditions of different stringency are described herein. In certain instances, the polynucleotides of the external region directed oligonucleotide probes in the set of oligonucleotide probe pairs and the set of single oligonucleotide probes, and/or of each of the oligonucleotide probes in the composition, are capable of hybridizing to target nucleic acid fragments having one or more of the following features: (i) an average fragment size of about 180 consecutive nucleotides to about 200 consecutive nucleotides (e.g., about 190 or 191 consecutive nucleotides); (ii) a GC content of about 1% (e.g., about 1.53%) to about 97% (e.g., about 96.6%); and (iii) an average GC content of about 40% to about 45% (e.g., an average GC content of about 43%). In certain instances, the polynucleotides of the external region directed oligonucleotide probes in the set of oligonucleotide probe pairs and/or the set of single oligonucleotide probes do not comprise polynucleotides having a GC content of less than about 40% GC content or higher than about 60% GC content.

In certain implementations, target nucleic acid fragments result from nucleic acid restriction enzyme cleavage. Restriction enzyme cleavage sometimes is performed by one or more restriction endonucleases, such as by restriction enzymes cutting at \hat{ }GATC and G\hat{ }ANTC, where “\hat{ }” represents the cut site on the positive DNA strand (i.e., Watson strand), for example.

In certain instances, target nucleic acid fragments result from a process that includes digestion of sample nucleic acid by two, three, four, five or six restriction enzymes. In certain implementations, target nucleic acid fragments result from a process that includes digestion of nucleic acid by one or more restriction enzymes chosen from one or more of HpyCH4IV, HinfI, HinP1I and MseI. In certain instances, target nucleic acid fragments result from a process that includes digestion of nucleic acid by one or more restrictions enzymes chosen from one or more of HindIII, DpnII, MboI and NlaIII. In certain implementations, target nucleic acid fragments result from a process that includes a combination of (i) digestion of nucleic acid by one or more restriction enzymes and (ii) physical fragmentation of nucleic acid (e.g., sonication, shearing).

Polynucleotides of oligonucleotide probes can be designed by generating a set of target nucleic acid fragments by an in silico process that includes in silico cleavage of nucleic acid, and analyzing polynucleotide targets within the target nucleic acid fragments generated in silico for the design of complementary polynucleotides of oligonucleotide probes capable of hybridizing to the polynucleotide targets. In some embodiments, the oligonucleotide probes are designed such that the probes do not hybridize to a target sequence that contains a restriction cut site. Any other method of fragmentation nucleic acids known in the art can be used.

A polynucleotide of each of the oligonucleotide probes in a composition often is synthetic. A polynucleotide of an oligonucleotide probe can include any backbone and base combination suitable for the oligonucleotide probe to specifically hybridize to a target nucleic acid (e.g., a target nucleic acid fragment). In certain instances, a polynucleotide of each of the oligonucleotide probes includes RNA or DNA, a modified backbone of RNA or DNA, one or more modified DNA or RNA bases, or a combination of two or more of the foregoing.

In certain implementations, oligonucleotide probes in a composition include a capture agent. In such implementations, a polynucleotide of an oligonucleotide probe often is modified by the capture agent and is not naturally occurring. Any suitable capture agent that can specifically bind to a capture agent counterpart (e.g., a capture agent counterpart linked to a solid phase) can be associated with an oligonucleotide probe. A capture agent and capture agent counterpart sometimes are members of a binding pair. Non-limiting examples of binding pairs include an antigenic epitope and an antibody or immunologically reactive fragment thereof; an antibody and a hapten; a digoxigenin moiety and an anti-digoxigenin antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; biotin and avidin; biotin and streptavidin; the like or combinations thereof. A capture agent and a capture agent counterpart sometimes include, independently, biotin, avidin or streptavidin. A capture agent sometimes is covalently attached to an oligonucleotide probe, sometimes is linked to a 5′ end and/or 3′ end of an oligonucleotide probe, and/or sometime is linked to one or more nucleotide bases of an oligonucleotide probe (e.g., linked to one or more bases chosen from adenine, cytosine, guanine, thymine or uracil of an oligonucleotide probe). Methods for associating a capture agent with an oligonucleotide probe are known in the art.

In certain implementations, oligonucleotide probes capable of hybridizing to target nucleic acid fragments of external regions and/or introns are not capable of hybridizing to tiled subsequences of a target nucleic acid fragment. In certain instances, oligonucleotide probes capable of hybridizing to target nucleic acid fragments of external regions and/or introns are not capable of hybridizing to contiguous regions of the nucleic acid fragment. The term “contiguous regions of a nucleic acid fragment” is synonymous with the term “tiled” and generally refers to target polynucleotide regions of a target nucleic acid fragment, to which oligonucleotide probes are capable of hybridizing, that are disposed end to end in the target nucleic acid fragment with no gap, or a short gap of 1 nucleotide to about 5 consecutive nucleotides, between the ends of adjacent target polynucleotide regions in the fragment. There sometimes are no gaps between the ends of adjacent (tiled) oligonucleotide probes hybridized to contiguous regions of a nucleic acid fragment. In certain instances, the contiguous (tiled) target polynucleotide regions of a target nucleic acid fragment, to which oligonucleotide probes are capable of hybridizing, are disposed end to end in the target nucleic acid fragment with a gap between the ends of adjacent target polynucleotide regions in the fragment, where the gap can be 1 nucleotide, 2 consecutive nucleotides, 3 consecutive nucleotides, 4 consecutive nucleotides, or 5 consecutive nucleotides or any combination thereof.

In certain instances, the plurality of oligonucleotide probes capable of hybridizing to target nucleic acid fragments of external regions and/or introns does not contain a probe capable of hybridizing to an end of a first target nucleic acid fragment and to an end of a second target nucleic acid fragment (e.g., external region directed and/or intronic directed probes are not designed to a region overlapping a restriction enzyme cleavage site, and therefore are not designed to hybridize to, under hybridization conditions, two target nucleic acid fragments containing adjacent polynucleotides in non-cleaved target nucleic acid). In certain instances, the plurality of oligonucleotide probes capable of hybridizing to target nucleic acid fragments of external regions and/or introns does not contain an oligonucleotide probe capable of hybridizing to, or does not contain an oligonucleotide probe designed to hybridize to, a target nucleic acid fragment having a length of less than 130 consecutive nucleotides (e.g., external region directed probes are not designed to hybridize to, under hybridization conditions, target nucleic acid fragment of a length of less than 130 consecutive nucleotides). In certain instances, the plurality of oligonucleotide probes in the composition does not contain an oligonucleotide probe capable of hybridizing to, or does not contain an oligonucleotide probe designed to hybridize to, a target nucleic acid fragment having a length of less than 130 consecutive nucleotides (e.g., oligonucleotide probes in the composition are not designed to hybridize to, under hybridization conditions, target nucleic acid fragment of a length of less than 130 consecutive nucleotides). In certain implementations, the first oligonucleotide probe and the second oligonucleotide probe in each of the oligonucleotide probe pairs do not hybridize to regions of a target nucleic acid fragment that overlap. In certain implementations, oligonucleotide probes in the composition do not hybridize to regions of a target nucleic acid fragment that overlap.

When the external regions and/or introns are fragmented, some resulting nucleic acid fragments will be of a length sufficient to hybridize at least a pair of oligonucleotide probes, such length being at least about 220 consecutive nucleotides, at least about 240 consecutive nucleotides, at least about 260 consecutive nucleotides, at least about 280 consecutive nucleotides or at least about 300 consecutive nucleotides. When external regions and/or introns are fragmented, some resulting nucleic acid fragments will be of a length sufficient to hybridize only a single oligonucleotide probe, such length being about 120 consecutive nucleotides to about 140 consecutive nucleotides in length (e.g., about 130 consecutive nucleotides in length), or no more than about 220 consecutive nucleotides to about 280 consecutive oligonucleotides in length (e.g., no more than about 230 m 240, 250, 260, 270 or 280 consecutive nucleotides in length). When external regions and/or introns are fragmented, the resulting nucleic acid fragments typically are a plurality of sizes, often including (i) nucleic acid fragments of sizes sufficient to hybridize at least a pair of oligonucleotide probes and (ii) nucleic acid fragments of a length sufficient to hybridize only a single oligonucleotide probe, and sometimes including (iii) nucleic acid fragments of a length insufficient to hybridize to any oligonucleotide probe. Accordingly, in certain embodiments, the composition may include a plurality of oligonucleotide probes capable of hybridizing to external regions and/or introns comprising (i) a set of probe pairs each capable of hybridizing to a target nucleic acid fragment of about 260 consecutive nucleotides or longer, and (ii) a set of single oligonucleotide probes each capable of hybridizing to a nucleic acid fragment of about 130 consecutive nucleotides to about 260 consecutive nucleotides in length. In certain implementations, each oligonucleotide probe of a plurality of oligonucleotide probes capable of hybridizing to target nucleic acid fragments of exons and promoters is capable of hybridizing to a region spanning about 100 to about 500 consecutive nucleotides (e.g., about 350 consecutive nucleotides) from the 5′ end of each target nucleic acid fragment or to a region spanning about 100 to about 500 consecutive nucleotides (e.g., about 350 consecutive nucleotides) from the 3′ end of each target nucleic acid fragment. In certain instances, the oligonucleotide probes of the plurality of oligonucleotide probes capable of hybridizing to target nucleic acid fragments of exons and promoters capable of hybridizing to a target nucleic acid fragment are capable of hybridizing to contiguous, non-overlapping regions of the nucleic acid fragment. In certain instances, the oligonucleotide probes of the plurality of oligonucleotide probes capable of hybridizing to target nucleic acid fragments of exons and promoters are not restricted to a GC content range (e.g., not restricted to a GC percentage range) or to a GC content threshold (e.g., not restricted to a GC percentage threshold). The term “target nucleic acid fragments of exons and promoters” generally refers to target nucleic acid fragments generated by fragmentation of gene exon regions and to target nucleic acid fragments generated by fragmentation of gene promoter regions.

The term “exon-directed” and “promoter-directed” oligonucleotide probes generally refers to oligonucleotide probes capable of hybridizing to, and/or designed to hybridize to, target nucleic acid fragments of exons and promoters, respectively.

The term “abundance” of each oligonucleotide probe in a composition generally refers to the amount of the oligonucleotide probe in the composition, which sometimes is a molar concentration for example. The abundance of one or more oligonucleotide probes in a composition sometimes is a relative abundance, relative to the abundance of one or more other oligonucleotide probes, and sometimes is expressed as a molar ratio for example. The abundance of at least one oligonucleotide probe in a composition sometimes is the same or about the same as the abundance of another oligonucleotide probe in the composition. The abundance of each oligonucleotide probe in a composition sometimes is the same or about the same as the abundance of each other oligonucleotide probe in the composition. In certain implementations, the abundance of at least one external region directed oligonucleotide probe in a composition sometimes is the same or about the same as the abundance of another external region directed oligonucleotide probe in the composition. In certain instances, the abundance of each external region directed oligonucleotide probe in the composition is the same or about the same as each other external region directed oligonucleotide probe.

In certain implementations, the abundance of one or more oligonucleotide probes in a composition is different than the abundance of one or more other oligonucleotide probes in the composition. For example, the abundance of a first oligonucleotide probe in a composition can be lower or higher than the abundance of a second oligonucleotide probe in the composition. The abundance of one or more exon-directed oligonucleotide probes sometimes is different than the abundance of one or more other exon-directed oligonucleotide probes. The abundance of one or more promoter-directed oligonucleotide probes sometimes is different than the abundance of one or more other promoter-directed oligonucleotide probes. The abundance of an oligonucleotide probe can be different than the abundance of another oligonucleotide probe in a composition to normalize the amount of target nucleic acid hybridized to each of the probes. For example, where a first target nucleic acid is present in a sample at an abundance higher than the abundance of a second target nucleic acid in the sample, the abundance of a first oligonucleotide probe that specifically hybridizes to the first target nucleic acid fragment can be lower than the abundance of a second oligonucleotide probe that specifically hybridizes to the second target nucleic acid fragment, to normalize the amount of target nucleic acid hybridized to each of the oligonucleotide probes. The abundance of at least a subset of oligonucleotide probes in a composition can be selected, for the purpose of normalizing the amount of target nucleic acid hybridized for example, by methods known in the art (e.g., by utilizing AGILENT SUREDESIGN™ software or other software products).

In certain implementations, the abundance of one or more designed oligonucleotide probes in the composition, or the relative abundance of one or more designed oligonucleotide probes relative to one or more other oligonucleotide probes, can be selected. Oligonucleotide probe abundance is described herein.

In an embodiment, capture probes can be clustered together with spaces between the clusters. The clusters can have uniform or non-uniform distance between them and the clusters can be within the gene body or can be in adjacent regions around the gene or in both locations.

In an embodiment, capture probes cover all restriction fragments in each gene in the panel.

In an embodiment, capture probes cover select restriction fragments in each gene in the panel.

In an embodiment, capture probes cover select restriction fragments in each gene in the panel resulting in uniform coverage through the gene.

In an embodiment, capture probes cover select restriction fragments in each gene in the panel resulting in non-uniform coverage through the gene.

In an embodiment, capture probes cover select restriction fragments in each gene in the panel based on estimated performance metrics of the probes, resulting in non-uniform coverage through the gene.

In an embodiment, capture probes cover select restriction fragments in each gene in the panel resulting in high density coverage over promoters and high density coverage through the rest of gene.

In an embodiment, capture probes cover select restriction fragments in each gene in the panel resulting in high density coverage over promoters and low density coverage through the rest of gene.

In an embodiment, capture probes cover select restriction fragments in each gene in the panel resulting in low density coverage over promoters and high density coverage through the rest of gene.

In an embodiment, capture probes cover select restriction fragments in each gene in the panel resulting in low density coverage over promoters and low density coverage through the rest of gene.

In an embodiment, capture probes cover select restriction fragments in each gene in the panel resulting in high density coverage over exons and high density coverage through the rest of gene.

In an embodiment, capture probes cover select restriction fragments in each gene in the panel resulting in high density coverage over exons and low density coverage through the rest of gene.

In an embodiment, capture probes cover select restriction fragments in each gene in the panel resulting in low density coverage over exons and high density coverage through the rest of gene.

In an embodiment, capture probes cover select restriction fragments in each gene in the panel resulting in low density coverage over exons and low density coverage through the rest of gene.

In an embodiment, capture probes cover select restriction fragments in each gene in the panel resulting in high density coverage over introns and high density coverage through the rest of gene.

In an embodiment, capture probes cover select restriction fragments in each gene in the panel resulting in high density coverage over introns and low density coverage through the rest of gene.

In an embodiment, capture probes cover select restriction fragments in each gene in the panel resulting in low density coverage over introns and high density coverage through the rest of gene.

In an embodiment, capture probes cover select restriction fragments in each gene in the panel resulting in low density coverage over introns and low density coverage through the rest of gene.

In an embodiment, capture probes cover select restriction fragments in each gene in the panel resulting in high density coverage over promoters and introns and high density coverage through the rest of gene.

In an embodiment, capture probes cover select restriction fragments in each gene in the panel resulting in high density coverage over promoters and introns and low density coverage through the rest of gene.

In an embodiment, capture probes cover select restriction fragments in each gene in the panel resulting in low density coverage over promoters and introns and high density coverage through the rest of gene.

In an embodiment, capture probes cover select restriction fragments in each gene in the panel resulting in low density coverage over promoters and introns and low density coverage through the rest of gene.

In an embodiment, capture probes cover select restriction fragments in each gene in the panel resulting in high density coverage over promoters and exons and high density coverage through the rest of gene.

In an embodiment, capture probes cover select restriction fragments in each gene in the panel resulting in high density coverage over promoters and exons and low density coverage through the rest of gene.

In an embodiment, capture probes cover select restriction fragments in each gene in the panel resulting in low density coverage over promoters and exons and high density coverage through the rest of gene.

In an embodiment, capture probes cover select restriction fragments in each gene in the panel resulting in low density coverage over promoters and exons and low density coverage through the rest of gene.

In an embodiment, capture probes cover select restriction fragments in each gene in the panel resulting in high density coverage over promoters, introns, and exons and high density coverage through the rest of gene.

In an embodiment, capture probes cover select restriction fragments in each gene in the panel resulting in high density coverage over promoters, introns, and exons and low density coverage through the rest of gene.

In an embodiment, capture probes cover select restriction fragments in each gene in the panel resulting in low density coverage over promoters, introns, and exons and high density coverage through the rest of gene.

In an embodiment, capture probes cover select restriction fragments in each gene in the panel resulting in low density coverage over promoters, introns, and exons and low density coverage through the rest of gene.

In an embodiment, capture probes cover all restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2.

In an embodiment, capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2.

In an embodiment, capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in uniform coverage through the “padding” region.

In an embodiment, capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in non-uniform coverage through the “padding” region.

In an embodiment, capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 based on estimated performance metrics of the probes, resulting in non-uniform coverage through the “padding” region.

In an embodiment, capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in “padding” regions of any size including 10 kb, 50 kb, the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In one embodiment, oligonucleotide probes capable of hybridizing to target nucleic acid fragments of cancer gene exons, introns, untranslated regions and external regions surrounding cancer genes (sometimes referred to “capture probes” throughout) cover select restriction fragments in one or more cancer gene resulting in high density coverage over promoters and high density coverage through the rest of gene. In certain embodiments, capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In other embodiments, capture probes cover select restriction fragments in each gene in the panel resulting in high density coverage over promoters and low density coverage through the rest of gene. In other embodiments, capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and low density coverage through the rest of gene. In an embodiment, capture probes cover select Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over exons and high density coverage through the rest of gene. In an embodiment, capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over exons and high density coverage through the rest of the gene and the capture probes cover select restriction cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over introns and low density coverage through the rest of the gene and the capture probes cover select restriction cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over introns and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters and introns and high density coverage Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters and introns and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and introns and low density coverage Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and exons and high density coverage Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters, introns, and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters, introns, and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters, introns, and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters, introns, and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters and high density coverage through the cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and low density coverage through the cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over exons and high density coverage through the rest of the gene and the capture probes cover select restriction cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over introns and low density coverage through the rest of the gene and the capture probes cover select restriction cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over introns and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters and introns and high density coverage Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters and introns and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and introns and low density coverage Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and exons and high density coverage Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters, introns, and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters, introns, and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters, introns, and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters, introns, and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters and high density coverage through the cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and low density coverage through the cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over exons and high density coverage through the rest of the gene and the capture probes cover select restriction cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over introns and low density coverage through the rest of the gene and the capture probes cover select restriction cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over introns and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters and introns and high density coverage Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters and introns and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and introns and low density coverage Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and exons and high density coverage Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters, introns, and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters, introns, and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters, introns, and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in low density coverage over promoters, introns, and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and high density coverage through the rest of the “padding” region.

In an embodiment, capture probes cover select restriction fragments in each gene in the panel resulting in high density coverage over promoters and high density coverage through the cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in high density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and low density coverage through the cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including
“Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over exons and high density coverage through the rest of the gene and the capture probes cover select restriction fragments in “padding” regions of any size including 10 kb, 50 kb, the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including
“Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over introns and low density coverage through the rest of the gene and the capture probes cover select restriction fragments in “padding” regions of any size including 10 kb, 50 kb, the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over introns and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters and introns and high density coverage restriction fragments in “padding” regions of any size including adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters and introns and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and introns and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and introns and low density coverage restriction fragments in “padding” regions of any size including adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and exons and high density coverage restriction fragments in “padding” regions of any size including adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters, introns, and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in high density coverage over promoters, introns, and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters, introns, and exons and high density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select restriction fragments in each gene in the panel resulting in low density coverage over promoters, introns, and exons and low density coverage through the rest of the gene and the capture probes cover select restriction fragments in “padding” regions of any size including 10 kb, 50 kb, 100 kb, 250 kb, 500 kb, 1 Mb, 2 Mb 5 Mb, 10 Mb, 20 Mb adjacent to either the 5′ or 3′ end or to both the 5′ and 3′ ends of one or more cancer genes listed in Appendix 1 or Appendix 2 resulting in low density coverage over “DNA Regulatory Element” including “Enhancers”, Repressors”, “CTCF Sites”, or other “DNA Regulatory Elements” and low density coverage through the rest of the “padding” region.

In an embodiment, capture probes cover select “control” restriction fragments in regions outside each gene in the panel.

In an embodiment, capture probes cover select “control” restriction fragments in regions outside each gene in the panel which cover consecutive fragments.

In an embodiment, capture probes cover select “control” restriction fragments in regions outside each gene in the panel which cover fragments sporadically throughout the genome. In one embodiment, gRNA's (Guide-RNA's) are used in a complex with CRISPR-Cas9 digest restriction fragment in regions not in the gene panel.

In an embodiment, the target genes include all the genes in the Appendix 2.

In an embodiment, the capture probes provide 100% of coverage for all restriction cut sites for exons regardless of fragment size.

In an embodiment, the capture probes provide 15% coverage for introns regardless of cut site size.

In an embodiment, the capture probes provide 100% of coverage for all promoters (i.e. 1500 bp upstream to 500 bp downstream) In an embodiment, the capture probes provide 15% of coverage, regardless of fragment size, for padding regions upstream and downstream of the target genes.

In an embodiment, target genes less than 100 kb will have padding regions added to bring them up to a 100 kb total target size. In an embodiment, Group 1 genes (as designated in Appendix 2) have additional padding (0, 100 kb, 250 kb, and/or 500 kb). In an embodiment, Group 2 genes (as designated in Appendix 2) have no additional padding).

In an embodiment, a composition of capture probes to multiple target genes does not exceed 6 Mb. In an embodiment, the padding regions of Group 1 genes (as designated in Appendix 2) are adjusted to stay within a 6 Mb limit of a composition of capture probes to multiple target genes.

In an embodiment, sequencing reads do not exceed 5M PE reads.

### Methods for Designing and Manufacturing Probe Compositions

Provided in certain aspects is a method for designing a plurality of oligonucleotide probes, the method including: identifying a plurality of target nucleic acid fragments of cancer gene exons, introns, untranslated regions and external regions surrounding cancer genes resulting from nucleic acid restriction enzyme cleavage of a nucleic acid sample; and designing a plurality of oligonucleotide probes described herein. In the designed oligonucleotide probes, oligonucleotide probes capable of hybridizing to target nucleic acid fragments of external regions and/or introns often include (i) a set of probe pairs each capable of hybridizing to a target nucleic acid fragment of about 260 consecutive nucleotides or longer in length, and (ii) a set of single oligonucleotide probes each capable of hybridizing to a nucleic acid fragment of about 130 consecutive nucleotides to about 260 consecutive nucleotides in length.

In the designed oligonucleotide probes, the external region and/or intron directed oligonucleotide probes, in the set of oligonucleotide probe pairs and the set of single oligonucleotide probes, often include a polynucleotide (i) about 110 to about 130 consecutive nucleotides in length, (ii) substantially complementary to a subsequence of a nucleic acid fragment, (iii) containing an average GC content of about 40 percent to about 60 percent, and (iv) complementary to a subsequence of a nucleic acid that does not repeat in the nucleic acid fragments. In certain implementations, a design process includes removing from a first set of designed external region and/or intron directed oligonucleotide probes (i) oligonucleotide probes having a GC content less than about 40% and greater than about 60%, and (ii) oligonucleotide probes complementary to a subsequence of a nucleic acid that repeats in the nucleic acid fragments, thereby generating a second set of designed external region and/or intron directed oligonucleotide probes.

In the designed oligonucleotide probes, each of the probe pairs in the set of probe pairs often includes (i) a first oligonucleotide probe comprising a 5′ end about 2 to about 15 consecutive nucleotides from the 5′ end of the fragment, and (ii) a second oligonucleotide probe comprising a 3′ end about 2 to about 15 consecutive nucleotides from the 3′ end of the nucleic acid fragment to which the first oligonucleotide probe of the probe pair is capable of hybridizing. In the designed oligonucleotide probes, each probe in the set of single oligonucleotide probes often includes a 5′ or 3′ end about 2 to about 15 consecutive nucleotides from the 5′ end of a nucleic acid fragment.

Methodology for fragmenting sample nucleic acid in silico, identifying polynucleotides in the fragmented sample nucleic acid in silico, and designing oligonucleotide probes that are complementary to the polynucleotides of the fragmented sample nucleic acid in silico, are known. AGILENT SUREDESIGN™ software and other commercially available software can be utilized to design oligonucleotide probes in silico that are complementary to the polynucleotides of target nucleic acid, for example. Methodology for synthesizing oligonucleotide probes from in silico oligonucleotide probe designs are known, including without limitation phosphite triester synthesis, phosphotriester synthesis and phosphodiester synthesis methodologies.

Preparation and analysis of enriched nucleic acid Provided in certain aspects is a method for nucleic acid enrichment, the method including: subjecting target nucleic acid from a nucleic acid sample to nucleic acid cleavage conditions in which nucleic acid fragments are generated; subjecting the target nucleic acid fragments to linking conditions in which proximity ligated nucleic acid molecules are generated; contacting the proximity ligated nucleic acid molecules with a composition comprising a plurality of oligonucleotide probes described herein under hybridization conditions in which hybridization complexes comprising proximity ligated nucleic acid hybridized to oligonucleotide probes are generated;

isolating the complexes; and analyzing nucleic acid in the complexes.

The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (CDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), small interfering RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides.

A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The term nucleic acid is used interchangeably with locus, gene, CDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil (U). Nucleic acid length or size may be expressed as a number of bases.

Target nucleic acids may be any nucleic acids of interest. Nucleic acids may be polymers of any length composed of deoxyribonucleotides (i.e., DNA bases), ribonucleotides (i.e., RNA bases), or combinations thereof, e.g., 10 bases or longer, 20 bases or longer, 50 bases or longer, 100 bases or longer, 200 bases or longer, 300 bases or longer, 400 bases or longer, 500 bases or longer, 1000 bases or longer, 2000 bases or longer, 3000 bases or longer, 4000 bases or longer, 5000 bases or longer. In certain aspects, nucleic acids are polymers composed of deoxyribonucleotides (i.e., DNA bases), ribonucleotides (i.e., RNA bases), or combinations thereof, e.g., 10 bases or less, 20 bases or less, 50 bases or less, 100 bases or less, 200 bases or less, 300 bases or less, 400 bases or less, 500 bases or less, 1000 bases or less, 2000 bases or less, 3000 bases or less, 4000 bases or less, or 5000 bases or less.

Nucleic acid may be single-stranded or double-stranded. Single-stranded DNA (ssDNA), for example, can be generated by denaturing double-stranded DNA by heating or by treatment with alkali, for example. Accordingly, in some embodiments, ssDNA is derived from double-stranded DNA (dsDNA).

Nucleic acid (e.g., genomic DNA, nucleic acid targets, oligonucleotides, probes, primers) may be described herein as being complementary to another nucleic acid, having a complementarity region, being capable of hybridizing to another nucleic acid, or having a hybridization region. The terms “complementary” or “complementarity” or “hybridization” generally refer to a nucleotide sequence that base-pairs by non-covalent bonds to a region of a nucleic acid. In the canonical Watson-Crick base pairing, adenine (A) forms a base pair with thymine (T), and guanine (G) pairs with cytosine (C) in DNA. In RNA, thymine (T) is replaced by uracil (U). As such, A is complementary to T and G is complementary to C. In RNA, A is complementary to U and vice versa. In a DNA-RNA duplex, A (in a DNA strand) is complementary to U (in an RNA strand). Typically, “complementary” or “complementarity” or “capable of hybridizing” refer to a nucleotide sequence that is at least partially complementary. These terms may also encompass duplexes that are fully complementary such that every nucleotide in one strand is complementary or hybridizes to every nucleotide in the other strand in corresponding positions. In certain instances, a nucleotide sequence may be partially complementary to a target, in which not all nucleotides are complementary to every nucleotide in the target nucleic acid in all the corresponding positions.

The percent identity of two nucleotide sequences can be determined by aligning the sequences for optimal comparison purposes. When the total number of positions is different between the two nucleotide sequences, gaps may be introduced in the sequence of one or both sequences for optimal alignment. The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position. In certain instances, extra or missing bases within a sequence are expressed as gaps in an alignment and may or may not be factored into a percent identity calculation. For example, a percent identity calculation may include a number of mismatches and gaps or may include a number of mismatches only.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, oligonucleotide, or probe, to a nucleic acid molecule having a sequence complementary to the primer, oligonucleotide, or probe compared to hybridization to a nucleic acid molecule not having a complementary sequence. For example, specific hybridization includes the hybridization of a primer, oligonucleotide, or probe to a target nucleic acid sequence that is complementary to the primer, oligonucleotide, or probe. Primer, oligonucleotide, or probe sequences and length can affect hybridization to target nucleic acid sequences. Depending on the degree of mismatch between the primer, oligonucleotide, or probe and target nucleic acid, low, medium or high stringency conditions may be used to effect primer/target, oligonucleotide/target, or probe/target annealing. As used herein, the term “stringent conditions” refers to conditions for hybridization and washing. Methods for hybridization reaction temperature condition optimization are known, and can be found, e.g., in Current Protocols in Molecular Biology, John Wiley & Sons, N.Y., 6.3.1-6.3.6 (1989). Aqueous and non-aqueous methods are described in the aforementioned reference and either can be used. Non-limiting examples of stringent hybridization conditions include, for example, hybridization in 6× sodium chloride/sodium citrate (SSC) at about 45° C., followed by one or more washes in 0.2×SSC, 0.1% SDS at 50° C. Another example of stringent hybridization conditions includes hybridization in 6× sodium chloride/sodium citrate (SSC) at about 45° C., followed by one or more washes in 0.2×SSC, 0.1% SDS at 55° C. A further example of stringent hybridization conditions includes hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions can include 0.5 M sodium phosphate, 7% SDS at 65° C., followed by one or more washes at 0.2×SSC, 1% SDS at 65° C. Stringent hybridization temperatures also can be altered (generally, lowered) with the addition of certain organic solvents, such as formamide for example. Organic solvents such as formamide can reduce the thermal stability of double-stranded polynucleotides, so that hybridization can be performed at lower temperatures, while still maintaining stringent conditions and extending the useful life of heat labile nucleic acids.

In some embodiments, target nucleic acids comprise degraded DNA. Degraded DNA may be referred to as low-quality DNA or highly degraded DNA. Degraded DNA may be highly fragmented and may include damage such as base analogs and abasic sites subject to miscoding lesions and/or intermolecular crosslinking. For example, sequencing errors resulting from deamination of cytosine residues may be present in certain sequences obtained from degraded DNA (e.g., miscoding of C to T and G to A). Nucleic acid may be derived from one or more sources (e.g., a biological sample described herein) by methods known in the art. Any suitable method can be used for isolating, extracting and/or purifying DNA from a biological sample (e.g., from blood or a blood product, tissue, tumor), non-limiting examples of which include methods of DNA preparation, various commercially available reagents or kits, such as DNeasy®, RNeasy®, QIAprep®, QIAquick®, and QIAamp® (e.g., QIAamp® Circulating Nucleic Acid Kit, QiaAmp® DNA Mini Kit or QiaAmp® DNA Blood Mini Kit) nucleic acid isolation/purification kits by Qiagen, Inc. (Germantown, Md); GenomicPrep™ Blood DNA Isolation Kit (Promega, Madison, Wis.); GFX™ Genomic Blood DNA Purification Kit (Amersham, Piscataway, N.J.); DNAzol®, ChargeSwitch®, Purelink®, GeneCatcher® nucleic acid isolation/purification kits by Life Technologies, Inc. (Carlsbad, CA); NucleoMag®, NucleoSpin®, and NucleoBond® nucleic acid isolation/purification kits by Clontech Laboratories, Inc. (Mountain View, CA); the like or combinations thereof. In certain aspects, nucleic acid is isolated from a fixed biological sample, e.g., formalin-fixed, paraffin-embedded (FFPE) tissue. Genomic DNA from FFPE tissue may be isolated using commercially available kits-such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

In some embodiments, nucleic acid is extracted from cells using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized. In some instances, a lysis procedure may include a lysis step with EDTA/Proteinase K, a binding buffer step with high amount of salts (e.g., guanidinium chloride (GuHCl), sodium acetate) and isopropanol, and binding DNA in this solution to silica-based column.

Nucleic acids can include extracellular nucleic acid in certain embodiments. The term “extracellular nucleic acid” as used herein can refer to nucleic acid isolated from a source having substantially no cells and also is referred to as “cell-free” nucleic acid (cell-free DNA, cell-free RNA, or both), “circulating cell-free nucleic acid” (e.g., CCF fragments, ccfDNA) and/or “cell-free circulating nucleic acid.”

Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. In certain aspects, cell-free nucleic acid is obtained from a body fluid sample chosen from whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. As used herein, the term “obtain cell-free circulating sample nucleic acid” includes obtaining a sample directly (e.g., collecting a sample, e.g., a test sample) or obtaining a sample from another who has collected a sample. Extracellular nucleic acid may be a product of cellular secretion and/or nucleic acid release (e.g., DNA release). Extracellular nucleic acid may be a product of any form of cell death, for example. In some instances, extracellular nucleic acid is a product of any form of type I or type II cell death, including mitotic, oncotic, toxic, ischemic, and the like and combinations thereof. Without being limited by theory, extracellular nucleic acid may be a product of cell apoptosis and cell breakdown, which provides basis for extracellular nucleic acid often having a series of lengths across a spectrum (e.g., a “ladder”). In some instances, extracellular nucleic acid is a product of cell necrosis, necropoptosis, oncosis, entosis, pyrotosis, and the like and combinations thereof. In some embodiments, sample nucleic acid from a test subject is circulating cell-free nucleic acid. In some embodiments, circulating cell free nucleic acid is from blood plasma or blood serum from a test subject. In some aspects, cell-free nucleic acid is degraded. In certain aspects, cell-free nucleic acid comprises circulating cancer nucleic acid (e.g., cancer DNA). In certain aspects, cell-free nucleic acid comprises circulating tumor nucleic acid (e.g., tumor DNA). Extracellular nucleic acid can include different nucleic acid species, and therefore is referred to herein as “heterogeneous” in certain embodiments. For example, blood serum or plasma from a person having a tumor or cancer can include nucleic acid from tumor cells or cancer cells (e.g., neoplasia) and nucleic acid from non-tumor cells or non-cancer cells. In some instances, cancer nucleic acid and/or tumor nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, or tumor nucleic acid).

Nucleic acid may be provided for conducting methods described herein with or without processing of the sample(s) containing the nucleic acid. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. In certain examples, small fragments of nucleic acid (e.g., 30 to 500 bp fragments) can be purified, or partially purified, from a mixture comprising nucleic acid fragments of different lengths. In certain examples, nucleosomes comprising smaller fragments of nucleic acid can be purified from a mixture of larger nucleosome complexes comprising larger fragments of nucleic acid. In certain examples, larger nucleosome complexes comprising larger fragments of nucleic acid can be purified from nucleosomes comprising smaller fragments of nucleic acid. In certain examples, cancer cell nucleic acid can be purified from a mixture comprising cancer cell and non-cancer cell nucleic acid.

In certain examples, nucleosomes comprising small fragments of cancer cell nucleic acid can be purified from a mixture of larger nucleosome complexes comprising larger fragments of non-cancer nucleic acid. In some embodiments, nucleic acid is provided for conducting methods described herein without prior processing of the sample(s) containing the nucleic acid. For example, nucleic acid may be analyzed directly from a sample without prior extraction, purification, partial purification, and/or amplification.

In certain instances, cleavage conditions include contacting the sample nucleic acid with a restriction endonuclease, and sometimes the restriction endonuclease cleaves the sample nucleic acid at \hat{ }GATC and G\hat{ }ANTC, where “\hat{ }” represents the cut site. In certain instances, sample nucleic acid is contacted with two or more restriction enzyme types. In certain implementations, linking conditions comprise contacting the nucleic acid fragments with a ligase under conditions in which ends of fragments in proximity are joined. Methodology for preparing proximity ligated nucleic acid is known in the art, and non-limiting examples of such methodology are referred to as Hi-C, 3C, 4C, ChiA-PET and variants thereof (e.g., capture Hi-C), as described additionally herein.

In certain instances, oligonucleotide probes include a capture agent and the complexes are isolated by contacting the complexes with a solid phase comprising a capture agent counterpart that specifically binds to the capture agent under binding conditions. A solid phase sometimes is a plurality of beads, such as magnetic or SEPHAROSE™ beads for example. In certain implementations, (i) a capture agent is selected from biotin, avidin and streptavidin, and (ii) the capture agent counterpart is a molecule that specifically binds to the capture agent and independently is selected from biotin, avidin and streptavidin. Hybridization complexes can be isolated by contacting the complexes with a solid phase that includes a capture agent counterpart under conditions in which the capture agent counterpart of the solid phase specifically binds to a capture agent associated with the oligonucleotide probes of the hybridization complexes, and separating the complexes bound to the solid phase from complexes not bound to the solid phase. Any suitable method for carrying out proximity ligation may be used. For example, a Hi-C method typically includes the following steps: (1) digestion of a chromatin sample with a restriction enzyme (or fragmentation), where a non-limiting example of a chromatin sample is chromatin obtained from solubilized and decompacted FFPE (formalin-fixed paraffin embedded) tissue; (2) labelling the digested ends by filling in the 5′-overhangs with biotinylated nucleotides; and (3) ligating the spatially proximal digested ends, thus preserving spatial-proximal contiguity information. Once spatial-proximal contiguity information is preserved, further steps in a HiC method may include: purifying and enriching biotin-labelled ligation junction fragments, preparing a library from the enriched fragments and sequencing the library. Another example of a proximity ligation method may include the following steps: (1) digestion of a chromatin sample with a restriction enzyme (or fragmentation), where a non-limiting example of a chromatin sample is chromatin obtained from solubilized and decompacted FFPE (formalin-fixed paraffin embedded) tissue; (2) blunting the digested or fragmented ends or omission of the blunting procedure; and (3) ligating the spatially proximal ends, thus preserving spatial-proximal contiguity information. Once spatial-proximal contiguity information is preserved, further steps can include: using size selection to purify and enrich ligated fragments, which represent ligation junction fragments, preparing a library from the enriched fragments and sequencing the library. In some embodiments, proximity ligated nucleic acid molecules are generated in situ (i.e., within a nucleus). For methods that include Capture HiC, a further step is included where ligation products containing certain nucleic acid sequences are enriched using one or more capture probes (see e.g., International Patent Application Publication No. WO 2014/168575), such as a set of oligonucleotide probes described herein.

Processes that include preparing hybridization complexes comprising proximity ligated nucleic acid hybridized to oligonucleotide probes and then isolating the complexes can enrich the relative abundance of polynucleotides in sample nucleic acid complementary to the oligonucleotide probe polynucleotides, which are referred to as “target polynucleotides” or “target nucleic acids” or “target genes” herein. As oligonucleotide probes described herein include polynucleotides complementary to cancer gene introns, exons, promoters, and external regions, such probes are useful for enriching cancer gene polynucleotides in a sample. A subset of hybridization complexes containing proximity ligated nucleic acid hybridized to probes described herein typically is enriched for cancer gene target polynucleotides. Target polynucleotides (e.g., cancer gene oligonucleotides) generally are enriched in a subset of hybridization complexes containing proximity ligated nucleic acid hybridized, or that was hybridized, to the probes, relative to all proximity ligated nucleic acid prepared from a nucleic acid sample. Stated another way, the abundance (e.g., percentage) of target polynucleotides (e.g., cancer gene polynucleotides) generally is greater in the subset of hybridization complexes containing proximity ligated nucleic acid hybridized, or that was hybridized, to the probes, relative to the abundance of target polynucleotides (e.g., percentage) in all proximity ligated nucleic acid prepared from a nucleic acid sample.

Target nucleic acid sometimes is modified as part of a nucleic acid analysis process. A target nucleic acid can be modified to include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, a unique molecular identifier (UMI), the like or combinations thereof. In some embodiments, a nucleic acid or isolated nucleic acid comprises one or more adapters (e.g., sequencing adapters, also known as sequencing adapter oligonucleotides). Sequencing adapters may comprise sequences complementary to flow-cell anchors, and sometimes are utilized to immobilize a nucleic acid to a solid support, such as the inside surface of a flow cell, for example. Adapters and other polynucleotide components described typically are not associated with the nucleic acid in vivo and thereby do not naturally occur with the nucleic acid. In certain instances, analyzing the nucleic acid in the complexes includes sequencing the proximity ligated nucleic acid of the isolated complexes. Target nucleic acid sometimes is modified as part of a sequencing process. In certain implementations, non-naturally occurring oligonucleotides that facilitate sequencing, known as “sequencing adapter oligonucleotides,” are joined to proximity ligated nucleic acid in the oligonucleotide probe-isolated proximity ligated nucleic acid, thereby forming adapter-modified nucleic acid. Adapter-modified nucleic acid is optionally amplified by an amplification process known in the art, and the adapter-modified nucleic acid (or amplified adapter-modified nucleic acid) is then subjected to sequencing conditions to identify the polynucleotide sequence of proximity ligated nucleic acid. Additional aspects of nucleic acid analytical methodology are described herein.

Analysis of nucleic acid from a sample can identify one or more structural variants in the nucleic acid relative to nucleic acid from a reference genome or from another sample in certain instances. Various types of structural variants that can be identified are described herein.

### Samples

Provided herein are methods and compositions for processing and/or analyzing nucleic acid. Nucleic acid utilized in methods and compositions described herein may be isolated from a sample obtained from a subject (e.g., a test subject). A subject can be any living or non-living organism, including but not limited to a human and a non-human animal. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a human. A subject may be a male or female. A subject may be any age (e.g., an embryo, a fetus, an infant, a child, an adult). A subject may be a cancer patient, a patient suspected of having cancer, a patient in remission, a patient with a family history of cancer, and/or a subject obtaining a cancer screen. In some embodiments, a subject is an adult patient. In some embodiments, a subject is a pediatric patient.

A nucleic acid sample may be isolated or obtained from any type of suitable biological specimen or sample (e.g., a test sample). A nucleic acid sample may be isolated or obtained from a single cell, a plurality of cells (e.g., cultured cells), cell culture media, conditioned media, a tissue, an organ, or an organism. In some embodiments, a nucleic acid sample is isolated or obtained from a cell(s), tissue, organ, and/or the like of an animal (e.g., an animal subject). In some instances, a nucleic acid sample may be obtained as part of a diagnostic analysis.

A sample or test sample may be any specimen that is isolated or obtained from a subject or part thereof (e.g., a human subject, a cancer patient, a tumor). Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo; cancer biopsy), celocentesis sample, cells (blood cells, placental cells, embryo or fetal cells, fetal nucleated cells or fetal cellular remnants, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject. A fluid or tissue sample from which nucleic acid is extracted may be acellular (e.g., cell-free). In some embodiments, a fluid or tissue sample may contain cellular elements or cellular remnants. In some embodiments, cancer cells may be included in the sample.

A sample can be a liquid sample. A liquid sample can comprise extracellular nucleic acid (e.g., circulating cell-free DNA). Examples of liquid samples include, but are not limited to, blood or a blood product (e.g., serum, plasma, or the like), urine, cerebrospinal fluid, saliva, sputum, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy). In certain instances, extracellular nucleic acid is analyzed in a liquid biopsy.

In some embodiments, a biological sample may be blood, plasma or serum. The term “blood” encompasses whole blood, blood product or any fraction of blood, such as serum, plasma, buffy coat, or the like as conventionally defined. Blood or fractions thereof often comprise nucleosomes. Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells, B-cells, platelets, and the like). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3 to 40 milliliters, between 5 to 50 milliliters) often is collected and can be stored according to standard procedures prior to or after preparation.

An analysis of nucleic acid found in a subject's blood may be performed using, e.g., whole blood, serum, or plasma. An analysis of tumor or cancer DNA found in a patient's blood, for example, may be performed using, e.g., whole blood, serum, or plasma. Methods for preparing serum or plasma from blood obtained from a subject (e.g., patient; cancer patient) are known. For example, a subject's blood (e.g., patient's blood; cancer patient's blood) can be placed in a tube containing EDTA or a specialized commercial product such as Cell-Free DNA BCT (Streck, Omaha, NE) or Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1, 500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

A sample may be a tumor nucleic acid sample (i.e., a nucleic acid sample isolated from a tumor). The term “tumor” generally refers to neoplastic cell growth and proliferation, whether malignant or benign, and may include pre-cancerous and cancerous cells and tissues. The terms “cancer” and “cancerous” generally refer to the physiological condition in mammals that is typically characterized by unregulated cell growth/proliferation.

In some embodiments, a sample is a tissue sample, a cell sample, a blood sample, or a urine sample. In some embodiments, a sample comprises formalin-fixed, paraffin-embedded (FFPE) tissue. In some embodiments, a sample comprises frozen tissue. In some embodiments, a sample comprises peripheral blood. In some embodiments, a sample comprises blood obtained from bone marrow.

In some embodiments, a sample comprises cells obtained from urine. In some embodiments, a sample comprises cell-free nucleic acid. In some embodiments, a sample comprises one or more tumor cells. In some embodiments, a sample comprises one or more circulating tumor cells. In some embodiments, a sample comprises a solid tumor. In some embodiments, a sample comprises a blood tumor.

### Nucleic Acid Analysis Methodology

Non-limiting examples of processes for analyzing nucleic acid include amplification (e.g., polymerase chain reaction (PCR)), targeted sequencing, microarray, and fluorescence in situ hybridization (FISH), methods that preserves spatial-proximal contiguity information, and methods that generate proximity ligated nucleic acid molecules.

In some embodiments, a nucleic acid analysis comprises nucleic acid amplification. For example, nucleic acids may be amplified under amplification conditions. The term “amplified” or “amplification” or “amplification conditions” generally refer to subjecting a target nucleic acid in a sample to a process that linearly or exponentially generates amplicon nucleic acids having the same or substantially the same nucleotide sequence as the target nucleic acid, or part thereof. In certain embodiments, the term “amplified” or “amplification” or “amplification conditions” refers to a method that comprises a polymerase chain reaction (PCR). Detecting a structural variant (SV) described herein using amplification (e.g., PCR) may include use of primers designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints.

In some embodiments, a nucleic acid analysis comprises fluorescence in situ hybridization (FISH). Fluorescence in situ hybridization (FISH) is a technique that uses fluorescent probes that bind to a nucleic acid sequence with a high degree of sequence complementarity. In certain configurations, fluorescence microscopy may be used to observe where the fluorescent probe is bound to a chromosome. Detecting a structural variant (SV) described herein using fluorescence in situ hybridization (FISH) may include use of probes designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints. Examples of probes useful for identifying a structural variant are provided herein.

In some embodiments, a nucleic acid analysis comprises a microarray (e.g., a DNA microarray, DNA chip, biochip). A DNA microarray is a collection of DNA probes attached to a solid surface. Probes can be short sections of a gene or other genomic DNA element that can hybridize to target nucleic acids in a sample (e.g., under high-stringency conditions). Probe-target hybridization is usually detected and quantified by detection of fluorophore-, silver-, or chemiluminescence-labeled targets to determine presence, absence, and/or relative abundance of target nucleic acid sequences in the sample. Detecting a structural variant (SV) described herein using DNA microarrays may include use of array probes designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints. Examples of array probes useful for identifying a structural variant are provided herein.

In some embodiments, a nucleic acid analysis comprises sequencing (e.g., genome-wide sequencing, targeted sequencing). For targeted sequencing, a target nucleic acid may be amplified (e.g., by PCR with primers specific to the target), enriched using a probe-based approach, where one or more probes hybridize to a target nucleic acid prior to sequencing, or enriched using Cas9-mediated approaches, such as Cas9-guided adapter ligation, as described in Gilpatrick, T. et al., Targeted nanopore sequencing with Cas9-guided adapter ligation, Nature Biotechnology, volume 38, pages 433-438 (2020). Nucleic acid may be sequenced using any suitable sequencing platform including a Sanger sequencing platform, a high throughput or massively parallel sequencing (next generation sequencing (NGS)) platform, or the like, such as, for example, a sequencing platform provided by Illumina® (e.g., HiSeq™, MiSeq™ and/or Genome Analyzer™ sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLID sequencing system); Roche (e.g., 454 GS FLX+ and/or GS Junior sequencing systems); or any other suitable sequencing platform. In some embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In some embodiments, a sequencing process generates short sequencing reads or “short reads.” In some embodiments, the nominal, average, mean or absolute length of short reads sometimes is about 10 continuous nucleotides to about 250 or more contiguous nucleotides. In some embodiments, the nominal, average, mean or absolute length of short reads sometimes is about 50 continuous nucleotides to about 150 or more contiguous nucleotides.

In some embodiments, a nucleic acid analysis comprises a method that preserves spatial-proximal relationships and/or spatial-proximal contiguity information (see e.g., International PCT Application Publication No. WO2019/104034; International PCT Application Publication No. WO2020/106776; International PCT Application Publication No. WO2020236851; Kempfer, R., & Pombo, A. (2019). Methods for mapping 3D chromosome architecture. Nature Reviews Genetics. doi:10.1038/s41576-019-0195-2; and Schmitt, Anthony D.; Hu, Ming; Ren, Bing (2016). Genome-wide mapping and analysis of chromosome architecture. Nature Reviews Molecular Cell Biology. doi:10.1038/nrm.2016.104; each of which is incorporated by reference in its entirety, to the extent permitted by law). Methods that preserve spatial-proximal relationships and/or spatial-proximal contiguity information generally refer to methods that capture and preserve the native spatial conformation exhibited by nucleic acids when associated with proteins as in chromatin and/or as part of a nuclear matrix. Spatial-proximal contiguity information can be preserved by proximity ligation, by solid substrate-mediated proximity capture (SSPC), by compartmentalization with or without a solid substrate or by use of a Tn5 tetramer. Methods that preserve spatial-proximal contiguity information may be based on proximity ligation or may be based on a different principle where spatial proximity is inferred. Methods based on proximity ligation may include, for example, 3C, 4C, 5C, Hi-C, TCC, GCC, TLA, PLAC-seq, HiChIP, ChIA-PET, Capture-C, Capture-HiC, single-cell HiC, sciHiC, single-cell 3C, single-cell methyl-3C, DNAase HiC, Micro-C, Tiled-C, and Low-C. Methods where spatial proximity is inferred based on a principle other than proximity ligation may include, for example, SPRITE, SCSPRITE, Genome Architecture Mapping (GAM), ChIA-Drop, imaging-based approaches using labeled probes and visualization of DNA, and plus/minus sequencing of an imaged sample (e.g. in situ Genome Sequencing (IGS)). In some embodiments, a nucleic acid analysis comprises generating proximity ligated nucleic acid molecules (e.g., using a method described herein). In some embodiments, a nucleic acid analysis comprises amplifying the proximity ligated nucleic acid molecules, e.g., by a suitable amplification process known in the art or described herein. In some embodiments, a nucleic acid analysis comprises sequencing the proximity ligated nucleic acid molecules, e.g., by a suitable sequencing process known in the art or described herein.

In some embodiments, a nucleic acid analysis comprises a method for preparing nucleic acids from particular types of samples that preserves spatial-proximal contiguity information in the sequence of the nucleic acids. Nucleic acid molecules that preserve spatial-proximal contiguity information can fragmented and sequenced using short-read sequencing methods (e.g., Illumina, nucleic acid fragments of lengths approximately 500 bp) or intact molecules that preserve spatial-proximal contiguity information can be sequenced using long-read sequencing (e.g., Illumina, Oxford Nanopore, or others, nucleic acid fragments of lengths approximately 30 K bp or greater). In certain embodiments, a sample can be a fixed sample that is embedded in a material such as paraffin (wax). In some embodiments, a sample can be a formalin fixed sample. In certain embodiments, a sample is formalin-fixed paraffin-embedded (FFPE) sample. In some embodiments, a formalin-fixed paraffin-embedded sample can be a tissue sample or a cell culture sample. In some embodiments, a tissue sample has been excised from a patient and can be diseased or damaged. In some embodiments, a tissue sample is not known to be diseased or damaged. In certain embodiments, a formalin-fixed paraffin-embedded sample can be a formalin-fixed paraffin-embedded section, block, scroll or slide. In certain embodiments, a sample can be a deeply formalin-fixed sample, as described below.

In certain embodiments, a formalin-fixed paraffin-embedded sample is provided on a solid surface and a method of preparing nucleic acid that preserves spatial-proximal contiguity information is performed on the solid surface. In some embodiments, a solid surface is a pathology slide. In some embodiments, additional downstream reactions are also performed on the solid surface.

Those of skill in the art are familiar with methods that can be substituted for steps requiring centrifugation and that achieve a comparable result but are performed on a solid surface. In some embodiments, methods that preserve spatial-proximal contiguity information comprise methods that generate proximity ligated nucleic acid molecules (e.g., using proximity ligation). A proximity ligation method is one in which natively occurring spatially proximal nucleic acid molecules are captured by ligation to generate ligated products. Proximity ligation methods generally capture spatial-proximal contiguity information in the form of ligation products, whereby a ligation junction is formed between two natively spatially proximal nucleic acids. Once the ligation products are formed, the spatial-proximal contiguity information is detected using next generation sequencing, whereby one or more ligation junctions (either from an entire ligation product or fragment of a ligation product) are sequenced (as described herein). With this sequence information, one is informed that the nucleic acid molecules from a given ligation product (or ligation junction) are natively spatially proximal nucleic acids. In some embodiments, reagents that generate proximity ligated nucleic acid molecules can include a restriction endonuclease, a DNA polymerase, a plurality of nucleotides comprising at least one biotinylated nucleotide, and a ligase. In certain embodiments, two or more restriction endonucleases are used. Any suitable method for carrying out proximity ligation may be used, as described herein.

### Structural Variants

Provided herein are methods for detecting the presence or absence of a structural variant in a sample. In certain aspects, provided is a method for detecting the presence or absence of a structural variant in a sample, the method comprising analyzing sample nucleic acid from a subject, wherein the analyzing comprises: generating proximity ligated nucleic acid molecules; contacting the proximity ligated nucleic acid molecules with one or more oligonucleotide probes described herein, thereby generating enriched proximity ligated nucleic acid molecules;

sequencing the enriched proximity ligated nucleic acid molecules, thereby generating sequences of the sample nucleic acid; and determining the presence or absence of a structural variant in the sample nucleic acid from the sequences. In certain instances, the method includes comparing sequences of the sample nucleic acid to sequences of a reference genome to determine the presence or absence of a structural variant. A structural variant may be referred to as a structural variation and/or a chromosomal rearrangement. A structural variant may comprise one or more of a translocation, inversion, insertion, deletion, and duplication. In some embodiments, a structural variant comprises a microduplication and/or a microdeletion. In some embodiments, a structural variant comprises a fusion (e.g., a gene fusion where a portion of a first gene is inserted into a portion of a second gene). Any type of structural variant, whether it be translocation, inversion, insertion, deletion, and/or duplication as described below, can be of any length, and in some embodiments, is about 1 base or base pair (bp) to about 250 megabases (Mb) in length. In some embodiments, a structural variation is about 1 base or base pair (bp) to about 50,000 kilobases (kb) in length (e.g., about 10 bp, 50 bp, 100 bp, 500 bp, 1 kb, 5 kb, 10 kb, 50 kb, 100 kb, 500 kb, 1000 kb, 5000 kb or 10,000 kb in length). A structural variant may be intra-chromosomal (rearrangement of genomic material within a chromosome) or inter-chromosomal (rearrangement of genomic material between two or more chromosomes).

A structural variant may comprise a translocation. A translocation is a genetic event that results in a rearrangement of chromosomal material. Translocations may include reciprocal translocations and Robertsonian translocations. A reciprocal translocation is a chromosome abnormality caused by exchange of parts between non-homologous chromosomes-two detached fragments of two different chromosomes are switched. A Robertsonian translocation occurs when two non-homologous chromosomes become attached, meaning that given two healthy pairs of chromosomes, one of each pair sticks and blends together homogeneously. A gene fusion may be created when a translocation joins two genes that are normally separate. Translocations may be balanced (i.e., in an even exchange of material with no genetic information extra or missing, sometimes with full functionality) or unbalanced (i.e., where the exchange of chromosome material is unequal resulting in extra or missing genes or fragments thereof).

A structural variant may comprise an inversion. An inversion is a chromosome rearrangement in which a segment of a chromosome is reversed end-to-end. An inversion may occur when a single chromosome undergoes breakage and rearrangement within itself. Inversions may be of two types: paracentric and pericentric. Paracentric inversions do not include the centromere, and both breaks occur in one arm of the chromosome. Pericentric inversions include the centromere, and there is a break point in each arm.

A structural variant may comprise an insertion. An insertion may be the addition of one or more nucleotide base pairs into a nucleic acid sequence. An insertion may be a microinsertion (generally a submicroscopic insertion of any length ranging from 1 base to about 10 megabases (e.g., about 1 megabase to about 3 megabases)). In certain embodiments, an insertion comprises the addition of a segment of a chromosome into a genome, chromosome, or segment thereof. In certain embodiments an insertion comprises the addition of an allele, a gene, an intron, an exon, any non-coding region, any coding region, segment thereof or combination thereof into a genome or segment thereof. In certain embodiments an insertion comprises the addition (e.g., insertion) of nucleic acid of unknown origin into a genome, chromosome, or segment thereof. In certain embodiments an insertion comprises the addition (e.g., insertion) of a single base.

A structural variant may comprise a deletion. In certain embodiments, a deletion is a genetic aberration in which a part of a chromosome or a sequence of DNA is missing. A deletion can, in certain embodiments, result in the loss of genetic material. In embodiments, a deletion can be translocated to another portion of the genome (balanced translocation or unbalanced translocation), such as on the same chromosome (same arm of the chromosome or other arm of the chromosome) or on a different chromosome. Any number of nucleotides can be deleted. A deletion can comprise the deletion of one or more entire chromosomes, a segment of a chromosome, an allele, a gene, an intron, an exon, any non-coding region, any coding region, a segment thereof or combination thereof. A deletion can comprise a microdeletion (generally a submicroscopic deletion of any length ranging from 1 base to about 10 megabases (e.g., about 1 megabase to about 3 megabases)). A deletion can comprise the deletion of a single base.

A structural variant may comprise a duplication. In certain embodiments, a duplication is a genetic aberration in which a part of a chromosome or a sequence of DNA is copied and inserted back into the genome. In certain embodiments, a duplication is any duplication of a region of DNA. In some embodiments, a duplication is a nucleic acid sequence that is repeated, often in tandem, within a genome or chromosome. In some embodiments a duplication can comprise a copy of one or more entire chromosomes, a segment of a chromosome, an allele, a gene, an intron, an exon, any non-coding region, any coding region, segment thereof or combination thereof. A duplication can comprise a microduplication (generally a submicroscopic duplication of any length ranging from 1 base to about 10 megabases (e.g., about 1 megabase to about 3 megabases)). A duplication sometimes comprises one or more copies of a duplicated nucleic acid. A duplication may be characterized as a genetic region repeated one or more times (e.g., repeated 1, 2, 3, 4, 5, 6, 7, 8, 9 or 10 times). Duplications can range from small regions (thousands of base pairs) to whole chromosomes in some instances. Duplications may occur as the result of an error in homologous recombination or due to a retrotransposon event. A structural variant may include a plurality of chromosomal rearrangements (e.g., translocations, inversions, insertions, deletions, duplications). For example, a structural variant may include a plurality of intra-chromosomal rearrangements. In certain instances, a structural variant may include a plurality of inter-chromosomal rearrangements. In certain instances, a structural variant may include a plurality of intra-chromosomal rearrangements and inter-chromosomal rearrangements.

A structural variant may be defined according to one or more breakpoints. A breakpoint generally refers to a genomic position (i.e., genomic coordinate) where a structural variant occurs (e.g., translocation, inversion, insertion, deletion, or duplication). A breakpoint may refer to a genomic position where an ectopic portion of genomic material is inserted (e.g., a recipient site for an insertion or a translocation). A breakpoint may refer to a genomic position where a portion of genomic material is deleted (e.g., a donor site for an insertion or a translocation). A breakpoint may refer to a pair of genomic positions (i.e., genomic coordinates) that have become flanking (i.e., adjacent) to one another as a result of a structural variant (e.g., translocation, inversion, insertion, deletion, or duplication). A breakpoint may be defined in terms of a position or positions in a reference genome. A breakpoint may be defined in terms of a position or positions in a human reference genome (e.g., HG38 human reference genome). Generally, genomic positions discussed herein are in reference to an HG38 human reference genome, and corresponding and/or equivalent positions in any other human reference genome are contemplated herein. A breakpoint may be defined in terms mapping to a position or positions in a reference genome. A breakpoint may be defined in terms of mapping to a position or positions in a human reference genome (e.g., HG38 human reference genome). A breakpoint may map to a position in a reference genome when a nucleic acid sequence located upstream, downstream, or spanning the breakpoint aligns with a corresponding sequence in a reference genome. Any suitable mapping method (e.g., process, algorithm, program, software, module, the like or combination thereof) can be used and certain aspects of mapping processes are described hereafter.

Mapping a nucleic acid sequence may comprise mapping one or more nucleic acid sequence reads (e.g., sequence information from a fragment whose physical genomic position is unknown), which can be performed in a number of ways, and often comprises alignment of the obtained sequence reads with a matching sequence in a reference genome. In such alignments, sequence reads generally are aligned to a reference sequence and those that align are designated as being “mapped”, “a mapped sequence read” or “a mapped read”.

The terms “aligned”, “alignment”, or “aligning” generally refer to two or more nucleic acid sequences that can be identified as a match (e.g., 100% identity) or partial match. Alignments can be done manually or by a computer (e.g., a software, program, module, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (e.g., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch (i.e., a base not correctly paired with its canonical Watson-Crick base partner (e.g., A or T incorrectly paired with C or G). In some embodiments, an alignment comprises 1, 2, 3, 4 or 5 mismatches. Two or more sequences can be aligned using either strand. In certain embodiments a nucleic acid sequence is aligned with the reverse complement of another nucleic acid sequence. In certain instances, extra or missing bases within a sequence are expressed as gaps in an alignment and may or may not be factored into a percent identity calculation. For example, a percent identity calculation may include a number of mismatches and gaps or may include a number of mismatches only.

Various computational methods can be used to map and/or align sequence reads to a reference genome. Non-limiting examples of computer algorithms that can be used to align sequences include, without limitation, BLAST, BLITZ, FASTA, BOWTIE 1, BOWTIE 2, ELAND, MAQ, PROBEMATCH, SOAP or SEQMAP, or variations thereof or combinations thereof. In some embodiments, sequence reads can be aligned with reference sequences and/or sequences in a reference genome. In some embodiments, the sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search the identified sequences against a sequence database.

A structural variant may be defined in terms of a receiving site and a donor site. A receiving site may be referred to as a first partner or “partner 1” and a donor site may be referred to as a second partner or “partner 2.” In some embodiments, a structural variant may be defined in terms of comprising an ectopic portion of genomic DNA (i.e., a portion of genomic DNA at a receiving site from a different region of a chromosome or from a different chromosome). The ectopic portion may be referred to as a donor portion.

In some embodiments, a structural variant may comprise an ectopic portion of genomic DNA (i.e., a portion of genomic DNA at a receiving site from a different region of a chromosome or from a different chromosome). The ectopic portion may be referred to as a donor portion. If the ectopic portion (donor portion) is from the same chromosome as the structural variant, the ectopic portion may be from a location outside of the position ranges provided herein for certain structural variants. The ectopic portion may comprise genomic DNA from a genomic coordinate window provided herein, or part thereof. The ectopic portion may comprise genomic DNA from a genomic coordinate window provided herein, or part thereof, and may further comprise genomic DNA from a region outside of a genomic coordinate window provided herein.

In some embodiments, an ectopic portion of genomic DNA is characterized by its location (e.g., observed location for a given sample or samples) at a receiving site (e.g., at a structural variant site). In some embodiments, an ectopic portion is characterized by its location (e.g., observed location for a given sample samples) relative to a coding region of a gene and/or cancer gene. A coding region of a gene and/or cancer gene generally refers to a part of the gene and/or cancer gene that is transcribed and translated into protein (i.e., the sum total of its exons). In some embodiments, an ectopic portion is within a coding region of a gene and/or cancer gene. In some embodiments, an ectopic portion is not within a coding region of a gene and/or cancer gene. For example, an ectopic portion may be located in an intronic region, an intergenic region, within another gene or in an external region, as that term is defined herein. In some embodiments, an ectopic portion is located at a position in proximity to a coding region for a gene and/or cancer gene. The term “in proximity” may refer to spatial proximity and/or linear proximity.

Spatial proximity generally refers to three-dimensional chromatin proximity, which may be assessed according to a method that preserves spatial-proximal relationships, such as a method described herein or any suitable method known in the art. An ectopic portion may be located at a position in spatial proximity to a coding region for a gene and/or cancer gene when an ectopic portion and a gene and/or cancer gene (or a fragment thereof) are ligated in a proximity ligation assay or are bound by a common solid phase in a solid substrate-mediated proximity capture (SSPC) assay, for example.

Linear proximity generally refers to a linear base-pair distance, which may be assessed according to mapped distances in a reference genome, for example. Linear proximity distance may be provided as a distance between a 5′ or 3′ end of an ectopic portion and a 5′ or 3′ end of a gene and/or exon. An ectopic portion may be located at a position in linear proximity to a coding region of a gene and/or cancer gene when the ectopic portion is within about 1,000 base pairs, about 2,000 base pairs, about 3,000 base pairs, about 4,000 base pairs, about 5,000 base pairs, about 10,000 base pairs, about 20,000 base pairs, about 30,000 base pairs, about 40,000 base pairs, about 50,000 base pairs, about 60,000 base pairs, about 70,000 base pairs, about 80,000 base pairs, about 90,000 base pairs, about 100,000 base pairs, about 200,000 base pairs, about 300,000 base pairs, about 400,000 base pairs, about 500,000 base pairs, about 600,000 base pairs, about 700,000 base pairs, about 800,000 base pairs, about 900,000 base pairs, or about 1,000,000 base pairs of a coding region of a gene and/or cancer gene.

A structural variant may be associated with one or more genes. For example, a structural variant may be associated with one or more cancer genes. An cancer gene is a gene that, when altered, is associated with cancer. Alterations may include mutations, structural variants, copy number variations, and the like and combinations thereof. Alterations may be located within a gene and/or cancer gene (i.e., intragenic) or outside of/adjacent to a gene and/or cancer gene (i.e., intergenic, extragenic). For structural variants, the terms “outside of” and “adjacent to,” as used herein in reference to a structural variant being outside of or adjacent to a gene generally means that a breakpoint of a structural variant is not within the gene. The structural variant can contain the gene, such as an inversion of the gene, an insertion of the gene, a duplication of the gene, or the like, or can contain a portion of the gene. In certain aspects, the structural variant may not include the gene, i.e., the structural variant does not contain the gene, insertion, inversion, duplication or any portion thereof.

In certain instances, alterations may be located within a different gene. Alterations may be located in a portion of genomic DNA that is proximal to a gene and/or cancer gene (e.g., within a certain linear proximity and/or within a certain spatial proximity). Alterations may affect expression of a gene and/or cancer gene (e.g., increased expression, decreased expression, no expression, constitutive expression). Alterations may affect the function of a protein encoded by a gene and/or cancer gene (e.g., increased function, decreased function, loss-of-function, gain-of-function, constitutive function, change in function).

In some embodiments, a structural variant and/or breakpoint of a structural variant is within a gene (e.g., within an intron and/or exon of a gene (e.g., a cancer gene)). In some embodiments, a structural variant and/or breakpoint of a structural variant is outside of a gene (e.g., within an external region). In some embodiments, a structural variant and/or breakpoint of a structural variant is adjacent to a gene (e.g., within an external region). Thus, in some embodiments, a structural variant and/or a breakpoint for a structural variant is not within a gene (e.g., a cancer gene). In certain instances, a structural variant and/or breakpoint of a structural variant (e.g., an intergenic structural variant) may be defined in terms of linear distance to a gene (e.g., a cancer gene). Linear distance may be measured from the 5′ end of a gene and/or a 3′ end of a gene. In some embodiments, a structural variant and/or a breakpoint for a structural variant may be located at least about 1 kb to about 700 kb from the 5′ end or 3′ end of a gene. For example, a structural variant and/or a breakpoint for a structural variant may be located at least about 1 kb, 2 kb, 3 kb, 4 kb, 5 kb, 10 kb, 20 kb, 30 kb, 40 kb, 50 kb, 60 kb, 70 kb, 80 kb, 90 kb, 100 kb, 200 kb, 300 kb, 400 kb, 500 kb, 600 kb, or 700 kb from the 5′ end or 3′ end of a gene.

### Kits

Provided in certain embodiments are kits. A kit may include any components and compositions described herein (e.g., oligonucleotide probes, nucleic acids, primers, vectors, enzymes) useful for performing any of the methods described herein, in any suitable combination. Kits may further include any reagents, buffers, or other components useful for carrying out any of the methods described herein. A kit sometimes includes one or more isolated enzymes. In certain instances, a kit can include one or more isolated restriction enzymes suitable for cleaving sample nucleic acid into target sample nucleic acid fragments. A kit sometimes includes an isolated ligase suitable for ligating cleaved sample nucleic acid fragments that are in proximity to one another after cleavage. In certain implementations, a kit includes an isolated polymerase, such as a polymerase useful for conducting an amplification process. A kit sometimes includes one or more oligonucleotide primers useful for conducting an amplification process, and sometimes includes one or more adapter oligonucleotides useful for conducting a sequencing process. Certain enzymes (e.g., isolated enzymes) typically are not associated with polynucleotides of oligonucleotide probes and/or sample nucleic acid in vivo and do not naturally occur together.

A kit in certain implementations includes a solid phase comprising a capture agent suitable for specifically binding to a capture agent counterpart incorporated in oligonucleotide probes and thereby suitable for capturing, isolating, purifying and/or enriching probe hybridization complexes. A solid phase sometimes is a plurality of beads. In certain implementations, a capture agent is selected from biotin, avidin and streptavidin, and the capture agent counterpart is a molecule that specifically binds to the capture agent and independently is selected from biotin, avidin and streptavidin.

Components of a kit may be present in separate containers, or multiple components may be present in a single container.

Suitable containers include a single tube (e.g., vial), one or more wells of a plate (e.g., a 96-well plate, a 384-well plate, and the like), and the like.

Kits may also comprise instructions for performing one or more methods described herein and/or a description of one or more components described herein. For example, a kit may include instructions for using oligonucleotide probes and other components described herein. Instructions and/or descriptions may be in printed form and may be included in a kit insert. In some embodiments, instructions and/or descriptions are provided as an electronic storage data file present on a suitable computer readable storage medium, e.g., portable flash drive, DVD, CD-ROM, diskette, and the like. A kit also may include a written description of an internet location that provides such instructions or descriptions.

### Certain Implementations

Following are non-limiting examples of certain implementations of the technology.

A1. A composition, comprising: a plurality of oligonucleotide probes capable of hybridizing to target nucleic acid fragments of external regions surrounding cancer genes resulting from nucleic acid fragmentation of a nucleic acid sample.

A.1.1. A composition, comprising: a plurality of oligonucleotide probes capable of hybridizing to target nucleic acid fragments of external regions surrounding cancer genes resulting from nucleic acid fragmentation of a nucleic acid sample, wherein:
a plurality of external region directed oligonucleotide probes capable of hybridizing to target nucleic acid fragments from external regions comprising (i) a set of probe pairs each capable of hybridizing to a target nucleic acid fragment of about 260 consecutive nucleotides or longer, and (ii) a set of single oligonucleotide probes each capable of hybridizing to a nucleic acid fragment of about 130 consecutive nucleotides to about 260 consecutive nucleotides in length;
the external region directed oligonucleotide probes in the set of oligonucleotide probe pairs and the set of single oligonucleotide probes comprise a polynucleotide (i) about 110 to about 130 consecutive nucleotides in length, (ii) substantially complementary to a subsequence of a nucleic acid fragment, (iii) containing an average GC content of about 40 percent to about 60 percent, and (iv) complementary to a subsequence of a nucleic acid that does not repeat in the nucleic acid fragments;
each of the oligonucleotide probe pairs in the set of oligonucleotide probe pairs comprises (i) a first oligonucleotide probe comprising a 5′ end about 2 to about 15 consecutive nucleotides from the 5′ end of the fragment, and (ii) a second oligonucleotide probe comprising a 3′ end about 2 to about 15 consecutive nucleotides from the 3′ end of the nucleic acid fragment to which the first oligonucleotide probe of the probe pair is capable of hybridizing; and
each oligonucleotide probe in the set of single oligonucleotide probes comprises a 5′ or 3′ end about 2 to about 15 consecutive nucleotides from the 5′ end of a nucleic acid fragment.

A1.2 A composition, comprising: a plurality of oligonucleotide probes capable of hybridizing to target nucleic acid fragments of cancer gene exons, introns, promoters, and external regions surrounding cancer genes resulting from nucleic acid fragmentation of a nucleic acid sample, wherein:
a plurality of external region directed oligonucleotide probes capable of hybridizing to target nucleic acid fragments from external regions and/or introns comprises (i) a set of probe pairs each capable of hybridizing to a target nucleic acid fragment of about 260 consecutive nucleotides or longer, and (ii) a set of single oligonucleotide probes each capable of hybridizing to a nucleic acid fragment of about 130 consecutive nucleotides to about 260 consecutive nucleotides in length;
the external region and/or intron directed oligonucleotide probes in the set of oligonucleotide probe pairs and the set of single oligonucleotide probes comprise a polynucleotide (i) about 110 to about 130 consecutive nucleotides in length, (ii) substantially complementary to a subsequence of a nucleic acid fragment, (iii) containing an average GC content of about 40 percent to about 60 percent, and (iv) complementary to a subsequence of a nucleic acid that does not repeat in the nucleic acid fragments;
each of the oligonucleotide probe pairs in the set of oligonucleotide probe pairs comprises (i) a first oligonucleotide probe comprising a 5′ end about 2 to about 15 consecutive nucleotides from the 5′ end of the fragment, and (ii) a second oligonucleotide probe comprising a 3′ end about 2 to about 15 consecutive nucleotides from the 3′ end of the nucleic acid fragment to which the first oligonucleotide probe of the probe pair is capable of hybridizing; and
each oligonucleotide probe in the set of single oligonucleotide probes comprises a 5′ or 3′ end about 2 to about 15 consecutive nucleotides from the 5′ end of a nucleic acid fragment.

A2. The composition of embodiment A1, wherein the oligonucleotide probes in the set of oligonucleotide probe pairs and the set of single oligonucleotide probes are capable of hybridizing to nucleic acid fragments from 20 or more cancer genes.

A3. The composition of embodiment A1, wherein the oligonucleotide probes in the set of oligonucleotide probe pairs and the set of single oligonucleotide probes are capable of hybridizing to nucleic acid fragments from 20 or more cancer genes.

A4. The composition of embodiment A1, wherein the oligonucleotide probes in the set of oligonucleotide probe pairs and the set of single oligonucleotide probes are capable of hybridizing to nucleic acid fragments from 75 or more cancer genes, 100 or more cancer genes, 120 or more cancer genes, or 130 or more cancer genes.

A4.1 The composition of any of embodiments A1-A4, wherein any one cancer gene, or the 20 or more, 50 or more, 75 or more, 100 or more, 120 or more or 130 or more cancer genes or subset thereof, is/are selected from the group of cancer genes listed in Appendix 1 or Appendix 2.

A5. RESERVED

A6. RESERVED

A7. RESERVED

A8. RESERVED

A9. RESERVED

A10. RESERVED

A11. RESERVED

A12. The composition of embodiment A1, wherein the promoter regions comprise a 5′ end about 500 consecutive nucleotides to about 1500 consecutive nucleotides from a 5′ end of each cancer gene coding region.

A13. The composition of any one of embodiments A1-A12, wherein the polynucleotide of each of the oligonucleotide probes in the set of oligonucleotide probe pairs and the set of single oligonucleotide probes consists of about 110 to about 130 consecutive nucleotides.

A14. The composition of any one of embodiments A1-A13, wherein the polynucleotide of each of the oligonucleotide probes in the set of oligonucleotide probe pairs and the set of single oligonucleotide probes consists of about 120 consecutive nucleotides.

A15. The composition of any one of embodiments A1-A13, wherein the polynucleotide of each of the oligonucleotide probes in the set of oligonucleotide probe pairs and the set of single oligonucleotide probes is complementary to a subsequence of a nucleic acid fragment.

A16. The composition of embodiment A15, wherein the polynucleotide or each of the oligonucleotide probes in the set of oligonucleotide probe pairs and the set of single oligonucleotide probes is 100% complementary to the minus strand of Genome Reference Consortium Human Build 38 (GRCH38).

A16.1. The composition of embodiment A15, wherein the polynucleotide of each of the oligonucleotide probes in the set of oligonucleotide probe pairs and the set of single oligonucleotide probes is 100% complementary to a corresponding portion of the plus strand of Genome Reference Consortium Human Build 38 (GRCH38).

A16.2. The composition of embodiment A15, wherein the composition comprises a mixture of (i) polynucleotides of oligonucleotide probes that are 100% complementary to a corresponding portion of the minus strand of Genome Reference Consortium Human Build 38 (GRCH38) and (ii) polynucleotides or oligonucleotide probes that are 100% complementary to a corresponding portion of the minus strand of Genome Reference Consortium Human Build 38 (GRCH38).

A17. The composition of any one of embodiments A1-A16.2, wherein each of the oligonucleotide probes in the set of oligonucleotide probe pairs and the set of single oligonucleotide probes is capable of hybridizing to a fragment under hybridization conditions of moderate stringency and/or high stringency.

A18. The composition of any one of embodiments A1-A17, wherein the polynucleotides of the oligonucleotide probes in the composition are capable of hybridizing to sample nucleic acid fragments having an average fragment size of about 180 consecutive nucleotides to about 200 consecutive nucleotides.

A19. The composition of any one of embodiments A1-A18, wherein the polynucleotides of the oligonucleotide probes in the composition are capable of hybridizing to target nucleic acid fragments having an average GC content of about 40% to about 45%.

A20. The composition of embodiment A19, wherein the average GC content is about 43%.

A21. The composition of any one of embodiments A1-A20, wherein the polynucleotides of the oligonucleotide probes in the composition are capable of hybridizing to target nucleic acid fragments of about 1% to about 97%.

A22. The composition of any one of embodiments A1-A21, wherein the oligonucleotide probes in the composition comprise a biotin molecule.

A23. The composition of any one of embodiments A1-A22, wherein the polynucleotide of each of the oligonucleotide probes comprises RNA.

A24. The composition of any one of embodiments A1-A22, wherein the polynucleotide of each of the oligonucleotide probes consists of RNA.

A25. The composition of any one of embodiments A1-A24, wherein the target nucleic acid fragments result from nucleic acid restriction enzyme cleavage by restriction enzymes cutting at \hat{ }GATC and G\hat{ }ANTC, where “\hat{ }” represents the cut site on the positive DNA strand.

A26. The composition of embodiment A25, wherein the restriction enzyme cleavage is by two restriction enzymes.

A27. The composition of any one of embodiments A1-A26, wherein the plurality of oligonucleotide probes capable of hybridizing to target nucleic acid fragments from external regions and/or introns are not capable of hybridizing to tiled subsequences of a target nucleic acid fragment.

A28. The composition of any one of embodiments A1-A27, wherein the plurality of oligonucleotide probes capable of hybridizing to target nucleic acid fragments from external regions and/or introns does not contain a probe capable of hybridizing to an end of a first target nucleic acid fragment and to an end of a second target nucleic acid fragment.

A29. The composition of any one of embodiments A1-A28, wherein the first oligonucleotide probe and the second oligonucleotide probe in each of the oligonucleotide probe pairs do not hybridize to regions of a target nucleic acid fragment that overlap.

A30. The composition of any one of embodiments A1-A29, wherein the plurality of oligonucleotide probes capable of hybridizing to target nucleic acid fragments from external or intronic regions does not contain a probe capable of hybridizing to target nucleic acid fragment having a length of less than 130 consecutive nucleotides.

A31. The composition of any one of embodiments A1-A30, wherein the oligonucleotide probes of the plurality of oligonucleotide probes capable of hybridizing to target nucleic acid fragments from external or intronic regions capable of hybridizing to a target nucleic acid fragment are not capable of hybridizing to contiguous, non-overlapping regions of the nucleic acid fragment.

A32. The composition of any one of embodiments A1-A31, wherein each oligonucleotide probe of a plurality of oligonucleotide probes capable of hybridizing to target nucleic acid fragments of exons and promoters is capable of hybridizing to a region spanning about 100 to about 500 consecutive nucleotides from the 5′ end of each target nucleic acid fragment or to a region spanning about 100 to about 500 consecutive nucleotides from the 3′ end of each target nucleic acid fragment.

A33. The composition of any one of embodiments A1-A32, wherein the oligonucleotide probes of the plurality of oligonucleotide probes capable of hybridizing to target nucleic acid fragments of exons and promoters are capable of hybridizing to a region at a first region spanning about 350 consecutive nucleotides from the 5′ end of each target nucleic acid fragment or to a region spanning about 350 consecutive nucleotides from the 3′ end of each target nucleic acid fragment.

A34. The composition of any one of embodiments A1-A33, wherein the oligonucleotide probes of the plurality of oligonucleotide probes capable of hybridizing to target nucleic acid fragments of exons and promoters capable of hybridizing to a target nucleic acid fragment are capable of hybridizing to contiguous, non-overlapping regions of the nucleic acid fragment.

A35. The composition of any one of embodiments A1-A34, wherein the oligonucleotide probes of the plurality of oligonucleotide probes capable of hybridizing to target nucleic acid fragments of exons and promoters are not restricted to a GC content percentage range or threshold.

A36. The composition of any one of embodiments A1-A35, comprising about 20,000 or more oligonucleotide probes.

A37. The composition of embodiment A36, comprising about 21,000 or more oligonucleotide probes.

A38. The composition of any one of embodiments A1-A37, comprising about 15,000 to about 25,000 oligonucleotide probes.

A39. The composition of any one of embodiments A1-A37, comprising about 18,000 to about 24,000 oligonucleotide probes.

A40. The composition of any one of embodiments A1-A37, comprising about 19,000 to about 23,000 oligonucleotide probes.

A41. The composition of any one of embodiments A1-A37, comprising about 20,000 to about 22,000 oligonucleotide probes.

A42. The composition of any one of embodiments A1-A41, comprising about 4,500 to about 8,000 unique oligonucleotide probes.

A43. The composition of any one of embodiments A1-A41, comprising about 5,000 to about 7,500 unique oligonucleotide probes.

A44. The composition of any one of embodiments A1-A41, comprising about 5,500 to about 7,000 unique oligonucleotide probes.

A45. The composition of any one of embodiments A1-A41, comprising about 6,600 unique oligonucleotide probes.

A46. The composition of any one of embodiments A1-A45, wherein the external region comprises padding selected from the group of: 0 kb, + and/or − approximately 50 kb, + and/or − approximately 100 kb, + and/or − approximately 250 kb, + and/or minus approximately 500 kb, + and/or minus approximately 1000 kb, + and/or minus approximately 2000 kb, + and/or minus approximately 5000 kb.

A47. The composition of any one of embodiments A1-A46, wherein a target cancer gene less than approximately 100 kb has extension regions added before padding is applied.

A48. The composition of any one of embodiments A1-A47, wherein if an upstream target gene upstream and a downstream target gene are adjacent or in close proximity in linear sequence on a chromosome, padding will only be applied upstream of the upstream target gene's promoter and downstream of the downstream gene's downstream most stop site.

B1. A method for nucleic acid enrichment, comprising:
subjecting target nucleic acid from a nucleic acid sample to nucleic acid cleavage conditions in which nucleic acid fragments are generated;
subjecting the target nucleic acid fragments to linking conditions in which proximity ligated nucleic acid molecules are generated;
contacting the proximity ligated nucleic acid molecules with a composition comprising a plurality of oligonucleotide probes of any one of embodiments A1-A47 under hybridization conditions in which hybridization complexes comprising proximity ligated nucleic acid hybridized to oligonucleotide probes are generated;
isolating the complexes; and
analyzing nucleic acid in the complexes.

B2. The method of embodiment B1, wherein the cleavage conditions comprise contacting the sample nucleic acid with a restriction endonuclease.

B3. The method of embodiment B2, wherein the restriction endonuclease cleaves the sample nucleic acid at \hat{ }GATC and G\hat{ }ANTC, where “\hat{ }” represents the cut site.

B4. The method of embodiment B2 or B3, wherein the sample nucleic acid is contacted with two or more restriction enzyme types.

B5. The method of any one of embodiments B1-B4, wherein the linking conditions comprise contacting the nucleic acid fragments with a ligase under conditions in which ends of fragments in proximity are joined.

B6. The method of any one of embodiments B1-B5, wherein the oligonucleotide probes comprise biotin and the complexes are isolated by contacting the complexes with a solid phase comprising avidin or streptavidin.

B7. The method of any one of embodiments B1-B6, wherein the analyzing the nucleic acid in the complexes comprises sequencing the proximity ligated nucleic acid.

C1. A kit comprising a composition of any one of embodiments A1-A47.

C2. A kit comprising instructions or a link to instructions describing the method of any one of embodiments B1-B7.

C3. A kit of embodiment C1 or C2, comprising one or more isolated restriction enzymes.

C4. The kit of embodiment C3, wherein the one or more isolated restriction enzymes are suitable for cleaving sample nucleic acid into target sample nucleic acid fragments.

C5. The kit of any one of embodiments C1-C4, comprising an isolated ligase enzyme.

C6. The kit of embodiment C5, wherein the isolated ligase enzyme is suitable for ligating cleaved sample nucleic acid fragments that are in proximity to one another.

C7. The kit of any one of embodiments C1-C6, comprising a solid phase.

C8. The kit of embodiment C7, wherein the oligonucleotide probes comprise a capture agent and the solid phase comprises a capture agent counterpart suitable for binding to a capture agent of the oligonucleotide probes.

C9. The kit of embodiment C8, wherein the capture agent and the capture agent counterpart independently are selected from biotin, avidin and streptavidin.

C10. The kit of any one of embodiments C7-C9, wherein the solid phase is beads.

D1. A method for designing a plurality of oligonucleotide probes, comprising:
identifying a plurality of target nucleic acid fragments of external regions surrounding cancer genes resulting from nucleic acid fragmentation of a nucleic acid sample;
designing a plurality of external region directed oligonucleotide probes capable of hybridizing to target nucleic acid fragments of external regions, comprising (i) a set of probe pairs each capable of hybridizing to a target nucleic acid fragment of about 260 consecutive nucleotides or longer in length, and (ii) a set of single oligonucleotide probes each capable of hybridizing to a nucleic acid fragment of about 130 consecutive nucleotides to about 260 consecutive nucleotides in length; wherein:
   the external region directed oligonucleotide probes in the set of oligonucleotide probe pairs and the set of single oligonucleotide probes comprise a polynucleotide (i) about 110 to about 130 consecutive nucleotides in length, (ii) substantially complementary to a subsequence of a nucleic acid fragment, (iii) containing an average GC content of about 40 percent to about 60 percent, and (iv) complementary to a subsequence of a nucleic acid that does not repeat in the nucleic acid fragments;
each of the oligonucleotide probe pairs in the set of oligonucleotide probe pairs comprises (i) a first oligonucleotide probe comprising a 5′ end about 2 to about 15 consecutive nucleotides from the 5′ end of the fragment, and (ii) a second oligonucleotide probe comprising a 3′ end about 2 to about 15 consecutive nucleotides from the 3′ end of the nucleic acid fragment to which the first oligonucleotide probe of the probe pair is capable of hybridizing; and each oligonucleotide probe in the set of single oligonucleotide probes comprises a 5′ or 3′ end about 2 to about 15 consecutive nucleotides from the 5′ end of a nucleic acid fragment.

D1.1 A method for designing a plurality of oligonucleotide probes, comprising:
identifying a plurality of target nucleic acid fragments of cancer gene exons, introns, and promoters, and external regions surrounding cancer genes resulting from nucleic acid fragmentation of a nucleic acid sample;
designing a plurality of external region directed oligonucleotide probes capable of hybridizing to target nucleic acid fragments of external regions, comprising (i) a set of probe pairs each capable of hybridizing to a target nucleic acid fragment of about 260 consecutive nucleotides or longer in length, and (ii) a set of single oligonucleotide probes each capable of hybridizing to a nucleic acid fragment of about 130 consecutive nucleotides to about 260 consecutive nucleotides in length; wherein:
   the external region directed oligonucleotide probes in the set of oligonucleotide probe pairs and the set of single oligonucleotide probes comprise a polynucleotide (i) about 110 to about 130 consecutive nucleotides in length, (ii) substantially complementary to a subsequence of a nucleic acid fragment, (iii) containing an average GC content of about 40 percent to about 60 percent, and (iv) complementary to a subsequence of a nucleic acid that does not repeat in the nucleic acid fragments;
each of the oligonucleotide probe pairs in the set of oligonucleotide probe pairs comprises (i) a first oligonucleotide probe comprising a 5′ end about 2 to about 15 consecutive nucleotides from the 5′ end of the fragment, and (ii) a second oligonucleotide probe comprising a 3′ end about 2 to about 15 consecutive nucleotides from the 3′ end of the nucleic acid fragment to which the first oligonucleotide probe of the probe pair is capable of hybridizing; and each oligonucleotide probe in the set of single oligonucleotide probes comprises a 5′ or 3′ end about 2 to about 15 consecutive nucleotides from the 5′ end of a nucleic acid fragment.

D2. The method of embodiment D1, wherein the oligonucleotide probes in the set of oligonucleotide probe pairs and the set of single oligonucleotide probes are capable of hybridizing to nucleic acid fragments from 20 or more cancer genes.

D3. The method of embodiment D1, wherein the oligonucleotide probes in the set of oligonucleotide probe pairs and the set of single oligonucleotide probes are capable of hybridizing to nucleic acid fragments from 50 or more cancer genes.

D4. The method of embodiment D1, wherein the oligonucleotide probes in the set of oligonucleotide probe pairs and the set of single oligonucleotide probes are capable of hybridizing to nucleic acid fragments from 75 or more cancer genes, 100 or more cancer genes, 120 or more cancer genes, or 130 or more cancer genes.

D4.1 The composition of any of embodiments D1 to D4, wherein any one cancer gene, 75 or more cancer genes, 100 or more cancer genes, 120 or more cancer genes, or 130 or more cancer genes or a subset thereof, is/are selected from the group of cancer genes listed in Appendix 1 or Appendix 2.

D5. The method of any one of embodiments D1-D4, wherein the promoter region comprises (i) a nucleic acid region extending 1500 bp in the 5′ direction from the most upstream transcription start site of an cancer gene and (ii) a nucleic acid region extending 500 bp in the 3′ direction from the most upstream transcription start site of an cancer gene.

D6. RESERVED

D7. RESERVED

D8. RESERVED

D9. RESERVED

D10. RESERVED

D11. RESERVED

D12. RESERVED

D13. The method of any one of embodiments D1-D12, wherein the polynucleotide of each of the oligonucleotide probes in the set of oligonucleotide probe pairs and the set of single oligonucleotide probes consists of about 110 to about 130 consecutive nucleotides.

D14. The method of any one of embodiments D1-D13, wherein the polynucleotide of each of the oligonucleotide probes in the set of oligonucleotide probe pairs and the set of single oligonucleotide probes consists of about 120 consecutive nucleotides.

D15. The method of any one of embodiments D1-D13, wherein the polynucleotide of each of the oligonucleotide probes in the set of oligonucleotide probe pairs and the set of single oligonucleotide probes is complementary to a subsequence of a nucleic acid fragment.

D16. The method of embodiment D15, wherein the polynucleotide of each of the oligonucleotide probes in the set of oligonucleotide probe pairs and the set of single oligonucleotide probes is 100% complementary to the minus strand of Genome Reference Consortium Human Build 38 (GRCH38).

D16.1. The method of embodiment D15, wherein the polynucleotide of each of the oligonucleotide probes in the set of oligonucleotide probe pairs and the set of single oligonucleotide probes is 100% complementary to a corresponding portion of the plus strand of Genome Reference Consortium Human Build 38 (GRCH38).

D16.2. The method of embodiment D15, wherein the plurality of oligonucleotide probes comprises a mixture of (i) polynucleotides of oligonucleotide probes that are 100% complementary to a corresponding portion of the minus strand of Genome Reference Consortium Human Build 38 (GRCH38) and (ii) polynucleotides or oligonucleotide probes that are 100% complementary to a corresponding portion of the minus strand of Genome Reference Consortium Human Build 38 (GRCH38).

D17. The method of any one of embodiments D1-D16.2, wherein each of the oligonucleotide probes in the set of oligonucleotide probe pairs and the set of single oligonucleotide probes is capable of hybridizing to a fragment under hybridization conditions of moderate stringency and/or high stringency.

D18. The method of any one of embodiments D1-D17, wherein the polynucleotides of the oligonucleotide probes in the composition are capable of hybridizing to sample nucleic acid fragments having an average fragment size of about 180 consecutive nucleotides to about 200 consecutive nucleotides.

D19. The method of any one of embodiments D1-D18 wherein the polynucleotides of the oligonucleotide probes in the composition are capable of hybridizing to target nucleic acid fragments having an average GC content of about 40% to about 45%.

D20. The method of embodiment D19, wherein the average GC content is about 43%.

D20.1. The method of any one of embodiments D1-D20, comprising removing from a first set of designed external region directed oligonucleotide probes (i) oligonucleotide probes having a GC content less than about 40% and greater than about 60%, and (ii) oligonucleotide probes complementary to a subsequence of a nucleic acid that repeats in the nucleic acid fragments, thereby generating a second set of designed external region directed oligonucleotide probes.

D21. The method of any one of embodiments D1-D20, wherein the polynucleotides of the oligonucleotide probes in the composition are capable of hybridizing to target nucleic acid fragments having a GC content of about 1% to about 97%.

D22. The method of any one of embodiments D1-D21, wherein the oligonucleotide probes in the plurality of oligonucleotide probes comprise a biotin molecule.

D23. The method of any one of embodiments D1-D22, wherein the polynucleotide of each of the oligonucleotide probes comprises RNA.

D24. The method of any one of embodiments D1-D22, wherein the polynucleotide of each of the oligonucleotide probes consists of RNA.

D25. The method of any one of embodiments D1-D24, wherein the target nucleic acid fragments result from nucleic acid restriction enzyme cleavage by restriction enzymes cutting at \hat{ }GDTC and G\hat{ }DNTC, where “\hat{ }” represents the cut site on the positive DNA strand.

D26. The method of embodiment D25, wherein the restriction enzyme cleavage is by two restriction enzymes.

D27. The method of any one of embodiments D1-D26, wherein the plurality of oligonucleotide probes capable of hybridizing to target nucleic acid fragments of external regions and/or introns are not capable of hybridizing to tiled subsequences of a target nucleic acid fragment.

D28. The method of any one of embodiments D1-D27, wherein the plurality of oligonucleotide probes capable of hybridizing to target nucleic acid fragments of external regions and/or introns does not contain a probe capable of hybridizing to an end of a first target nucleic acid fragment and to an end of a second target nucleic acid fragment.

D29. The method of any one of embodiments D1-D28, wherein the first oligonucleotide probe and the second oligonucleotide probe in each of the oligonucleotide probe pairs do not hybridize to regions of a target nucleic acid fragment that overlap.

D30. The method of any one of embodiments D1-D29, wherein the plurality of oligonucleotide probes capable of hybridizing to target nucleic acid fragments of external regions and/or introns does not contain a probe capable of hybridizing to target nucleic acid fragment having a length of less than 130 consecutive nucleotides.

D31. The method of any one of embodiments D1-D30, wherein the oligonucleotide probes of the plurality of oligonucleotide probes capable of hybridizing to target nucleic acid fragments of external regions and/or introns capable of hybridizing to a target nucleic acid fragment are not capable of hybridizing to contiguous, non-overlapping regions of the nucleic acid fragment.

D32. The method of any one of embodiments D1-D31, wherein each oligonucleotide probe of a plurality of oligonucleotide probes capable of hybridizing to target nucleic acid fragments of exons and promoters is capable of hybridizing to a region spanning about 100 to about 500 consecutive nucleotides from the 5′ end of each target nucleic acid fragment or to a region spanning about 100 to about 500 consecutive nucleotides from the 3′ end of each target nucleic acid fragment.

D33. The method of any one of embodiments D1-D32, wherein the oligonucleotide probes of the plurality of oligonucleotide probes capable of hybridizing to target nucleic acid fragments of exons and promoters are capable of hybridizing to a region at a first region spanning about 350 consecutive nucleotides from the 5′ end of each target nucleic acid fragment or to a region spanning about 350 consecutive nucleotides from the 3′ end of each target nucleic acid fragment.

D34. The method of any one of embodiments D1-D33, wherein the oligonucleotide probes of the plurality of oligonucleotide probes capable of hybridizing to target nucleic acid fragments of exons and promoters capable of hybridizing to a target nucleic acid fragment are capable of hybridizing to contiguous, non-overlapping regions of the nucleic acid fragment.

D35. The method of any one of embodiments D1-D34, wherein the oligonucleotide probes of the plurality of oligonucleotide probes capable of hybridizing to target nucleic acid fragments of exons and promoters are not restricted to a GC content percentage range or threshold.

D36. The method of any one of embodiments D1-D35, comprising about 20,000 or more oligonucleotide probes.

D37. The method of embodiment D36, comprising about 21,000 or more oligonucleotide probes.

D38. The method of any one of embodiments D1-D37, comprising about 15,000 to about 25,000 oligonucleotide probes.

D39. The method of any one of embodiments D1-D37, comprising about 18,000 to about 24,000 oligonucleotide probes.

D40. The method of any one of embodiments D1-D37, comprising about 19,000 to about 23,000 oligonucleotide probes.

D41. The method of any one of embodiments D1-D37, comprising about 20,000 to about 22,000 oligonucleotide probes.

D42. The method of any one of embodiments D1-D41, comprising about 4,500 to about 8,000 unique oligonucleotide probes.

D43. The method of any one of embodiments D1-D41, comprising about 5,000 to about 7,500 unique oligonucleotide probes.

D44. The method of any one of embodiments D1-D41, comprising about 5,500 to about 7,000 unique oligonucleotide probes.

D45. The method of any one of embodiments D1-D41, comprising about 6,600 unique oligonucleotide probes.

D46. The composition of any one of embodiments D1-D45, wherein the external region comprises padding selected from the group of: 0 kb, + and/or − approximately 50 kb, + and/or − approximately 100 kb, + and/or − approximately 250 kb, and + and/or minus approximately 500 kb, + and/or minus approximately 1000 kb, + and/or minus approximately 2000 kb, + and/or minus approximately 5000 kb.

D47. The composition of any one of embodiments D1-D46, wherein a target cancer gene less than approximately 100 kb has extension regions added before padding is applied.

D48. The composition of any one of embodiments D1-D47, wherein if an upstream target gene upstream and a downstream target gene are adjacent or in close proximity in linear sequence on a chromosome, padding will only be applied upstream of the upstream target gene's promoter and downstream of the downstream gene's downstream most stop site.

E1. A method for preparing a composition comprising a plurality of oligonucleotide probes, the method comprising synthesizing oligonucleotide probes designed by the method of any one of embodiments D1-D48.

E2. The method of embodiment E1, comprising synthesizing the second set of designed external region directed oligonucleotide probes according to embodiment D20.1.

F1. A method for detecting the presence or absence of a structural variant in a sample, the method comprising analyzing sample nucleic acid from a subject, wherein the analyzing comprises:
generating proximity ligated nucleic acid molecules,
contacting the proximity ligated nucleic acid molecules with one or more oligonucleotide probes of any one of embodiments A1-A48, or designed by a method of any one of embodiments D1-D48, or prepared by a method of embodiment E1 or E2, thereby generating enriched proximity ligated nucleic acid molecules,
sequencing the enriched proximity ligated nucleic acid molecules, thereby generating sequences of the sample nucleic acid, and
determining the presence or absence of a structural variant in the sample nucleic acid from the sequences.

F2. The method of embodiment F1, comprising comparing sequences of the sample nucleic acid to sequences of a reference genome to determine the presence or absence of a structural variant.

### EXAMPLES

The examples set forth below illustrates certain implementations and does not limit the technology.

A panel of oligonucleotide probes was prepared for use in capturing a subset of target nucleic acids in proximally-ligated HiC libraries generated from biological sample nucleic acid preparations. Such a panel of capture oligonucleotide probes reduces the cost of sequencing per sample as compared to genome-wide sequence of nucleic acid prepared from a sample. The long-range information encoded in the proximally-ligated HiC libraries, which is the material enriched by the panel of oligonucleotide probes, enables detection of structural variants (SVs) outside of a gene body, referred to as neighborhood or proximal or proximity fusions. The oligonucleotide probes in the panel are RNA probes useful for capturing DNA target nucleic acid in proximally-ligated HiC libraries generated from samples (e.g., archived FFPE tissues), which are more stable than RNA in the samples.

Oligonucleotide probes that hybridize to 134 genes involved in tumors, presented in Appendix 2, were prepared and included in a comprehensive panel of oligonucleotide probes useful for capturing library nucleic acid and for identifying a wide variety of structural variants (SVs). Capture probes were designed to the exons, promoters, introns, and, in certain embodiments, external regions of the cancer genes. Promoters were defined as 1500 bp upstream to 500 bp downstream of the upstream most transcription start site of the gene. Each of the probes was 120 base pairs (bp) long and is designed to have a full 120 bp of complementarity to a fragment of the minus strand of the reference Genome assembly, GRCH38 (also referred to as HG38, hg38, and GRCh38). The average fragment size of the in silico digested fragments was 191 bp and the average % GC was 43.0% and ranged from 1.53% to 96.6%. There were approximately 22,000 individual probe oligonucleotide polynucleotides designed for the panel, comprising approximately 6, 600 unique sequences.

Target sequences were defined using scripts to identify regions around cut sites. The target regions for the exon and promoter probes were input into the AGILENT SUREDESIGN™ Software. All probes were manufactured as biotinylated RNA molecules. For exons and promoters, probes were generated via tiling at a 1× tiling density for 350 bp around each HiC restriction cut site that was contained in the exon and promoter sequences for the genes. The polynucleotide sequence of each of the probes was determined by in silico genome digestion using restriction enzymes cutting at \hat{ }GATC and G\hat{ }ANTC, where “\hat{ }” represents the cut site on the positive DNA strand and then using only the 350 bp of sequence around the in silico digested cut sites. Probe sequences designed to repetitive sequences in the genome were filtered out and removed from the panel of oligonucleotide probes using the “moderate Stringency” feature in the AGILENT

SUREDESIGN™ Software. For these probes, no restriction of the % GC content was made. Performance of the probes was normalized using “Boosting” feature in AGILENT SUREDESIGN™ Software, in which certain probes were replicated in the design to increase their relative abundance in the probe pool.

For introns and external regions, probes were designed using a sparse design that generates probes that are exactly 5 bp away from the restriction cut site (i.e., 5 bp away from the cleavage site on the positive strand, i.e., the first 5 double-stranded bases after digestion) using the same in silico digestion above but with some differences in how the probes were placed. Probes were not designed for restriction fragments that are less than 130 bp. A single probe was designed to the 5′ end cut site in restriction fragments that are between 130 bp and 259 bp in size. Two probes were designed to restriction fragments greater than or equal to 260 bp. Probes were removed if they have less than 40% GC and higher than 60% GC. Probes were removed if they are designed to repetitive regions in the genome. The “Boosting” feature in AGILENT SUREDESIGN™ Software was not utilized for these probes. The intron and external region probe sequences and replication rate in the probe pool (1 for all probes) was defined without using AGILENT SUREDESIGN™ Software. AGILENT SUREDESIGN™ Software was utilized only for purchasing intron and external region probes and not for designing the probes. The spacing of the intron and external region probes when hybridized to target nucleic acid fragments was significantly less dense than the spacing of hybridized exon and promoter probes. The spacing of hybridized intron external region probes was sparser than the spacing of hybridized exon and promoter probes due to the size restrictions of the fragments to which the probes are designed. This resulted in many fragments in introns not having probes designed to them or only a few having two probes designed to them.

The probes were designed with higher density tiling in the exons (i.e., 1× tiling) to allow for high resolution capture of structural variant (SV) break points involving the exons.

Oligonucleotide probes that hybridize to promoters were included in the panel. One reason to target promoters is to capture looping interactions with each of the genes, which may occur as a result of nearby SV's, referred to as “neoloops”. Oligonucleotide probes with sparser coverage of introns or external regions were included to provide resolution of SV's that occur in a gene body but outside of exon sequences. Sparser coverage of introns or external regions, as opposed to the full 1× tiling for promoters and exons, was included in the oligonucleotide probe design in part to reduce the overall cost of the oligonucleotide probe panel and in part to improve the overall performance and quality of the sequencing data obtained when using the panel.

Probe oligonucleotide performance was assessed in silico. In vivo genome-wide HiC data of a chordoma tumor was re-analyzed using “virtual” capture probes designed in accordance with various embodiments of the invention.

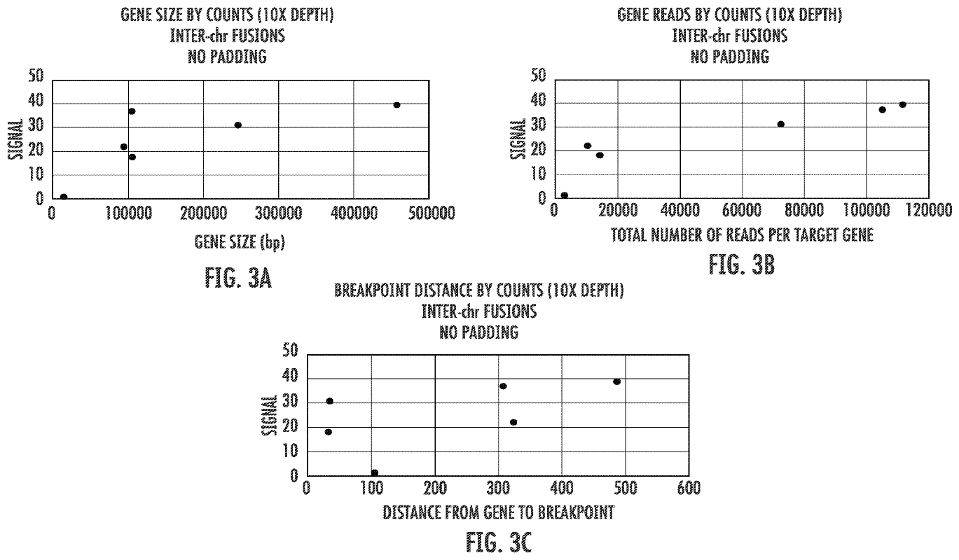

FIG. 3 shows that target gene size has a larger impact on signal strength, and thereby ability to enhance detection of structural variants, than breakpoint distance. Because of this finding, one embodiment of the invention enhances small target genes, under 100 kb as one non-limiting example, by extending the gene sequence included in the probe design equally and bilaterally both upstream from the upstream most transcription start site and downstream from the downstream most transcription stop site until the total length of the extended gene sequence was approximately 100 kb. The probes capable of hybridizing to the extension regions were designed the same way as intron and external region directed probes. With the smaller target genes, the extension regions are not considered part of the external region for padding (see below) purposes.

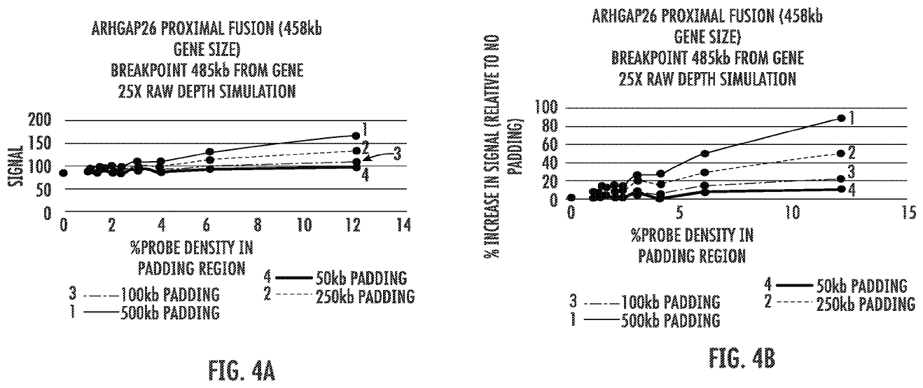

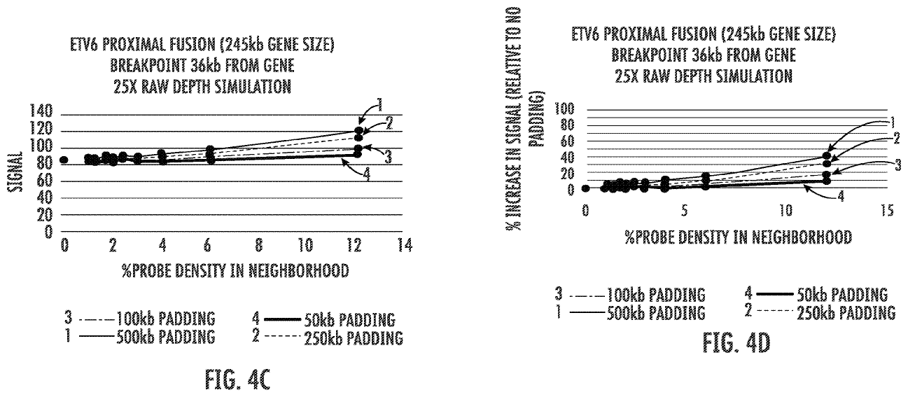

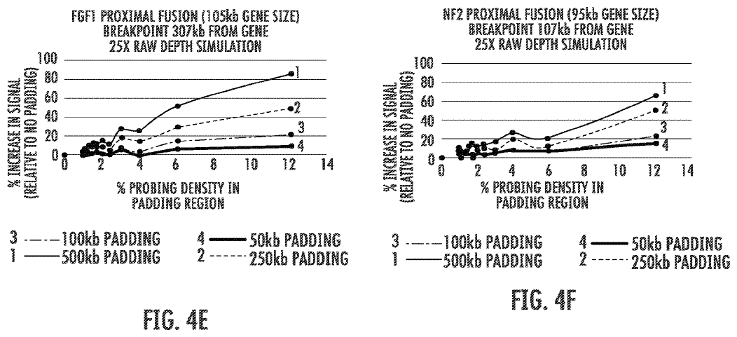

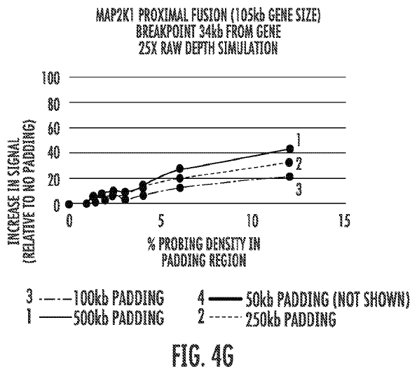

FIG. 4 shows various examples of how adding probes capable of hybridizing to external regions can increase the signal strength, thereby allowing easier and earlier detection of structural variants. In order to optimize signal strength, various probe densities (1 probe/1 kb (12%), 1 probe/2 kb, 1 probe/3 kb, 1 probe/4 kb, 1 probe/5 kb, 1 probe/6 kb, 1 probe/7 kb, 1 probe/8 kb, 1 probe/9 kb, 1 probe/10 kb, 1 probe/11 kb, 1 probe/12 kb (1%)) were tested with various amounts of padding. Padding is defined the amount of external region to which probes were designed. Padding, except in certain unique cases described below, was extended equally and bilaterally both upstream from the upstream end of a promoter region and downstream from the downstream most transcription stop site, so that 50 kb padding, for one-non-limiting example, means 50 kb upstream from the upstream end of a promoter region and 50 kb downstream from the downstream most transcription stop site were included in the probe design and those two 50 kb regions were considered external regions as used and described herein. Padding amounts tested were exons, introns, and promoters only (0 padding), +/−50 kb, +/−100 kb, +/−250 kb, and +/−500 kb.

In certain cases, a target cancer gene may be close to or adjacent to another target gene. In that case, padding was applied to be equal but unidirectional, so that padding would be applied upstream of the upstream end of the promoter region of the upstream target gene and downstream of the transcription stop site of the downstream target gene (in effect conjoining the two target genes for the purposes of probe design). FIGS. 4A and 4B show detection of ARHGAP26 proximal fusion. ARHGAP26 has a 486 kb gene size and the known breakpoint is 485 kb away from the nearest end of the gene. At 25× raw sequencing depth, the figures show that higher padding and high probe density increased proximity fusion signal strength (FIG. 4A). The largest amount of padding (500 kb) showed the highest percentage of increase in signal strength (FIG. 4B). Similarly, increase in signal (FIG. 4C) and percent increase in signal (FIG. 4D) were shown for a proximal fusion involving the ETV6 gene, a 245 kB gene with the fusion having a breakpoint 36 kb form the gene. Notably, by comparing the data from ARHGAP26 and ETV6, the % increase in signal strength is less when the breakpoint is closer to the gene. FIG. 4E shows similar results for percent signal increase for FGF1 proximal fusion, where FGFF1 is a 105 kb gene size and the fusion has a breakpoint 307 kb from the gene. FIG. 4F shows lesser percent signal increase than FGF proximal fusion, where NF2 is a 95 kb gene size and the fusion has a breakpoint 107 kb from the gene. In this example, even though NF2 is smaller than 100 kb, no extension was added. Lastly, FIG. 4G shows even lesser proximity fusion signal for MAP2K1 proximal fusion, where MAP2K1 has a gene size of 105 kb and the breakpoint is 34 kb from gene. Together FIGS. 4E-G have similarly sized genes, but decreasing distance to the breakpoints and correspondingly decreased proximity fusion signal, indicating that when gene size is constant, the effect of padding is reduced when the breakpoints are closer to the target gene. In all cases throughout FIG. 4, proximity fusion signal and percent increase of signal over no padding increased as the padding density and distance increased. Thus, FIG. 4 shows that including probes capable of hybridizing to external regions in a proximal ligation assay can improve detection of structural variants. The cancer genes used in the simulation are detailed below in TABLE 1. Within table 1, marker refers to the target gene in the virtual Capture-HiC analysis, “sample” is the sample name of the tumor used in the virtual Capture-HiC analysis, “type” refers to the type of genomic rearrangement, in which case all are inter-chromosomal (referred to as “inter”) translocations where the target gene and the genomic rearrangement partner region are located on different chromosomes. “gene size” refers to the length (in kilobases) of the target gene, and distance to breakpoint refers to the genomic distance between the target gene and the genome rearrangement breakpoint near the target gene.

TABLE 1

| | | | Gene size | Distance to |
|---|---|---|---|---|
| Marker | Sample | Type | (kb) | breakpoint (kb) |
| ETV6 | CF466 | inter | 245.5 | 36.4 |
| ARHGAP26 | CF466 | inter | 458.6 | 485.4 |
| FGF1 | CF466 | inter | 105.9 | 307.2 |
| NUTM1 | CF466 | inter | 14.4 | 107.3 |
| NF2 | CF466 | inter | 95.0 | 323.9 |
| MAP2K1 | CF466 | inter | 105.5 | 34.8 |

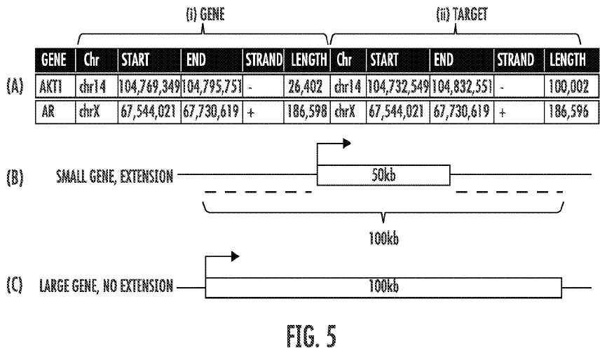

FIG. 5, panel A shows a table that shows how a small (defined as <100 kb, in this case the gene “AKT1”) has probes targeting the flanking regions (also known as extension regions) around (upstream and downstream) of the gene to bring the genomic length of their targeting (defined as the linear distance between the most upstream probe to most downstream probe associated with the targeting of that gene) to approximately 100 kb. More specifically, the gene itself has a length of approximately 26 Kb (with coordinates listed under “(i) Genes”), but after the design of probes targeting the flanking regions around (upstream and downstream) the gene, the genomic length of their targeting is approximately 100 kb (with coordinates listed under “(ii) Target”). In contrast, a large gene (defined as >100 kb, in this case the gene “AR”) has no difference between the gene coordinates and genomic length of targeting. Genomic coordinates are listed in hg38 human genome reference, and the gene annotation file used was genome-build-accession GCA_000001405.28, downloaded from here: https://www.ncbi.nlm.nih.gov/datasets/genome/GCF_000001405.39/. FIG. 5, panel B is a schematic example of the baseline probe design strategy for small genes (meaning how small genes are always targeted for both Group 1 and Group 2 genes), illustrating probes targeting the flanking regions (also known as extension regions) around (upstream and downstream) the gene to bring the genomic length of their targeting to approximately 100 kb. FIG. 5, panel C is a schematic example of the baseline probe design strategy for large genes, illustrating no probes targeting the flanking regions around (upstream and downstream) the gene. For both FIGS. 5B and 5C, the arrow indicates the start of the promoter, such that the figure is intended to illustrate only the additional probes targeting the flanking regions (in this case the extension regions) around (upstream and downstream) the gene.

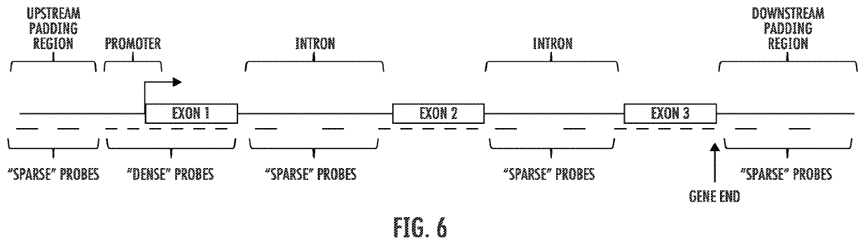

FIG. 6 shows a schematic of an embodiment of a probe design strategy for a large (>100 kb) Group 1 gene. Depicted here highlights the various features of a gene and how probes target the gene. Shown are exons, introns, promoter, and upstream and downstream regions of a large (>100 kb) Group 1 gene. Also indicated is whether probes are designed with a “dense” method, or a “sparse” method for each of the gene regions (i.e. exons, introns, promoter, and upstream and downstream regions). For example, promoters and exons are targeted with a “dense” method, which in this case means targeting the genomic sequences with probes with 1× probe tiling (each base is covered by 1 probe). As illustrated, in this case, since exons may not be divisible by the probe length, some exon-targeting probes overhang into the intronic sequence. In contrast, shown here is “sparse” method probe targeting of u m and downstream regions). For example, promoters and exons are targeted with a “dense” method, which in this case means targeting the genomic sequences with probes with 1× probe tiling (each base is covered by 1 probe). As illustrated, in this case, since exons may not be divisible by the probe length, some exon-targeting probes overhang into the intronic sequence. In contrast, shown here is “sparse” method probe targeting of upstream and downstream padding regions, as well as introns. The sparse method aims design probes such that 15% of bases in the targeted region are covered by 1 probe, and the specific probe placement is optimized for factors shown to improve probe performance such as considered the length of the restriction fragment for the targeted bases as well as their GC content.

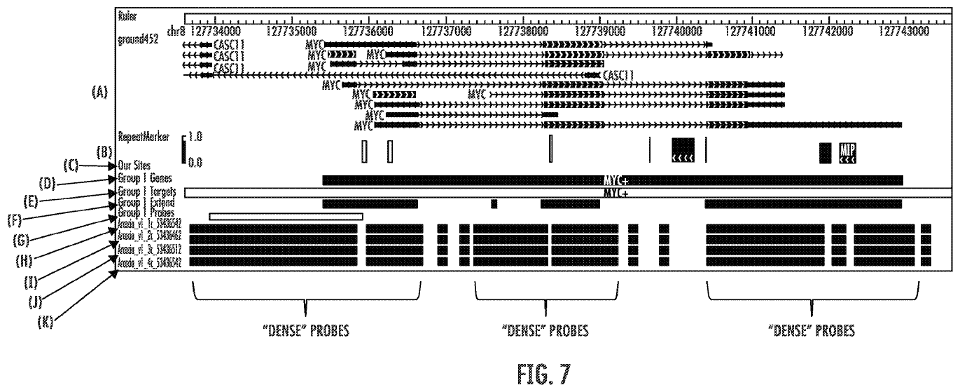

FIG. 7 shows a schematic of one example of probe placement in accordance with an embodiment of the invention around a Group 1 gene (MYC), focusing on probe design around promoter and exons. Row (A) shows Gencode v29 gene annotations for MYC (various different annotations shown in purple towards the center). Row (B) shows the location of repetitive sequences as identified by RepeatMasker. Row (C) shows the restriction enzyme cut site locations, for the restriction enzymes DpnII and HinfI used in the Capture-HiC sample preparation protocol. Row (D) shows the coordinates of the MYC gene, that was used as the basis for designing probes. Row (E) shows the genomic length of the probe targeting (defined as the linear span between the most upstream probe to most downstream probe associated with the targeting of that gene). In this case and in this figure, since MYC is a small gene (<100 kb), any targeting shown that is upstream of the promoter or downstream of the gene end is probe targeting associated with increasing the length of targeting to 100 kb (including the extension regions). Row (F) shows the coordinates of the MYC gene exons, that was used as the basis for designing probes to target exons. Row (G) shows the coordinates of the MYC gene promoter, that was used as the basis for designing probes to target the promoter. Row (H) shows the regions actually targeted by probes in a probe design set that has 0 kb of additional up/downstream padding beyond the gene (or beyond the 100 kb region for small genes). Row (I) shows the regions actually targeted by probes in a probe design set that has 100 kb of additional up/downstream padding beyond the gene (or beyond the 100 kb region for small genes). Row (J) shows the regions actually targeted by probes in a probe design set that has 250 kb of additional up/downstream padding beyond the gene (or beyond the 100 kb region for small genes). Row (K) shows the regions actually targeted by probes in a probe design set that has 500 kb of additional up/downstream padding beyond the gene (or beyond the 100 kb region for small genes). For Rows I, J, and K, the padding probe coverage is not visible in this view since it is zoomed in to the gene. Also note for Rows H, I, J, and K, what's shown is the regions actually targeted by probe. More specifically, this shows the output of the Agilent SUREDESIGN™ software probe placement according to our input target regions for promoters, exons, introns, extension regions for small genes, and additional padding regions. SUREDESIGN™ may mask repetitive sequences in probe design, which can be seen here as small gaps in probe coverage (in rows H, I, J, and K) within each exon. Further highlighted here are the regions of “Dense” probes, which are allocated to promoters and exons. All data here have been visualized using the WashU Genome Browser, in a genomic window spanning approximately chr8: 127, 733, 500-127, 743, 500.

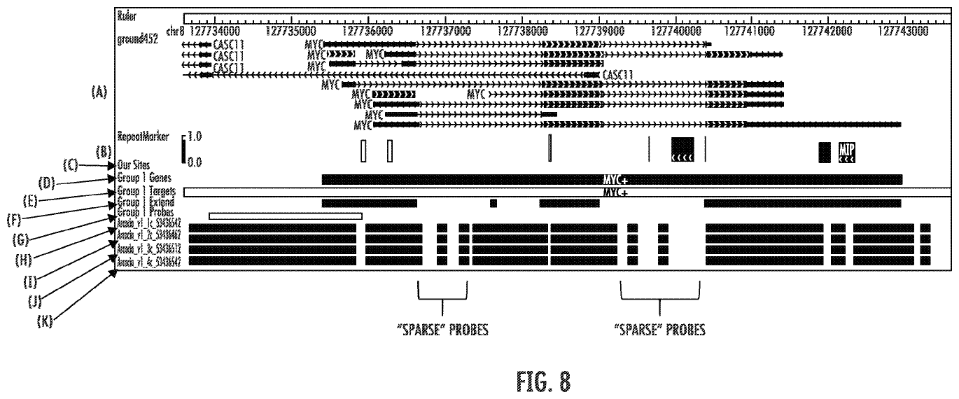

FIG. 8 shows a schematic of an example of one embodiment of probe placement around a Group 1 gene (MYC), focusing on probe design around introns. Row (A) shows Gencode v29 gene annotations for MYC (various different annotations shown in purple towards the center). Row (B) shows the location of repetitive sequences as identified by RepeatMasker. Row (C) shows the restriction enzyme cut site locations, for the restriction enzymes DpnII and HinfI used in the Capture-HiC sample preparation protocol. Row (D) shows the coordinates of the MYC gene, that was used as the basis for designing probes. Row (E) shows the genomic length of the probe targeting (defined as the linear span between the most upstream probe to most downstream probe associated with the targeting of that gene). In this case and in this figure, since MYC is a small gene (<100 kb), any targeting shown that is upstream of the promoter or downstream of the gene end is probe targeting associated with increasing the length of targeting to 100 kb (including the extension regions). Row (F) shows the coordinates of the MYC gene exons, that was used as the basis for designing probes to target exons. Row (G) shows the coordinates of the MYC gene promoter, that was used as the basis for designing probes to target the promoter. Row (H) shows the regions actually targeted by probes in a probe design set that has 0 kb of additional up/downstream padding beyond the gene (or beyond the 100 kb region for small genes). Row (I) shows the regions actually targeted by probes in a probe design set that has 100 kb of additional up/downstream padding beyond the gene (or beyond the 100 kb region for small genes). Row (J) shows the regions actually targeted by probes in a probe design set that has 250 kb of additional up/downstream padding beyond the gene (or beyond the 100 kb region for small genes). Row (K) shows the regions actually targeted by probes in a probe design set that has 500 kb of additional up/downstream padding beyond the gene (or beyond the 100 kb region for small genes). For Rows I, J, and K, the padding probe coverage is not visible in this view since it is zoomed in to the gene. Also note for Rows H, I, J, and K, what's shown is the regions actually targeted by probe. More specifically, this shows the output of the Agilent SUREDESIGN™ software probe placement according to our input target regions for promoters, exons, introns, extension regions for small genes, and additional padding regions. SUREDESIGN™ may mask repetitive sequences in probe design, which can be seen here as small gaps in probe coverage (in rows H, I, J, and K) within each exon. Further highlighted here is the regions of “Sparse” probes, which are allocated to introns. All data here have been visualized using the WashU Genome Browser, in a genomic window spanning approximately chr8: 127, 733, 500-127,743,500.

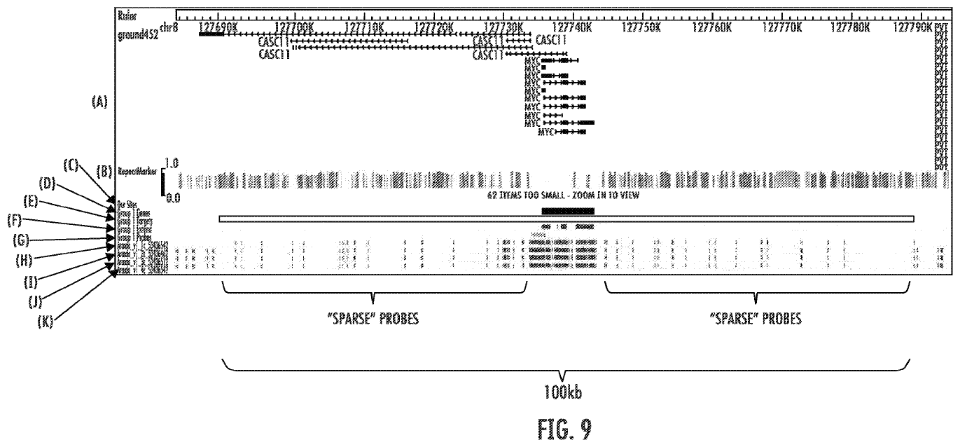

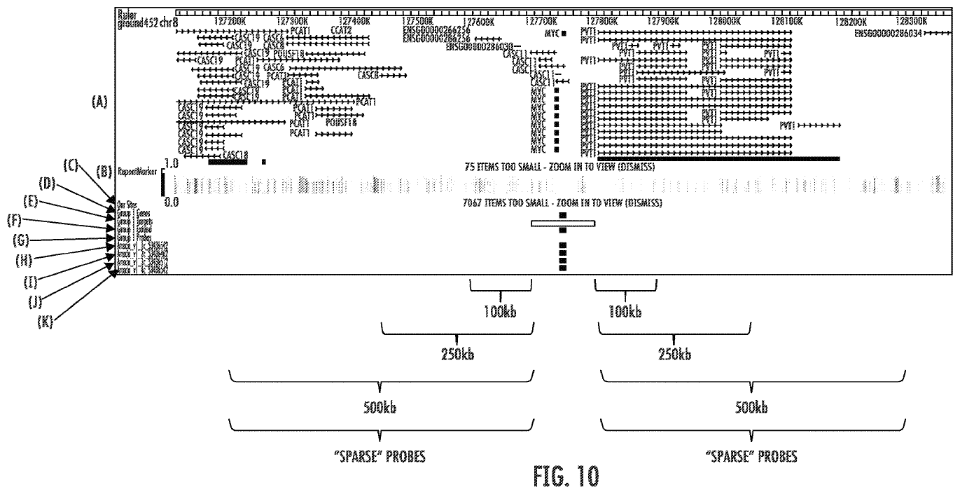

FIG. 9 shows a schematic of an example of one embodiment of probe placement around a Group 1 gene (MYC), focusing on probes targeting the upstream and downstream regions of MYC that are associated with increasing the length of targeting to 100 kb. Row (A) shows Gencode v29 gene annotations for MYC (various different annotations shown in purple towards the center). Row (B) shows the location of repetitive sequences as identified by RepeatMasker. Row (C) would show the restriction enzyme cut site locations, for the restriction enzymes DpnII and HinfI used in the Capture-HiC sample preparation protocol, but are not plotted due to low level of zoom for this genomic window. Row (D) shows the coordinates of the MYC gene, that was used as the basis for designing probes. Row (E) shows the genomic length of the probe targeting (defined as the linear span between the most upstream probe to most downstream probe associated with the targeting of that gene). In this case and in this figure, since MYC is a small gene (<100 kb), any targeting shown that is upstream of the promoter or downstream of the gene end is probe targeting associated with increasing the length of targeting to 100 kb. Row (F) shows the coordinates of the MYC gene exons, that was used as the basis for designing probes to target exons. Row (G) shows the coordinates of the MYC gene promoter, that was used as the basis for designing probes to target the promoter. Row (H) shows the regions actually targeted by probes in a probe design set that has 0 kb of additional up/downstream padding beyond the gene (or beyond the 100 kb region for small genes). Row (I) shows the regions actually targeted by probes in a probe design set that has 100 kb of additional up/downstream padding beyond the gene (or beyond the 100 kb region for small genes). Row (J) shows the regions actually targeted by probes in a probe design set that has 250 kb of additional up/downstream padding beyond the gene (or beyond the 100 kb region for small genes). Row (K) shows the regions actually targeted by probes in a probe design set that has 500 kb of additional up/downstream padding beyond the gene (or beyond the 100 kb region for small genes). Note for Rows H, I, J, and K, what's shown are the regions actually targeted by probe. More specifically, this shows the output of the Agilent SUREDESIGN™ software probe placement according to our input target regions for promoters, exons, introns, extension regions for small genes, and additional padding regions. Highlighted here for Rows H, I, J, and K, are the probes placed beyond the gene in the upstream and downstream regions of MYC that are associated with increasing the length of targeting to 100 kb. These are the regions indicated with brackets and indicated as “Sparse” probes. Also of note, the entire region of MYC plus the probes placed beyond the gene in the upstream and downstream regions of MYC has a total length of 100 kb, as indicated by the bottom-most brackets and text. All data here have been visualized using the WashU Genome Browser, in a genomic window spanning approximately chr8: 127,684,000-127,794,000. FIG. 10 shows a schematic of one example of an embodiment of probe placement around a Group 1 gene (MYC), focusing on the additional probes in the up/downstream padding regions beyond the gene (or in this case beyond the targeted 100 kb region because MYC is a small gene). Row (A) shows Gencode v29 gene annotations for MYC (various different annotations shown in purple towards the center). Row (B) shows the location of repetitive sequences as identified by RepeatMasker. Row (C) would show the restriction enzyme cut site locations, for the restriction enzymes DpnII and HinfI used in the Capture-HiC sample preparation protocol, but are not plotted due to low level of zoom for this genomic window. Row (D) shows the coordinates of the MYC gene, that was used as the basis for designing probes. Row (E) shows the genomic length of the probe targeting (defined as the linear span between the most upstream probe to most downstream probe associated with the targeting of that gene). In this case and in this figure, since MYC is a small gene (<100 kb), any targeting shown that is upstream of the promoter or downstream of the gene end is probe targeting associated with increasing the length of targeting to 100 kb. Row (F) shows the coordinates of the MYC gene exons, that was used as the basis for designing probes to target exons. Row (G) shows the coordinates of the MYC gene promoter, that was used as the basis for designing probes to target the promoter. Row (H) shows the regions actually targeted by probes in a probe design set that has 0 kb of additional up/downstream padding beyond the gene (or beyond the 100 kb region for small genes). Row (I) shows the regions actually targeted by probes in a probe design set that has 100 kb of additional up/downstream padding beyond the gene (or beyond the 100 kb region for small genes). This additional 100 kb is indicated in brackets and labeled as “100 kb” below the browser snapshot. Row (J) shows the regions actually targeted by probes in a probe design set that has 250 kb of additional up/downstream padding beyond the gene (or beyond the 100 kb region for small genes). This additional 250 kb is indicated in brackets and labeled as “250 kb” below the browser snapshot. Row (K) shows the regions actually targeted by probes in a probe design set that has 500 kb of additional up/downstream padding beyond the gene (or beyond the 100 kb region for small genes). This additional 500 kb is indicated in brackets and labeled as “500 kb” below the browser snapshot. Note for Rows H, I, J, and K, what's shown is the regions actually targeted by probe. More specifically, this shows the output of the Agilent SUREDESIGN (TM) software probe placement according to our input target regions for promoters, exons, introns, extension regions for small genes, and additional padding regions. Highlighted here for Rows H, I, J, and K, are the probes in the additional padding regions beyond the gene (or in this case beyond the targeted 100 kb region because MYC is a small gene). These are the regions indicated with brackets and indicated as “Sparse” probes. All data here have been visualized using the WashU Genome Browser, in a genomic window spanning approximately chr8: 127,110,000-128,360,000.

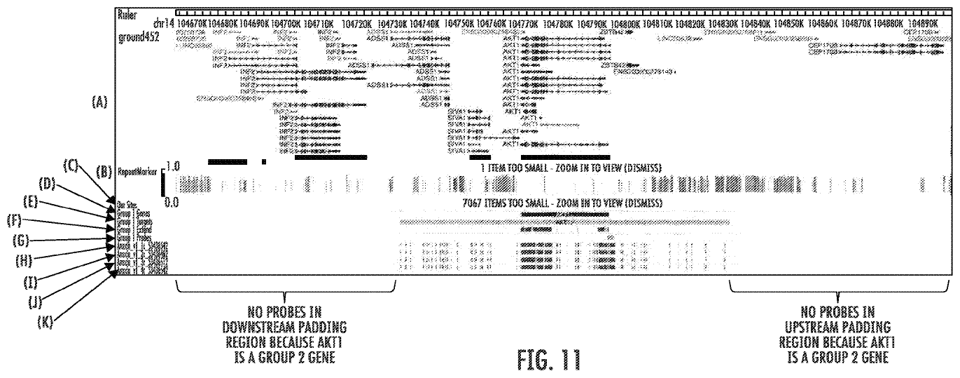

FIG. 11 shows a schematic of one example of an embodiment of probe placement around a Group 2 gene (AKT1), focusing on how there are no additional probes in the up/downstream padding regions beyond the gene (or in this case beyond the targeted 100 kb region because AKT1 is a small gene). Row (A) shows Gencode v29 gene annotations for AKT1 (various different annotations shown in purple and red towards the center). Row (B) shows the location of repetitive sequences as identified by RepeatMasker. Row (C) would show the restriction enzyme cut site locations, for the restriction enzymes DpnII and HinfI used in the Capture-HiC sample preparation protocol, but are not plotted due to low level of zoom for this genomic window. Row (D) shows the coordinates of the AKT1 gene, that was used as the basis for designing probes. Row (E) shows the genomic length of the probe targeting (defined as the linear span between the most upstream probe to most downstream probe associated with the targeting of that gene). In this case and in this figure, since AKT1 is a small gene (<100 kb), any targeting shown that is upstream of the promoter or downstream of the gene end is probe targeting associated with increasing the length of targeting to 100 kb. Row (F) shows the coordinates of the AKT1 gene exons, that was used as the basis for designing probes to target exons. Row (G) shows the coordinates of the AKT1 gene promoter, that was used as the basis for designing probes to target the promoter. Row (H) shows the regions actually targeted by probes in a probe design set that has 0 kb of additional up/downstream padding beyond the gene (or beyond the 100 kb region for small genes). Row (I) shows the regions actually targeted by probes in a probe design set that has 100 kb of additional up/downstream padding beyond the gene (or beyond the 100 kb region for small genes). Note that because AKT1 is a Group 2 gene, no additional probes are placed. Row (J) shows the regions actually targeted by probes in a probe design set that has 250 kb of additional up/downstream padding beyond the gene (or beyond the 100 kb region for small genes). Note that because AKT1 is a Group 2 gene, no additional probes are placed. Row (K) shows the regions actually targeted by probes in a probe design set that has 500 kb of additional up/downstream padding beyond the gene (or beyond the 100 kb region for small genes). Note that because AKT1 is a Group 2 gene, no additional probes are placed. Note for Rows H, I, J, and K, what's shown is the regions actually targeted by probe. More specifically, this shows the output of the Agilent SUREDESIGN™ software probe placement according to our input target regions for promoters, exons, introns, extension regions for small genes, and additional padding regions (for Group 1 genes, so not applicable in this example of AKT1). Highlighted here for Rows H, I, J, and

K, are the absence of probes in the additional padding regions beyond the gene (or in this case beyond the targeted 100 kb region because AKT1 is a small gene). All data here have been visualized using the WashU Genome Browser, in a genomic window spanning approximately chr14: 104,665,000-104,900,000.

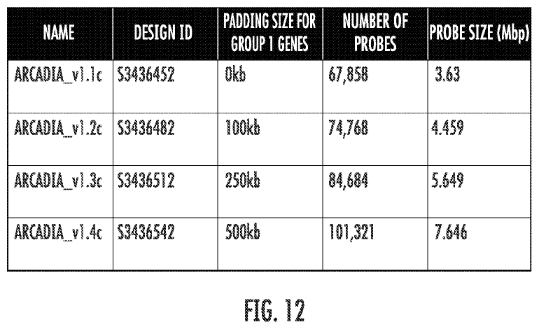

FIG. 12 shows a table of 4 different probe design sets in accordance with various embodiments of the invention. The key difference in their design strategy is the difference in the length of the additional probes in the up/downstream padding regions beyond the group 1 genes (or beyond the targeted 100 kb region for small Group 1 genes). This length is indicated in the column headed “Padding Size for Group 1 Genes”. To the right of that, the number of probes actually designed by Agilent SUREDESIGN™ software according to our input target regions for promoters, exons, introns, extension regions for small genes, and additional padding regions (for Group 1 genes). To the right of that is the Probe Size, which is the cumulate length (in megabase pairs, Mbp) of genomic bases covered by probe.

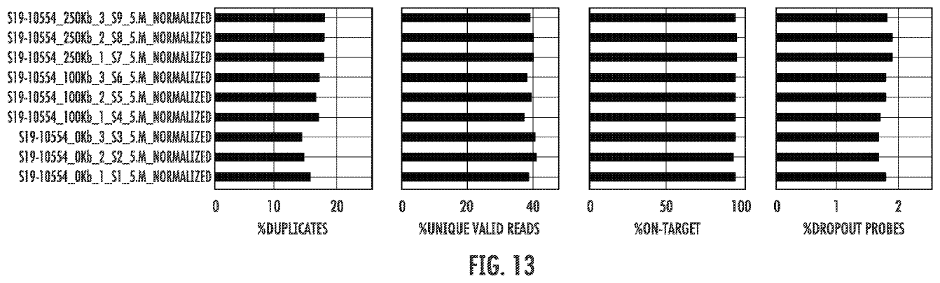

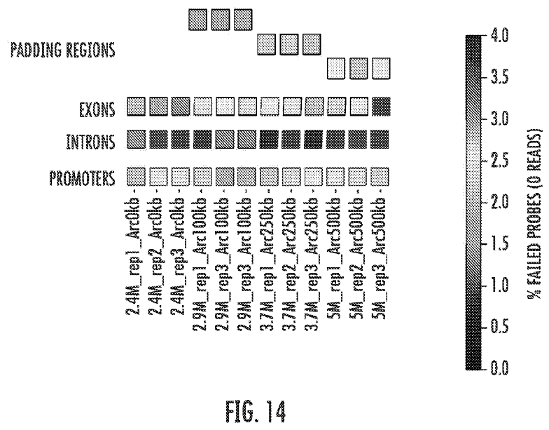

FIG. 13 shows graphical results of experiments where Arima Capture-HiC was performed using 4 different probe design sets (0 kb additional padding, 100 kb additional padding, 250 kb additional padding, and 500 kb additional padding) in accordance with embodiments of the invention, in triplicate for each probe design set, using an FFPE gynecological sarcoma tumor specimen. To analyze the data, the average sequencing fold-coverage over the regions actually targeted by probe given 5M total raw read-pairs was determined for the datasets from the 500 kb additional padding probe design set. The remaining datasets for 0 kb additional padding, 100 kb additional padding, 250 kb additional padding were then normalized to have the same average sequencing fold-coverage over the regions actually targeted by probe as the 500 kb additional padding datasets. This effectively normalizes all the datasets based on average sequencing fold-coverage over the regions actually targeted by probe, rather than raw reads, since the number of probes and probe size is different for each probe design set. We then calculated performance metrics in the data, including the % of PCR duplicates (left most bar plot), unique valid pairs (UVPs) as computed by the HiCUP data analysis pipeline, the % of reads on-target, and the % of dropout probes (which corresponds to the % of probes with 0 overlapping NGS reads). All probe design sets are observed to have quantitatively similar performance according to these metrics. The top 3 rows are the triplicates from the 250 kb additional padding datasets, the next 3 rows are the triplicates from the 100 kb additional padding datasets and the bottom 3 rows are the triplicates from the 0 kb additional padding datasets.

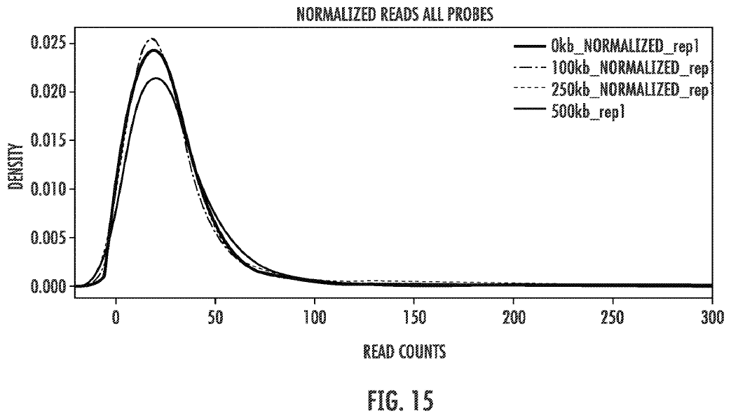

Figure shows graphical results of experiments where Arima Capture-HiC was performed using 4 different probe design sets (0 kb additional padding, 100 kb additional padding, 250 kb additional padding, and 500 kb additional padding) in accordance with various embodiments of the invention, in triplicate for each probe design set, using an FFPE gynecological sarcoma tumor specimen. To analyze the data, the average sequencing fold-coverage over the regions actually targeted by probe given 5M total raw read-pairs was determined for the datasets from the 500 kb additional padding probe design set. The remaining datasets for 0 kb additional padding, 100 kb additional padding, 250 kb additional padding were then normalized to have the same average sequencing fold-coverage over the regions actually targeted by probe as the 500 kb additional padding datasets. This effectively normalizes all the datasets based on average sequencing fold-coverage over the regions actually targeted by probe, rather than raw reads, since the number of probes and probe size is different for each probe design set. We then calculated the % of failed probes (which corresponds to the % of probes with 0 overlapping NGS reads), for the various different target types within and external to the target genes (e.g. the padding regions, exons, introns, and promoters. The left 3 columns are datasets from the 0 kb additional padding, the next 3 columns are datasets from the 100 kb additional padding, the next 3 columns are datasets from the 250 kb additional padding, and the last 3 (right most) columns are datasets from the 500 kb additional padding. As expected, promoters and exons having relatively higher % of failed probes (~2-4%) than introns (~1%), likely because probes in introns were designed with optimized restriction fragment length and GC content considerations, whereas probes in exons and promoters were optimized for sequence coverage even if sub-optimal restriction fragment length and GC content parameters. The four different probe design sets have quantitatively similar probe failure profiles. FIG. 15 shows graphical results of experiments where Arima Capture-HiC was performed using 4 different probe design sets (0 kb additional padding, 100 kb additional padding, 250 kb additional padding, and 500 kb additional padding) in accordance with various embodiments of the invention, in triplicate for each probe design set, using an FFPE gynecological sarcoma tumor specimen. To analyze the data, the average sequencing fold-coverage over the regions actually targeted by probe given 5M total raw read-pairs was determined for the datasets from the 500 kb additional padding probe design set. The remaining datasets for 0 kb additional padding, 100 kb additional padding, 250 kb additional padding were then normalized to have the same average sequencing fold-coverage over the regions actually targeted by probe as the 500 kb additional padding datasets. This effectively normalizes all the datasets based on average sequencing fold-coverage over the regions actually targeted by probe, rather than raw reads, since the number of probes and probe size is different for each probe design set. We then generated a coverage histogram over the regions actually targeted by probe for each of the 4 probe design sets. All 4 probe design sets have quantitatively similar coverage distributions, with most probes have coverage that fits a narrow distribution, and few probes fail.

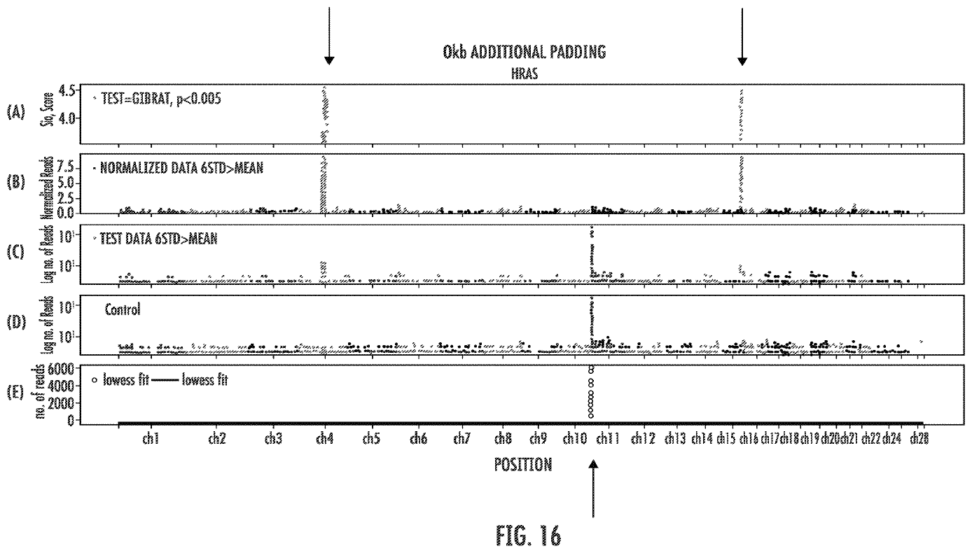

FIG. 16 shows Manhattan plots from experiment(s) where Arima Capture-HiC was performed using the 0 kb additional padding probe design set in accordance with an embodiment of the invention, using a cancer sample with a known breakpoint adjacent to the HRAS gene, and whereby the probe design set contains probes targeting the HRAS gene (a Group 1 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to HRAS.

Row A—Manhattan plot showing data from the cancer sample, showing the data from Panel B except transformed into a statistical significance score for the y-axis. Because the y-axis does not start at 0, only bins with a statistical significance score greater than approximately 3.5 are shown. This visual helps quickly visualize the statistically significant regions, which would be inferred to the genomic rearrangement partners with the target gene. Further, colored in purple are the significance scores at certain genomic bins whereby the significance scores in those bins is corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

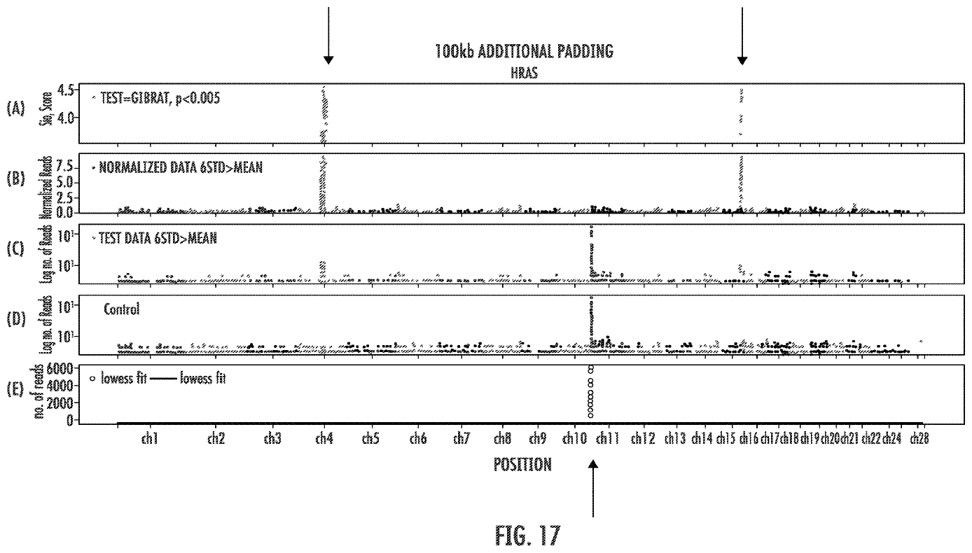

FIG. 17 shows Manhattan plots from experiment(s) where Arima Capture-HiC was performed using the 100 kb additional padding probe design set in accordance with an embodiment of the invention, using a cancer sample with a known breakpoint adjacent to the HRAS gene, and whereby the probe design set contains probes targeting the HRAS gene (a Group 1 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to HRAS. Row A—Manhattan plot showing data from the cancer sample, showing the data from Panel B except transformed into a statistical significance score for the y-axis. Because the y-axis does not start at 0, only bins with a statistical significance score greater than approximately 3.5 are shown. This visual helps quickly visualize the statistically significant regions, which would be inferred to the genomic rearrangement partners with the target gene. Further, colored in purple are the significance scores at certain genomic bins whereby the significance scores in those bins is corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

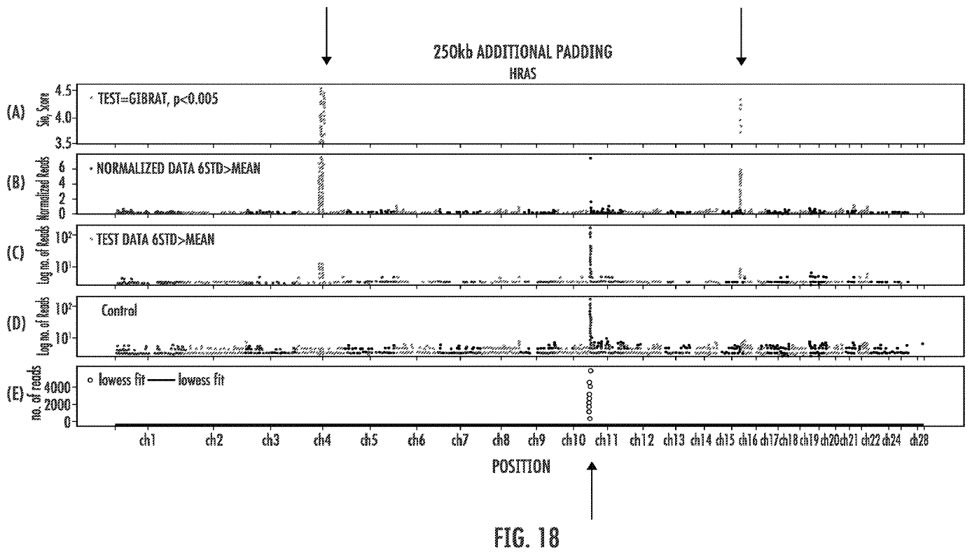

FIG. 18 shows Manhattan plots from experiment(s) where Arima Capture-HiC was performed using the 100 kb additional padding probe design set in accordance with an embodiment of the invention, using a cancer sample with a known breakpoint adjacent to the HRAS gene, and whereby the probe design set contains probes targeting the HRAS gene (a Group 1 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to HRAS.

Row A—Manhattan plot showing data from the cancer sample, showing the data from Panel B except transformed into a statistical significance score for the y-axis. Because the y-axis does not start at 0, only bins with a statistical significance score greater than approximately 3.5 are shown. This visual helps quickly visualize the statistically significant regions, which would be inferred to the genomic rearrangement partners with the target gene. Further, colored in purple are the significance scores at certain genomic bins whereby the significance scores in those bins corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

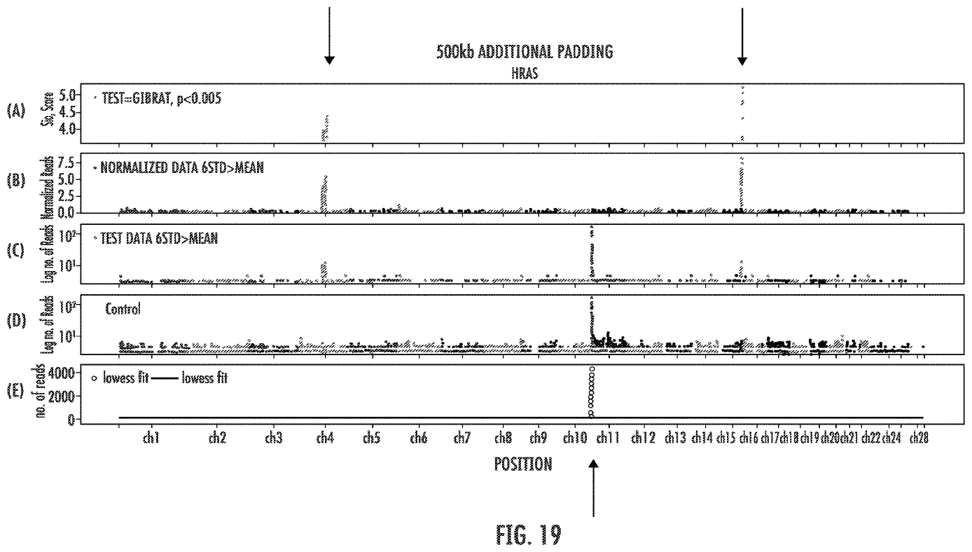

FIG. 19 shows Manhattan plots from experiment(s) where Arima Capture-HiC was performed using the 500 kb additional padding probe design set in accordance with an embodiment of the invention, using a cancer sample with a known breakpoint adjacent to the HRAS gene, and whereby the probe design set contains probes targeting the HRAS gene (a Group 1 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to HRAS.

Row A—Manhattan plot showing data from the cancer sample, showing the data from Panel B except transformed into a statistical significance score for the y-axis. Because the y-axis does not start at 0, only bins with a statistical significance score greater than approximately 3.5 are shown. This visual helps quickly visualize the statistically significant regions, which would be inferred to the genomic rearrangement partners with the target gene. Further, colored in purple are the significance scores at certain genomic bins whereby the significance scores in those bins is corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

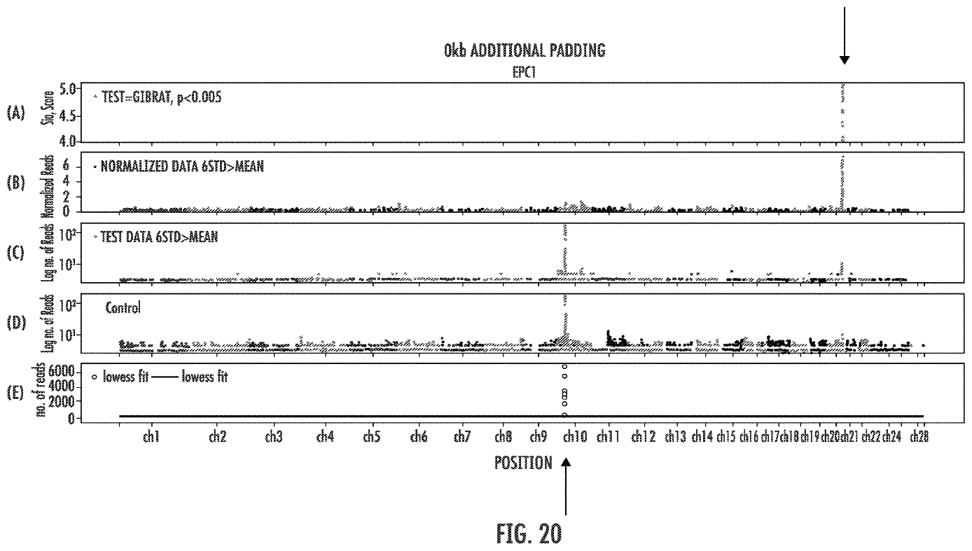

FIG. 20 shows Manhattan plots from experiment(s) where Arima Capture-HiC was performed using the 0 kb additional padding probe design set in accordance with an embodiment of the invention, using a cancer sample with a known breakpoint adjacent to the EPC1 gene, and whereby the probe design set contains probes targeting the EPC1 gene (a Group 2 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to EPC1.

Row A—Manhattan plot showing data from the cancer sample, showing the data from Panel B except transformed into a statistical significance score for the y-axis. Because the y-axis does not start at 0, only bins with a statistical significance score greater than approximately 4 are shown. This visual helps quickly visualize the statistically significant regions, which would be inferred to the genomic rearrangement partners with the target gene. Further, colored in purple are the significance scores at certain genomic bins whereby the significance scores in those bins is corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

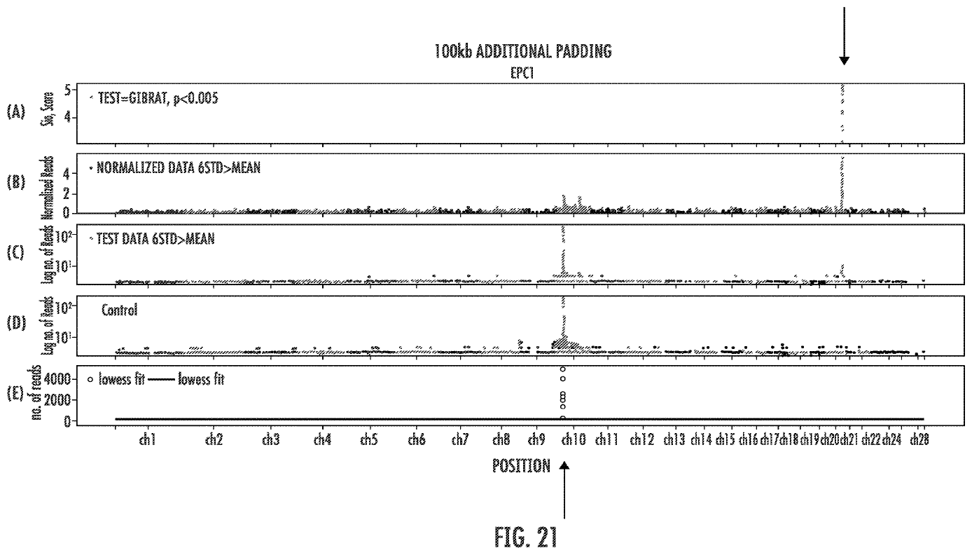

FIG. 21 shows Manhattan plots from experiment(s) where Arima Capture-HiC was performed using the 100 kb additional padding probe design set in accordance with an embodiment of the invention, using a cancer sample with a known breakpoint adjacent to the EPC1 gene, and whereby the probe design set contains probes targeting the EPC1 gene (a Group 2 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to EPC1.

Row A—Manhattan plot showing data from the cancer sample, showing the data from Panel B except transformed into a statistical significance score for the y-axis. Because the y-axis does not start at 0, only bins with a statistical significance score greater than approximately 3 are shown. This visual helps quickly visualize the statistically significant regions, which would be inferred to the genomic rearrangement partners with the target gene. Further, colored in purple are the significance scores at certain genomic bins whereby the significance scores in those bins is corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

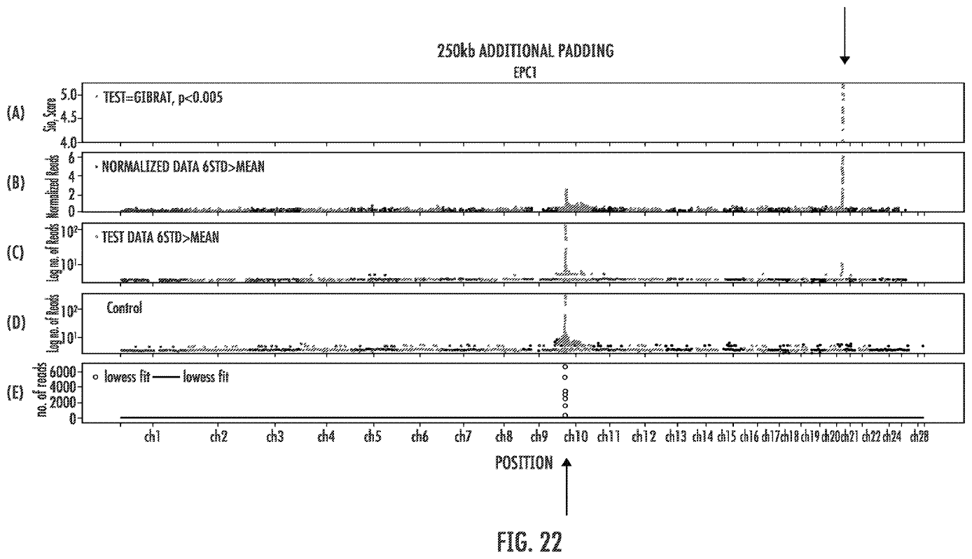

FIG. 22 shows Manhattan plots from experiment(s) where Arima Capture-HiC was performed using the 250 kb additional padding probe design set in accordance with an embodiment of the invention, using a cancer sample with a known breakpoint adjacent to the EPC1 gene, and whereby the probe design set contains probes targeting the EPC1 gene (a Group 2 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to EPC1.

Row A—Manhattan plot showing data from the cancer sample, showing the data from Panel B except transformed into a statistical significance score for the y-axis. Because the y-axis does not start at 0, only bins with a statistical significance score greater than approximately 4 are shown. This visual helps quickly visualize the statistically significant regions, which would be inferred to the genomic rearrangement partners with the target gene. Further, colored in purple are the significance scores at certain genomic bins whereby the significance scores in those bins is corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

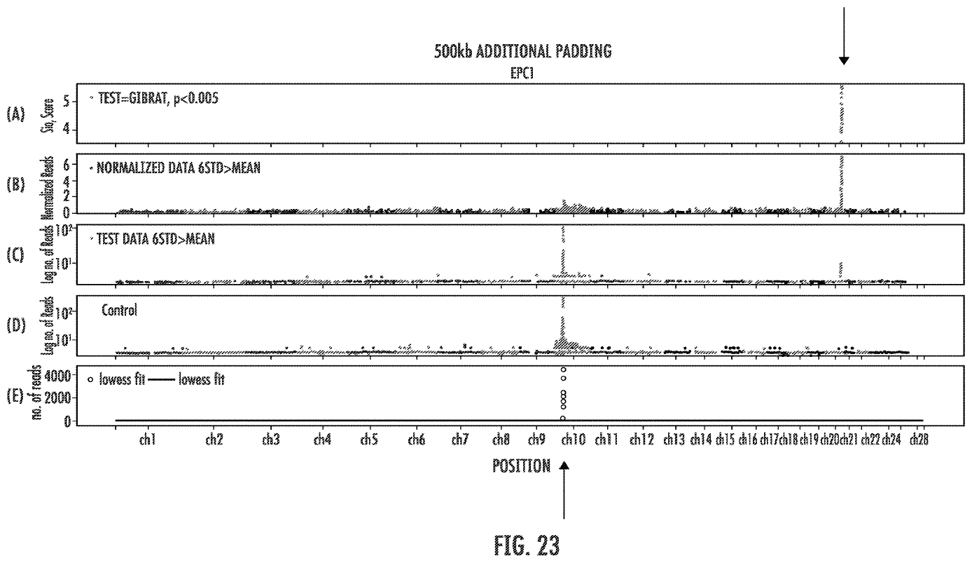

FIG. 23 shows Manhattan plots from experiment(s) where Arima Capture-HiC was performed using the 500 kb additional padding probe design set in accordance with an embodiment of the invention, using a cancer sample with a known breakpoint adjacent to the EPC1 gene, and whereby the probe design set contains probes targeting the EPC1 gene (a Group 2 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to EPC1.

Row A—Manhattan plot showing data from the cancer sample, showing the data from Panel B except transformed into a statistical significance score for the y-axis. Because the y-axis does not start at 0, only bins with a statistical significance score greater than approximately 3.5 are shown. This visual helps quickly visualize the statistically significant regions, which would be inferred to the genomic rearrangement partners with the target gene. Further, colored in purple are the significance scores at certain genomic bins whereby the significance scores in those bins is corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

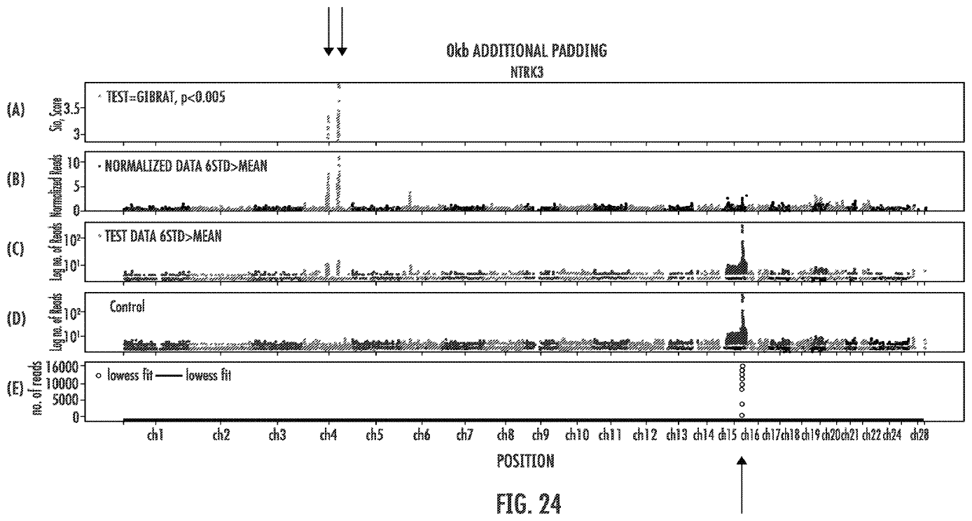

FIG. 24 shows Manhattan plots from experiment(s) where Arima Capture-HiC was performed using the 0 kb additional padding probe design set in accordance with an embodiment of the invention, using a cancer sample with a known breakpoint adjacent to the NTRK3 gene, and whereby the probe design set contains probes targeting the NTRK3 gene (a Group 1 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to NTRK3. Row A—Manhattan plot showing data from the cancer sample, showing the data from Panel B except transformed into a statistical significance score for the y-axis. Because the y-axis does not start at 0, only bins with a statistical significance score greater than approximately 3 are shown. This visual helps quickly visualize the statistically significant regions, which would be inferred to the genomic rearrangement partners with the target gene. Further, colored in purple are the significance scores at certain genomic bins whereby the significance scores in those bins is corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

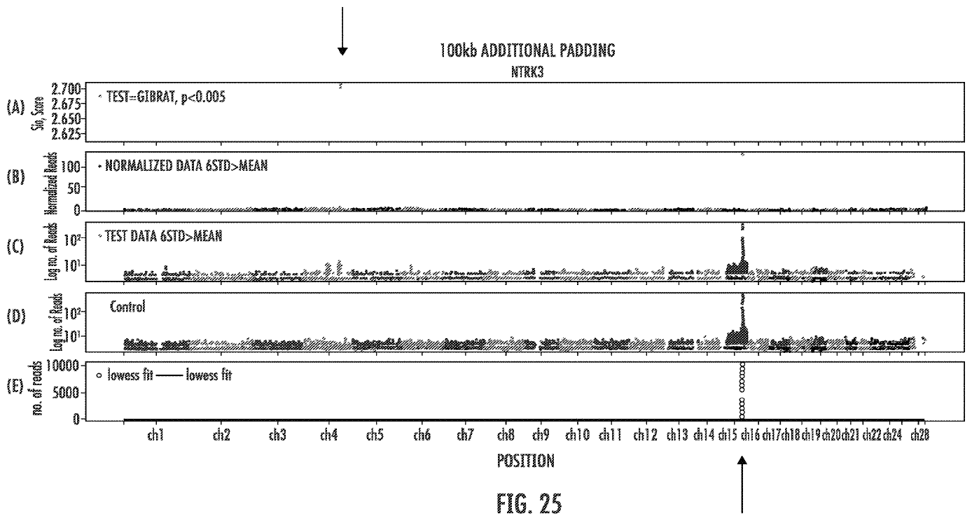

FIG. 25 shows Manhattan plots from experiment(s) where Arima Capture-HiC was performed using the 100 kb additional padding probe design set in accordance with an embodiment of the invention,
a cancer sample with a known breakpoint adjacent to the NTRK3 gene, and whereby the probe design set contains probes targeting the NTRK3 gene (a Group 1 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to NTRK3.

Row A—Manhattan plot showing data from the cancer sample, showing the data from Panel B except transformed into a statistical significance score for the y-axis. Because the y-axis does not start at 0, only bins with a statistical significance score greater than approximately 2.6 are shown. This visual helps quickly visualize the statistically significant regions, which would be inferred to the genomic rearrangement partners with the target gene. Further, colored in purple are the significance scores at certain genomic bins whereby the significance scores in those bins is corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

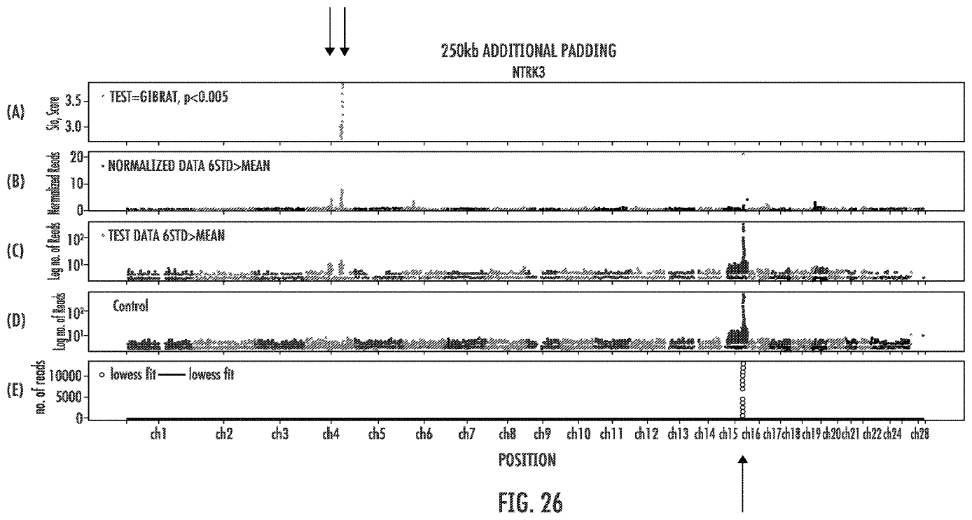

FIG. 26 shows Manhattan plots from experiment(s) where Arima Capture-HiC was performed using the 250 kb additional padding probe design set in accordance with an embodiment of the invention, using a cancer sample with a known breakpoint adjacent to the NTRK3 gene, and whereby the probe design set contains probes targeting the NTRK3 gene (a Group 1 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to NTRK3. Row A—Manhattan plot showing data from the cancer sample, showing the data from Panel B except transformed into a statistical significance score for the y-axis. Because the y-axis does not start at 0, only bins with a statistical significance score greater than approximately 3 are shown. This visual helps quickly visualize the statistically significant regions, which would be inferred to the genomic rearrangement partners with the target gene. Further, colored in purple are the significance scores at certain genomic bins whereby the significance scores in those bins is corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

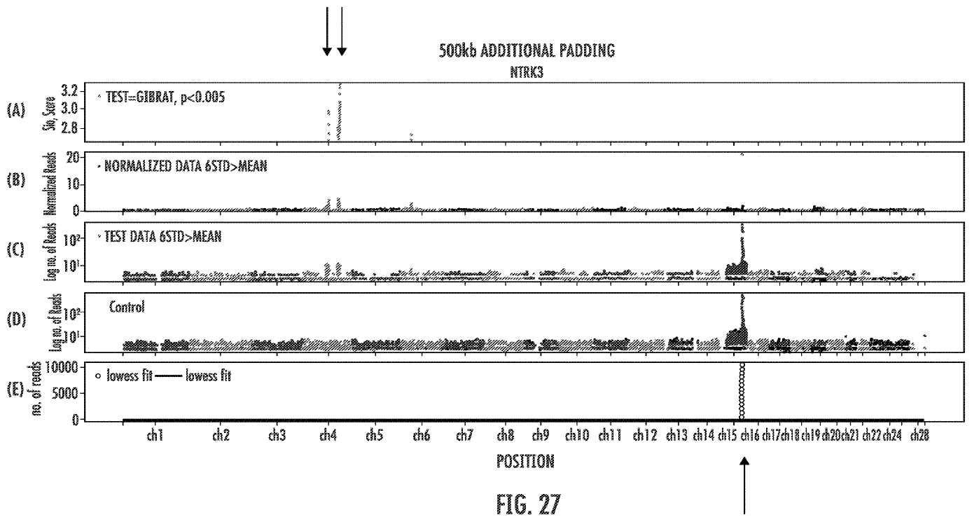

FIG. 27 shows Manhattan plots from experiment(s) where Arima Capture-HiC was performed using the 500 kb additional padding probe design set in accordance with an embodiment of the invention, using a cancer sample with a known breakpoint adjacent to the NTRK3 gene, and whereby the probe design set contains probes targeting the NTRK3 gene (a Group 1 gene in the probe design sets). Arima Capture-HiC was also performed on a karyotypically normal lymphoblast cell line, GM12878. The data were analyzed in several ways and visualized as Manhattans plots in Rows A-E, and described in greater detail below. Note all instances below referring to the “target gene” refer to NTRK3.

Row A—Manhattan plot showing data from the cancer sample, showing the data from Panel B except transformed into a statistical significance score for the y-axis. Because the y-axis does not start at 0, only bins with a statistical significance score greater than approximately 2.5 are shown. This visual helps quickly visualize the statistically significant regions, which would be inferred to the genomic rearrangement partners with the target gene. Further, colored in purple are the significance scores at certain genomic bins whereby the significance scores in those bins is corresponds to a p value less than 0.005.

Row B—Manhattan plot showing data from the cancer sample, showing the normalized counts of Capture-HiC read-pairs where one read-end originates from the probed region associated with the target gene, and the other read-end maps to any genomic bin across the genome. Normalization occurs by dividing the raw counts from the cancer sample (the data from Panel C) by the lowess fit data from the karyotypically normal GM12878 sample (the data from panel E). Colored in purple are the normalized counts at certain genomic bins whereby the normalized counts in those bins is greater than 6 standard deviations from the mean number of normalized counts across all bins.

Row C—Manhattan plot showing data from the cancer sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene. Colored in purple are the raw counts at certain genomic bins whereby the raw counts in those bins is greater than 6 standard deviations from the mean number of raw counts across all bins. While this type of analysis isn't used in the preferred method of identifying genomic rearrangement events in Panels A or B, it helps underscore the benefit of using the normalization methods of panels A and B to identify rearrangements and minimize false positives that may occur when using the simple intra-sample standard deviation cutoff based method of panel C.

Row D—Manhattan plot showing data from karyotypically normal GM12878 sample, showing the raw counts of Capture-HiC read-pairs where one read-end originates from the probed region associated with the target gene, and the other read-end maps to any genomic bin across the genome. This effectively plots, for each bin along the genome, how many Capture-HiC interactions were captured with the probed region associated with the target gene.

Row E—Lowess fit model using the data from Panel D.

Below row E is a red arrow which points to the genomic location of the target gene, and above row A is a blue arrow(s) pointing to the genomic location of the genomic rearrangement partner(s) with the target gene.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described, the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

APPENDIX 1

| Cancer Gene | | | | |
|---|---|---|---|---|
| Name | Chromosome | Start | end | strand |
| A1CF | chr10 | 50799409 | 50885675 | − |
| ABI1 | chr10 | 26746593 | 26861087 | − |
| ABI3BP | chr3 | 100749156 | 100993515 | − |
| ABL1 | chr9 | 130713016 | 130887675 | + |
| ABL2 | chr1 | 179099330 | 179229684 | − |
| ABRAXAS1 | chr4 | 83459517 | 83523348 | − |
| ACKR3 | chr2 | 236567787 | 236582354 | + |
| ACSL3 | chr2 | 222860942 | 222944639 | + |
| ACSL6 | chr5 | 131949973 | 132012243 | − |
| ACTB | chr7 | 5526409 | 5563902 | − |
| ACTG1 | chr17 | 81509413 | 81523847 | − |
| ACVR1 | chr2 | 157736251 | 157876330 | − |
| ACVR1B | chr12 | 51951699 | 51997078 | + |
| ACVR2A | chr2 | 147844517 | 147930826 | + |
| ADAMTS20 | chr12 | 43353866 | 43552203 | − |
| ADGRA2 | chr8 | 37784191 | 37844896 | + |
| ADGRB3 | chr6 | 68635282 | 69390571 | + |
| ADGRF5 | chr6 | 46852522 | 46954943 | − |
| ADGRL3 | chr4 | 61200326 | 62078335 | + |
| AFDN | chr6 | 167826911 | 167972023 | + |
| AFF1 | chr4 | 86935002 | 87141039 | + |
| AFF3 | chr2 | 99545419 | 100192428 | − |
| AFF4 | chr5 | 132875395 | 132963634 | − |
| AGO1 | chr1 | 35869808 | 35930532 | + |
| AGO2 | chr8 | 140520156 | 140635633 | − |
| AICDA | chr12 | 8602170 | 8612867 | − |
| AJUBA | chr14 | 22971177 | 22982551 | − |
| AKAP9 | chr7 | 91940840 | 92110673 | + |
| AKT1 | chr14 | 104769349 | 104795751 | − |
| AKT2 | chr19 | 40230317 | 40285536 | − |
| AKT3 | chr1 | 243488233 | 243851079 | − |
| ALB | chr4 | 73397114 | 73421482 | + |
| ALDH2 | chr12 | 111766887 | 111817532 | + |
| ALK | chr2 | 29192774 | 29921586 | − |
| ALOX12B | chr17 | 8072636 | 8087716 | − |
| AMER1 | chrX | 64185117 | 64205708 | − |
| ANK1 | chr8 | 41653220 | 41896762 | − |
| ANKRD11 | chr16 | 89267630 | 89490561 | − |
| ANKRD24 | chr19 | 4183354 | 4224814 | + |
| ANKRD26 | chr10 | 26973793 | 27100494 | − |
| ANKRD30BL | chr2 | 132147591 | 132257969 | − |
| APC | chr5 | 112707498 | 112846239 | + |
| APH1A | chr1 | 150265399 | 150269580 | − |
| APLNR | chr11 | 57233577 | 57237235 | − |
| APOBEC3B | chr22 | 38982347 | 38992804 | + |
| AR | chrX | 67544021 | 67730619 | + |
| ARAF | chrX | 47561205 | 47571908 | + |
| ARFGAP3 | chr22 | 42796502 | 42858106 | − |
| ARFRP1 | chr20 | 63698642 | 63708025 | − |
| ARHGAP26 | chr5 | 142770377 | 143229011 | + |
| ARHGAP35 | chr19 | 46860997 | 47005077 | + |
| ARHGAP5 | chr14 | 32076114 | 32159728 | + |
| ARHGAP6 | chrX | 11117651 | 11665920 | − |
| ARHGEF10 | chr8 | 1823926 | 1958641 | + |
| ARHGEF10L | chr1 | 17539698 | 17697874 | + |
| ARHGEF12 | chr11 | 120336413 | 120489937 | + |
| ARHGEF28 | chr5 | 73626158 | 73941993 | + |
| ARID1A | chr1 | 26693236 | 26782104 | + |
| ARID1B | chr6 | 156776020 | 157210779 | + |
| ARID2 | chr12 | 45729706 | 45908040 | + |
| ARID3A | chr19 | 925781 | 975939 | + |
| ARID3B | chr15 | 74541206 | 74598131 | + |
| ARID3C | chr9 | 34621379 | 34628086 | − |
| ARID4A | chr14 | 58298504 | 58373887 | + |
| ARID4B | chr1 | 235131634 | 235328219 | − |
| ARID5A | chr2 | 96536743 | 96552634 | + |
| ARID5B | chr10 | 61901684 | 62096944 | + |
| ARNT | chr1 | 150809713 | 150876708 | − |
| ASB13 | chr10 | 5638867 | 5666595 | − |
| ASH1L | chr1 | 155335268 | 155563162 | − |
| ASMTL | chrX | 1403139 | 1453762 | − |
| ASPSCR1 | chr17 | 81976807 | 82017406 | + |
| ASXL1 | chr20 | 32358330 | 32439319 | + |
| ASXL2 | chr2 | 25733753 | 25878487 | − |
| ATCAY | chr19 | 3879864 | 3928082 | + |
| ATF1 | chr12 | 50763710 | 50821162 | + |
| ATG5 | chr6 | 106045423 | 106325791 | − |
| ATIC | chr2 | 215311956 | 215349773 | + |
| ATM | chr11 | 108223044 | 108369102 | + |
| ATP1A1 | chr1 | 116372668 | 116410261 | + |
| ATP2B3 | chrX | 153517642 | 153582939 | + |
| ATP5PD | chr17 | 75038863 | 75046985 | − |
| ATP6AP1 | chrX | 154428633 | 154436516 | + |
| ATP6V1B2 | chr8 | 20197381 | 20226819 | + |
| ATR | chr3 | 142449007 | 142578733 | − |
| ATRX | chrX | 77504880 | 77786233 | − |
| ATXN2 | chr12 | 111443485 | 111599676 | − |
| ATXN7 | chr3 | 63863155 | 64003462 | + |
| AURKA | chr20 | 56369389 | 56392337 | − |
| AURKB | chr17 | 8204733 | 8210600 | − |
| AURKC | chr19 | 57230802 | 57235548 | + |
| AUTS2 | chr7 | 69598296 | 70793506 | + |
| AXIN1 | chr16 | 287440 | 352723 | − |
| AXIN2 | chr17 | 65528563 | 65561648 | − |
| AXL | chr19 | 41219223 | 41261766 | + |
| B2M | chr15 | 44711487 | 44718851 | + |
| BABAM1 | chr19 | 17267376 | 17281249 | + |
| BACH2 | chr6 | 89926528 | 90296908 | − |
| BAP1 | chr3 | 52401008 | 52410008 | − |
| BARD1 | chr2 | 214725646 | 214809683 | − |
| BATF3 | chr1 | 212686417 | 212699840 | − |
| BAX | chr19 | 48954815 | 48961798 | + |
| BAZ1A | chr14 | 34752731 | 34875647 | − |
| BBC3 | chr19 | 47220822 | 47232766 | − |
| BCL10 | chr1 | 85265776 | 85276632 | − |
| BCL11A | chr2 | 60450520 | 60554467 | − |
| BCL11B | chr14 | 99169287 | 99272197 | − |
| BCL2 | chr18 | 63123346 | 63320128 | − |
| BCL2A1 | chr15 | 79960892 | 79971196 | − |
| BCL2L1 | chr20 | 31664452 | 31723989 | − |
| BCL2L11 | chr2 | 111119378 | 111168445 | + |
| BCL2L12 | chr19 | 49665142 | 49673916 | + |
| BCL2L2 | chr14 | 23298790 | 23311751 | + |
| BCL3 | chr19 | 44747705 | 44760044 | + |
| BCL6 | chr3 | 187721377 | 187745725 | − |
| BCL7A | chr12 | 122019422 | 122062044 | + |
| BCL9 | chr1 | 147541501 | 147626216 | + |
| BCL9L | chr11 | 118893875 | 118925926 | − |
| BCLAF1 | chr6 | 136256627 | 136289851 | − |
| BCOR | chrX | 40049815 | 40177329 | − |
| BCORL1 | chrX | 129981107 | 130058071 | + |
| BCR | chr22 | 23179704 | 23318037 | + |
| BIRC2 | chr11 | 102347211 | 102378670 | + |
| BIRC3 | chr11 | 102317450 | 102339403 | + |
| BIRC5 | chr17 | 78214186 | 78225636 | + |
| BIRC6 | chr2 | 32357028 | 32618899 | + |
| BLM | chr15 | 90717346 | 90816166 | + |
| BLNK | chr10 | 96189171 | 96271587 | − |
| BLOC1S3 | chr19 | 45178784 | 45216933 | + |
| BMF | chr15 | 40087890 | 40108892 | − |
| BMP5 | chr6 | 55753653 | 55875590 | − |
| BMP7 | chr20 | 57168753 | 57266641 | − |
| BMPR1A | chr10 | 86756601 | 86932825 | + |
| BOD1L1 | chr4 | 13568738 | 13627725 | − |
| BRAF | chr7 | 140719327 | 140924929 | − |
| BRCA1 | chr17 | 43044295 | 43170245 | − |
| BRCA2 | chr13 | 32315086 | 32400268 | + |
| BRD3 | chr9 | 134030305 | 134068535 | − |
| BRD4 | chr19 | 15235519 | 15332545 | − |
| BRINP3 | chr1 | 190097658 | 190478404 | − |
| BRIP1 | chr17 | 61679139 | 61863559 | − |
| BRSK1 | chr19 | 55282072 | 55312562 | + |
| BTG1 | chr12 | 92140278 | 92145846 | − |
| BTG2 | chr1 | 203305491 | 203309602 | + |
| BTK | chrX | 101349447 | 101390796 | − |
| BTLA | chr3 | 112463966 | 112499472 | − |
| BUB1B | chr15 | 40161023 | 40221123 | + |
| C15orf65 | chr15 | 55408495 | 55418798 | + |
| CACNA1D | chr3 | 53328963 | 53813733 | + |
| CACNA1E | chr1 | 181317690 | 181808084 | + |
| CAD | chr2 | 27217369 | 27243943 | + |
| CALR | chr19 | 12938578 | 12944489 | + |
| CAMTA1 | chr1 | 6785454 | 7769706 | + |
| CANT1 | chr17 | 78991716 | 79009867 | − |
| CARD11 | chr7 | 2906142 | 3043867 | − |
| CARM1 | chr19 | 10871553 | 10923075 | + |
| CARS1 | chr11 | 3000922 | 3057613 | − |
| CASP3 | chr4 | 184627696 | 184649509 | − |
| CASP8 | chr2 | 201233443 | 201287711 | + |
| CASP9 | chr1 | 15490832 | 15526534 | − |
| CBFA2T3 | chr16 | 88874858 | 88977207 | − |
| CBFB | chr16 | 67028984 | 67101058 | + |
| CBL | chr11 | 119206298 | 119313926 | + |
| CBLB | chr3 | 105655461 | 105869552 | − |
| CBLC | chr19 | 44777869 | 44800652 | + |
| CCDC50 | chr3 | 191329085 | 191398659 | + |
| CCDC6 | chr10 | 59788747 | 59906556 | − |
| CCN6 | chr6 | 112054075 | 112069686 | + |
| CCNB1IP1 | chr14 | 20311368 | 20333312 | − |
| CCNB3 | chrX | 50202713 | 50351914 | + |
| CCNC | chr6 | 99542387 | 99568825 | − |
| CCND1 | chr11 | 69641156 | 69654474 | + |
| CCND2 | chr12 | 4269771 | 4305353 | + |
| CCND3 | chr6 | 41934934 | 42050357 | − |
| CCNE1 | chr19 | 29811991 | 29824312 | + |
| CCNQ | chrX | 153587925 | 153600045 | − |
| CCR4 | chr3 | 32951644 | 32956349 | + |
| CCR7 | chr17 | 40553769 | 40565472 | − |
| CCT6B | chr17 | 34927859 | 34981078 | − |
| CD209 | chr19 | 7739993 | 7747564 | − |
| CD22 | chr19 | 35319261 | 35347361 | + |
| CD274 | chr9 | 5450503 | 5470566 | + |
| CD276 | chr15 | 73683966 | 73714514 | + |
| CD28 | chr2 | 203706475 | 203738912 | + |
| CD36 | chr7 | 80369575 | 80679277 | + |
| CD44 | chr11 | 35138882 | 35232402 | + |
| CD58 | chr1 | 116514534 | 116571039 | − |
| CD63 | chr12 | 55725323 | 55729707 | − |
| CD70 | chr19 | 6583183 | 6604103 | − |
| CD74 | chr5 | 150400041 | 150412969 | − |
| CD79A | chr19 | 41877279 | 41881372 | + |
| CD79B | chr17 | 63928740 | 63932336 | − |
| CD83 | chr6 | 14117256 | 14136918 | + |
| CDC25A | chr3 | 48157146 | 48188417 | − |
| CDC25C | chr5 | 138285269 | 138338355 | − |
| CDC42 | chr1 | 22025511 | 22101360 | + |
| CDC73 | chr1 | 193121983 | 193254815 | + |
| CDH1 | chr16 | 68737292 | 68835537 | + |
| CDH10 | chr5 | 24487100 | 24644978 | − |
| CDH11 | chr16 | 64943753 | 65126112 | − |
| CDH17 | chr8 | 94127162 | 94217303 | − |
| CDH2 | chr18 | 27932879 | 28177946 | − |
| CDH20 | chr18 | 61333430 | 61555779 | + |
| CDH23 | chr10 | 71396920 | 71815947 | + |
| CDH5 | chr16 | 66366622 | 66404784 | + |
| CDK12 | chr17 | 39461486 | 39564907 | + |
| CDK4 | chr12 | 57747727 | 57756013 | − |
| CDK6 | chr7 | 92604921 | 92836573 | − |
| CDK8 | chr13 | 26254104 | 26405238 | + |
| CDKN1A | chr6 | 36676460 | 36687337 | + |
| CDKN1B | chr12 | 12685498 | 12722369 | + |
| CDKN2A | chr9 | 21967752 | 21995301 | − |
| CDKN2B | chr9 | 22002903 | 22009305 | − |
| CDKN2C | chr1 | 50960745 | 50974634 | + |
| CDX2 | chr13 | 27960918 | 27969315 | − |
| CEBPA | chr19 | 33299934 | 33302534 | − |
| CEBPD | chr8 | 47736913 | 47738164 | − |
| CEBPE | chr14 | 23117306 | 23119255 | − |
| CEBPG | chr19 | 33373685 | 33382686 | + |
| CENPA | chr2 | 26764289 | 26801067 | + |
| CEP43 | chr6 | 166999317 | 167094789 | + |
| CEP89 | chr19 | 32875925 | 32971991 | − |
| CHCHD7 | chr8 | 56211686 | 56218809 | + |
| CHD1 | chr5 | 98853985 | 98929007 | − |
| CHD2 | chr15 | 92900189 | 93027996 | + |
| CHD4 | chr12 | 6570082 | 6614524 | − |
| CHD5 | chr1 | 6101787 | 6180321 | − |
| CHD7 | chr8 | 60678740 | 60868028 | + |
| CHEK1 | chr11 | 125625163 | 125676255 | + |
| CHEK2 | chr22 | 28687743 | 28742422 | − |
| CHIC2 | chr4 | 54009789 | 54064605 | − |
| CHN1 | chr2 | 174798809 | 175005381 | − |
| CHST11 | chr12 | 104455295 | 104762014 | + |
| CIC | chr19 | 42268537 | 42295797 | + |
| CIITA | chr16 | 10866222 | 10943021 | + |
| CILK1 | chr6 | 53001279 | 53061824 | − |
| CKS1B | chr1 | 154974653 | 154979251 | + |
| CLIP1 | chr12 | 122271432 | 122422669 | − |
| CLP1 | chr11 | 57648188 | 57661865 | + |
| CLTC | chr17 | 59619689 | 59696956 | + |
| CLTCL1 | chr22 | 19179473 | 19291719 | − |
| CMPK1 | chr1 | 47333790 | 47378839 | + |
| CMTR2 | chr16 | 71281389 | 71289715 | − |
| CNBD1 | chr8 | 86866415 | 87615219 | + |
| CNBP | chr3 | 129167827 | 129183922 | − |
| CNOT3 | chr19 | 54137749 | 54155681 | + |
| CNTNAP2 | chr7 | 146116002 | 148420998 | + |
| CNTRL | chr9 | 121074753 | 121177610 | + |
| COL1A1 | chr17 | 50184101 | 50201632 | − |
| COL2A1 | chr12 | 47972967 | 48004554 | − |
| COL3A1 | chr2 | 188974373 | 189012746 | + |
| COP1 | chr1 | 175944831 | 176207286 | − |
| COX10 | chr17 | 14069490 | 14231736 | + |
| COX5B | chr2 | 97646062 | 97648383 | + |
| COX6C | chr8 | 99873200 | 99893707 | − |
| CPEB3 | chr10 | 92046692 | 92291078 | − |
| CPS1 | chr2 | 210477682 | 210679107 | + |
| CRBN | chr3 | 3144628 | 3179727 | − |
| CREB1 | chr2 | 207529737 | 207605988 | + |
| CREB3L1 | chr11 | 46277662 | 46321409 | + |
| CREB3L2 | chr7 | 137874979 | 138002086 | − |
| CREBBP | chr16 | 3725054 | 3880713 | − |
| CRKL | chr22 | 20917407 | 20953747 | + |
| CRLF2 | chrX | 1187549 | 1212723 | − |
| CRNKL1 | chr20 | 20034368 | 20056046 | − |
| CRTC1 | chr19 | 18683678 | 18782333 | + |
| CRTC3 | chr15 | 90529923 | 90645345 | + |
| CSDE1 | chr1 | 114716913 | 114758676 | − |
| CSF1 | chr1 | 109910242 | 109930992 | + |
| CSF1R | chr5 | 150053291 | 150113372 | − |
| CSF3R | chr1 | 36466043 | 36483278 | − |
| CSMD3 | chr8 | 112222928 | 113436939 | − |
| CSNK2B | chr6 | 31665227 | 31670343 | + |
| CTCF | chr16 | 67562467 | 67639177 | + |
| CTDNEP1 | chr17 | 7243591 | 7252491 | − |
| CTLA4 | chr2 | 203867771 | 203873965 | + |
| CTNNA1 | chr5 | 138610967 | 138935034 | + |
| CTNNA2 | chr2 | 79185231 | 80648861 | + |
| CTNNB1 | chr3 | 41194741 | 41260096 | + |
| CTNND1 | chr11 | 57753243 | 57819546 | + |
| CTNND2 | chr5 | 10971836 | 11904446 | − |
| CTR9 | chr11 | 10751246 | 10801625 | + |
| CUL3 | chr2 | 224470150 | 224585397 | − |
| CUL4A | chr13 | 113208193 | 113267108 | + |
| CUX1 | chr7 | 101815904 | 102283958 | + |
| CXCR4 | chr2 | 136114349 | 136118149 | − |
| CYB5R2 | chr11 | 7665100 | 7677222 | − |
| CYLD | chr16 | 50742050 | 50801935 | + |
| CYP17A1 | chr10 | 102830531 | 102837472 | − |
| CYP19A1 | chr15 | 51208057 | 51338601 | − |
| CYP2C19 | chr10 | 94762681 | 94855547 | + |
| CYP2C8 | chr10 | 95036772 | 95069497 | − |
| CYP2D6 | chr22 | 42126499 | 42130865 | − |
| CYSLTR2 | chr13 | 48653711 | 48711226 | + |
| DAXX | chr6 | 33318558 | 33323016 | − |
| DCAF12L2 | chrX | 126163499 | 126166289 | − |
| DCC | chr18 | 52340197 | 53535903 | + |
| DCK | chr4 | 70992538 | 71030914 | + |
| DCTN1 | chr2 | 74361154 | 74392087 | − |
| DCUN1D1 | chr3 | 182938074 | 182985953 | − |
| DDB2 | chr11 | 47214465 | 47239217 | + |
| DDIT3 | chr12 | 57516588 | 57521737 | − |
| DDR1 | chr6 | 30876421 | 30900156 | + |
| DDR2 | chr1 | 162631373 | 162787405 | + |
| DDX10 | chr11 | 108665069 | 108940927 | + |
| DDX3X | chrX | 41333348 | 41364472 | + |
| DDX4 | chr5 | 55738017 | 55817157 | + |
| DDX41 | chr5 | 177511577 | 177516961 | − |
| DDX5 | chr17 | 64498254 | 64508199 | − |
| DDX6 | chr11 | 118747763 | 118791164 | − |
| DEK | chr6 | 18223860 | 18264548 | − |
| DENND3 | chr8 | 141117278 | 141195808 | + |
| DGCR8 | chr22 | 20080232 | 20111877 | + |
| DHX15 | chr4 | 24517441 | 24584554 | − |
| DICER1 | chr14 | 95086228 | 95158010 | − |
| DIS3 | chr13 | 72752169 | 72782096 | − |
| DLEU1 | chr13 | 50082169 | 50906856 | + |
| DNAH9 | chr17 | 11598470 | 11969748 | + |
| DNAJB1 | chr19 | 14514769 | 14560391 | − |
| DNM2 | chr19 | 10718055 | 10833488 | + |
| DNMT1 | chr19 | 10133342 | 10231286 | − |
| DNMT3A | chr2 | 25227855 | 25342590 | − |
| DNMT3B | chr20 | 32762385 | 32809356 | + |
| DNTT | chr10 | 96304409 | 96338564 | + |
| DOT1L | chr19 | 2163933 | 2232578 | + |
| DPH3 | chr3 | 16257061 | 16264969 | − |
| DPYD | chr1 | 97077743 | 97995000 | − |
| DROSHA | chr5 | 31400494 | 31532196 | − |
| DST | chr6 | 56457987 | 56954830 | − |
| DTX1 | chr12 | 113056709 | 113098028 | + |
| DUSP2 | chr2 | 96143169 | 96145440 | − |
| DUSP22 | chr6 | 291630 | 351355 | + |
| DUSP4 | chr8 | 29333064 | 29350684 | − |
| DUSP9 | chrX | 153642492 | 153651326 | + |
| DUX4L1 | chr4 | 190084412 | 190085686 | + |
| E2F2 | chr1 | 23506438 | 23531233 | − |
| E2F3 | chr6 | 20401879 | 20493714 | + |
| EBF1 | chr5 | 158695916 | 159099916 | − |
| ECT2L | chr6 | 138795911 | 138904070 | + |
| EED | chr11 | 86244753 | 86278813 | + |
| EGF | chr4 | 109912883 | 110013766 | + |
| EGFL7 | chr9 | 136658856 | 136672678 | + |
| EGFR | chr7 | 55019017 | 55211628 | + |
| EGR1 | chr5 | 138465479 | 138469303 | + |
| EIF1AX | chrX | 20124525 | 20141838 | − |
| EIF3E | chr8 | 108162787 | 108443496 | − |
| EIF4A1 | chr17 | 7572824 | 7579006 | + |
| EIF4A2 | chr3 | 186783205 | 186789897 | + |
| EIF4E | chr4 | 98879276 | 98929133 | − |
| ELF3 | chr1 | 202007945 | 202017183 | + |
| ELF4 | chrX | 130063955 | 130110716 | − |
| ELK4 | chr1 | 205597556 | 205632011 | − |
| ELL | chr19 | 18442663 | 18522116 | − |
| ELN | chr7 | 74027789 | 74069907 | + |
| ELOC | chr8 | 73939169 | 73972287 | − |
| ELP2 | chr18 | 36129444 | 36180557 | + |
| ELP6 | chr3 | 47495640 | 47513712 | − |
| EML4 | chr2 | 42169353 | 42332548 | + |
| EMSY | chr11 | 76444923 | 76553025 | + |
| ENTPD1 | chr10 | 95711779 | 95877266 | + |
| EP300 | chr22 | 41092592 | 41180077 | + |
| EP400 | chr12 | 131949942 | 132080460 | + |
| EPAS1 | chr2 | 46293667 | 46386697 | + |
| EPC1 | chr10 | 32267751 | 32378798 | − |
| EPCAM | chr2 | 47345158 | 47387601 | + |
| EPHA2 | chr1 | 16124337 | 16156069 | − |
| EPHA3 | chr3 | 89107621 | 89482134 | + |
| EPHA5 | chr4 | 65319563 | 65670495 | − |
| EPHA7 | chr6 | 93240020 | 93419687 | − |
| EPHB1 | chr3 | 134795260 | 135260467 | + |
| EPHB4 | chr7 | 100802565 | 100827523 | − |
| EPHB6 | chr7 | 142855061 | 142871094 | + |
| EPOR | chr19 | 11377207 | 11384342 | − |
| EPS15 | chr1 | 51354263 | 51519266 | − |
| ERBB2 | chr17 | 39687914 | 39730426 | + |
| ERBB3 | chr12 | 56076799 | 56103505 | + |
| ERBB4 | chr2 | 211375717 | 212538841 | − |
| ERC1 | chr12 | 990509 | 1495933 | + |
| ERCC1 | chr19 | 45407334 | 45478828 | − |
| ERCC2 | chr19 | 45349837 | 45370918 | − |
| ERCC3 | chr2 | 127257290 | 127294166 | − |
| ERCC4 | chr16 | 13920138 | 13952348 | + |
| ERCC5 | chr13 | 102845831 | 102875995 | + |
| ERF | chr19 | 42247569 | 42255128 | − |
| ERG | chr21 | 38380027 | 38661780 | − |
| ERRFI1 | chr1 | 8004404 | 8026309 | − |
| ESCO2 | chr8 | 27771949 | 27812640 | + |
| ESR1 | chr6 | 151656691 | 152129619 | + |
| ESRRA | chr11 | 64305497 | 64316743 | + |
| ETAA1 | chr2 | 67397322 | 67412089 | + |
| ETNK1 | chr12 | 22625075 | 22690665 | + |
| ETS1 | chr11 | 128458761 | 128587558 | − |
| ETV1 | chr7 | 13891229 | 13991425 | − |
| ETV4 | chr17 | 43527844 | 43579620 | − |
| ETV5 | chr3 | 186046314 | 186110318 | − |
| ETV6 | chr12 | 11649674 | 11895377 | + |
| EVA1C | chr21 | 32412006 | 32515397 | + |
| EWSR1 | chr22 | 29268009 | 29300525 | + |
| EXOC2 | chr6 | 485154 | 693139 | − |
| EXOC7 | chr17 | 76081016 | 76121576 | − |
| EXOSC6 | chr16 | 70246778 | 70251940 | − |
| EXT1 | chr8 | 117794490 | 118111826 | − |
| EXT2 | chr11 | 44095648 | 44251962 | + |
| EZH1 | chr17 | 42700275 | 42745049 | − |
| EZH2 | chr7 | 148807257 | 148884321 | − |
| EZHIP | chrX | 51406948 | 51408843 | + |
| EZR | chr6 | 158765741 | 158819368 | − |
| FAF1 | chr1 | 50437028 | 50960267 | − |
| FAM131B | chr7 | 143353400 | 143362770 | − |
| FAM135B | chr8 | 138130023 | 138497261 | − |
| FAM216A | chr12 | 110468415 | 110490385 | + |
| FAM47C | chrX | 37008366 | 37011664 | + |
| FANCA | chr16 | 89737549 | 89816657 | − |
| FANCC | chr9 | 95099054 | 95426796 | − |
| FANCD2 | chr3 | 10026370 | 10101932 | + |
| FANCE | chr6 | 35452338 | 35467102 | + |
| FANCF | chr11 | 22622533 | 22625823 | − |
| FANCG | chr9 | 35073835 | 35079942 | − |
| FANCL | chr2 | 58159243 | 58241372 | − |
| FAS | chr10 | 88990531 | 89017059 | + |
| FAT1 | chr4 | 186587794 | 186726722 | − |
| FAT3 | chr11 | 92224818 | 92896473 | + |
| FAT4 | chr4 | 125314918 | 125492932 | + |
| FBLN2 | chr3 | 13549125 | 13638422 | + |
| FBXO11 | chr2 | 47789316 | 47906498 | − |
| FBXO31 | chr16 | 87326987 | 87392142 | − |
| FBXW4 | chr10 | 101610664 | 101695295 | − |
| FBXW7 | chr4 | 152320544 | 152536092 | − |
| FCGR2B | chr1 | 161663147 | 161678654 | + |
| FCHO1 | chr19 | 17747718 | 17788568 | + |
| FCRL4 | chr1 | 157573747 | 157598085 | − |
| FEN1 | chr11 | 61792911 | 61797238 | + |
| FES | chr15 | 90883695 | 90895776 | + |
| FEV | chr2 | 218981087 | 218985184 | − |
| FGF1 | chr5 | 142592178 | 142698070 | − |
| FGF10 | chr5 | 44300247 | 44389706 | − |
| FGF12 | chr3 | 192139390 | 192767764 | − |
| FGF14 | chr13 | 101710804 | 102402457 | − |
| FGF19 | chr11 | 69698238 | 69704022 | − |
| FGF23 | chr12 | 4368227 | 4379712 | − |
| FGF3 | chr11 | 69809968 | 69819416 | − |
| FGF4 | chr11 | 69771022 | 69775341 | − |
| FGF6 | chr12 | 4428155 | 4445815 | − |
| FGFR1 | chr8 | 38400215 | 38468834 | − |
| FGFR2 | chr10 | 121478332 | 121598458 | − |
| FGFR3 | chr4 | 1793293 | 1808872 | + |
| FGFR4 | chr5 | 177086905 | 177098144 | + |
| FGR | chr1 | 27612064 | 27635185 | − |
| FH | chr1 | 241497511 | 241519799 | − |
| FHIT | chr3 | 59747277 | 61251459 | − |
| FIP1L1 | chr4 | 53377641 | 53460862 | + |
| FKBP9 | chr7 | 32957404 | 33006930 | + |
| FLCN | chr17 | 17212212 | 17237188 | − |
| FLI1 | chr11 | 128686535 | 128813267 | + |
| FLNA | chrX | 154348524 | 154374634 | − |
| FLT1 | chr13 | 28300346 | 28495145 | − |
| FLT3 | chr13 | 28003274 | 28100592 | − |
| FLT4 | chr5 | 180601506 | 180649624 | − |
| FLYWCH1 | chr16 | 2911931 | 2951208 | + |
| FN1 | chr2 | 215360440 | 215436073 | − |
| FNBP1 | chr9 | 129887187 | 130043194 | − |
| FOS | chr14 | 75278826 | 75282230 | + |
| FOSB | chr19 | 45467995 | 45475179 | + |
| FOXA1 | chr14 | 37589552 | 37596059 | − |
| FOXF1 | chr16 | 86510527 | 86515422 | + |
| FOXL2 | chr3 | 138944224 | 138947137 | − |
| FOXO1 | chr13 | 40555667 | 40666641 | − |
| FOXO3 | chr6 | 108559835 | 108684774 | + |
| FOXO4 | chrX | 71095851 | 71103532 | + |
| FOXP1 | chr3 | 70954708 | 71583989 | − |
| FOXP4 | chr6 | 41546381 | 41602384 | + |
| FOXR1 | chr11 | 118971712 | 118981287 | + |
| FOXR2 | chrX | 55623400 | 55626192 | + |
| FRG2C | chr3 | 75664328 | 75667220 | + |
| FRS2 | chr12 | 69470349 | 69579793 | + |
| FSTL3 | chr19 | 676392 | 683392 | + |
| FUBP1 | chr1 | 77944055 | 77979110 | − |
| FURIN | chr15 | 90868588 | 90883564 | + |
| FUS | chr16 | 31180138 | 31191605 | + |
| FUT8 | chr14 | 65410592 | 65744121 | + |
| FYN | chr6 | 111660332 | 111873452 | − |
| FZR1 | chr19 | 3506311 | 3538334 | + |
| G6PD | chrX | 154531391 | 154547572 | − |
| GAB1 | chr4 | 143336762 | 143474568 | + |
| GAB2 | chr11 | 78215293 | 78418348 | − |
| GABRA6 | chr5 | 161547063 | 161702593 | + |
| GADD45B | chr19 | 2476122 | 2478259 | + |
| GALNT13 | chr2 | 153871922 | 154453979 | + |
| GAS7 | chr17 | 9910606 | 10198606 | − |
| GATA1 | chrX | 48786562 | 48794311 | + |
| GATA2 | chr3 | 128479427 | 128493201 | − |
| GATA3 | chr10 | 8045378 | 8075198 | + |
| GATA4 | chr8 | 11676959 | 11760002 | + |
| GATA6 | chr18 | 22169589 | 22202528 | + |
| GDNF | chr5 | 37812677 | 37840041 | − |
| GID4 | chr17 | 18039408 | 18068405 | + |
| GIPR | chr19 | 45668221 | 45683722 | + |
| GLI1 | chr12 | 57459785 | 57472268 | + |
| GLIS2 | chr16 | 4314761 | 4339597 | + |
| GMPS | chr3 | 155870650 | 155944020 | + |
| GNA11 | chr19 | 3094362 | 3123999 | + |
| GNA12 | chr7 | 2728105 | 2844308 | − |
| GNA13 | chr17 | 65009289 | 65056740 | − |
| GNAI3 | chr1 | 109548615 | 109600195 | + |
| GNAQ | chr9 | 77716097 | 78031811 | − |
| GNAS | chr20 | 58839718 | 58911192 | + |
| GNB1 | chr1 | 1785285 | 1891117 | − |
| GOLGA5 | chr14 | 92794305 | 92839947 | + |
| GOPC | chr6 | 117560269 | 117602542 | − |
| GPC3 | chrX | 133535745 | 133985594 | − |
| GPC5 | chr13 | 91398621 | 92873682 | + |
| GPHN | chr14 | 66507407 | 67181803 | + |
| GPS2 | chr17 | 7311324 | 7315564 | − |
| GRB7 | chr17 | 39737927 | 39747291 | + |
| GREM1 | chr15 | 32718004 | 32745106 | + |
| GRIN2A | chr16 | 9753404 | 10182928 | − |
| GRM3 | chr7 | 86643909 | 86864879 | + |
| GRM8 | chr7 | 126438598 | 127253093 | − |
| GSE1 | chr16 | 85169525 | 85676204 | + |
| GSK3B | chr3 | 119821321 | 120094994 | − |
| GTF21 | chr7 | 74650231 | 74760692 | + |
| GTSE1 | chr22 | 46296870 | 46330810 | + |
| GUCY1A2 | chr11 | 106674019 | 107018476 | − |
| H1-2 | chr6 | 26055740 | 26056470 | − |
| H1-3 | chr6 | 26234212 | 26234987 | − |
| H1-4 | chr6 | 26156329 | 26157115 | + |
| H1-5 | chr6 | 27866792 | 27867588 | − |
| H2AC11 | chr6 | 27133042 | 27135291 | + |
| H2AC16 | chr6 | 27865317 | 27865798 | + |
| H2AC17 | chr6 | 27892699 | 27893185 | − |
| H2AC6 | chr6 | 26124145 | 26139116 | + |
| H2BC11 | chr6 | 27125897 | 27132795 | − |
| H2BC12 | chr6 | 27146361 | 27146855 | − |
| H2BC17 | chr6 | 27893425 | 27893891 | + |
| H2BC4 | chr6 | 26114873 | 26123926 | − |
| H2BC5 | chr6 | 26158146 | 26171349 | + |
| H2BC8 | chr6 | 26215159 | 26216692 | − |
| H3-3A | chr1 | 226061851 | 226072019 | + |
| H3-3B | chr17 | 75776434 | 75785893 | − |
| H3-4 | chr1 | 228424845 | 228425360 | − |
| H3-5 | chr12 | 31791185 | 31792298 | − |
| H3C1 | chr6 | 26020451 | 26020958 | + |
| H3C10 | chr6 | 27810064 | 27811300 | + |
| H3C11 | chr6 | 27871845 | 27872346 | − |
| H3C12 | chr6 | 27890315 | 27893106 | − |
| H3C13 | chr1 | 149813225 | 149813693 | − |
| H3C14 | chr1 | 149839538 | 149841193 | − |
| H3C2 | chr6 | 26031589 | 26032099 | − |
| H3C3 | chr6 | 26045384 | 26045869 | + |
| H3C4 | chr6 | 26196784 | 26197286 | − |
| H3C6 | chr6 | 26224199 | 26227473 | + |
| H3C7 | chr6 | 26250142 | 26250635 | − |
| H3C8 | chr6 | 26269405 | 26271815 | − |
| H4C9 | chr6 | 27138588 | 27139881 | + |
| HAUS3 | chr4 | 2078998 | 2242276 | − |
| HCAR1 | chr12 | 122726076 | 122730844 | − |
| HDAC1 | chr1 | 32292083 | 32333635 | + |
| HDAC4 | chr2 | 239048168 | 239401654 | − |
| HDAC7 | chr12 | 47782722 | 47833132 | − |
| HERPUD1 | chr16 | 56932142 | 56944864 | + |
| HEY1 | chr8 | 79762371 | 79767857 | − |
| HGF | chr7 | 81699010 | 81770438 | − |
| HIF1A | chr14 | 61695513 | 61748259 | + |
| HIF3A | chr19 | 46297042 | 46343433 | + |
| HIP1 | chr7 | 75533298 | 75738962 | − |
| HIRA | chr22 | 19330698 | 19447450 | − |
| HLA-A | chr6 | 29941260 | 29945884 | + |
| HLA-B | chr6 | 31353872 | 31357188 | − |
| HLA-C | chr6 | 31268749 | 31272130 | − |
| HLF | chr17 | 55264960 | 55325187 | + |
| HMGA1 | chr6 | 34236873 | 34246231 | + |
| HMGA2 | chr12 | 65824460 | 65966291 | + |
| HMGCL | chr1 | 23801885 | 23838620 | − |
| HMGN2P46 | chr15 | 45511136 | 45586290 | + |
| HNF1A | chr12 | 120978543 | 121002512 | + |
| HNRNPA2B1 | chr7 | 26171151 | 26201529 | − |
| HNRNPK | chr9 | 83968083 | 83980616 | − |
| HOOK3 | chr8 | 42896946 | 43030535 | + |
| HOXA10 | chr7 | 27170592 | 27180261 | − |
| HOXA11 | chr7 | 27181157 | 27185232 | − |
| HOXA13 | chr7 | 27193503 | 27200091 | − |
| HOXA3 | chr7 | 27106184 | 27152583 | − |
| HOXA9 | chr7 | 27162438 | 27175180 | − |
| HOXB13 | chr17 | 48724763 | 48728750 | − |
| HOXC11 | chr12 | 53973126 | 53977643 | + |
| HOXC13 | chr12 | 53938831 | 53946544 | + |
| HOXD11 | chr2 | 176104216 | 176109754 | + |
| HOXD13 | chr2 | 176092721 | 176095944 | + |
| HRAS | chr11 | 532242 | 537321 | − |
| HSD3B1 | chr1 | 119507198 | 119515054 | + |
| HSP90AA1 | chr14 | 102080742 | 102139699 | − |
| HSP90AB1 | chr6 | 44246166 | 44253888 | + |
| ICOSLG | chr21 | 44217014 | 44240966 | − |
| ID3 | chr1 | 23557926 | 23559501 | − |
| ID4 | chr6 | 19837370 | 19842197 | + |
| IDH1 | chr2 | 208236229 | 208266074 | − |
| IDH2 | chr15 | 90083045 | 90102477 | − |
| IFNGR1 | chr6 | 137197485 | 137219449 | − |
| IGF1 | chr12 | 102395874 | 102481744 | − |
| IGF1R | chr15 | 98648539 | 98964530 | + |
| IGF2 | chr11 | 2129112 | 2141238 | − |
| IGF2BP2 | chr3 | 185643130 | 185825042 | − |
| IGF2R | chr6 | 159969082 | 160113507 | + |
| IGHA1 | chr14 | 105703995 | 105708665 | − |
| IGHA2 | chr14 | 105583731 | 105588395 | − |
| IGHG1 | chr14 | 105736343 | 105743071 | − |
| IGHG2 | chr14 | 105639559 | 105644790 | − |
| IGHG3 | chr14 | 105764503 | 105771405 | − |
| IGHG4 | chr14 | 105620506 | 105626066 | − |
| IGHJ1 | chr14 | 105865407 | 105865458 | − |
| IGHJ2 | chr14 | 105865199 | 105865250 | − |
| IGHJ3 | chr14 | 105864587 | 105864635 | − |
| IGHJ4 | chr14 | 105864215 | 105864260 | − |
| IGHJ5 | chr14 | 105863814 | 105863862 | − |
| IGHJ6 | chr14 | 105863198 | 105863258 | − |
| IGHM | chr14 | 105851705 | 105856218 | − |
| IKBKB | chr8 | 42271302 | 42332460 | + |
| IKBKE | chr1 | 206470476 | 206496889 | + |
| IKZF1 | chr7 | 50304068 | 50405101 | + |
| IKZF2 | chr2 | 212999691 | 213152427 | − |
| IKZF3 | chr17 | 39757718 | 39864312 | − |
| IL10 | chr1 | 206767602 | 206774541 | − |
| IL16 | chr15 | 81159575 | 81314058 | + |
| IL2 | chr4 | 122451470 | 122456725 | − |
| IL21R | chr16 | 27402174 | 27452042 | + |
| IL3 | chr5 | 132060655 | 132063204 | + |
| IL6ST | chr5 | 55935095 | 55995022 | − |
| IL7R | chr5 | 35852695 | 35879603 | + |
| ING4 | chr12 | 6650301 | 6663142 | − |
| INHA | chr2 | 219569162 | 219575711 | + |
| INHBA | chr7 | 41667168 | 41705834 | − |
| INPP4A | chr2 | 98444854 | 98594392 | + |
| INPP4B | chr4 | 142023160 | 142847432 | − |
| INPP5D | chr2 | 233059967 | 233207903 | + |
| INPPL1 | chr11 | 72223701 | 72239147 | + |
| INSR | chr19 | 7112255 | 7294414 | − |
| IRAG2 | chr12 | 25004342 | 25108334 | + |
| IRF1 | chr5 | 132440440 | 132508719 | − |
| IRF2 | chr4 | 184387729 | 184474550 | − |
| IRF4 | chr6 | 391752 | 411443 | + |
| IRF8 | chr16 | 85899162 | 85922606 | + |
| IRS1 | chr2 | 226731312 | 226799820 | − |
| IRS2 | chr13 | 109752695 | 109786583 | − |
| IRS4 | chrX | 108719946 | 108736563 | − |
| ISX | chr22 | 35066136 | 35087387 | + |
| ITGA10 | chr1 | 145891208 | 145910111 | − |
| ITGA9 | chr3 | 37452115 | 37823507 | + |
| ITGAV | chr2 | 186590056 | 186680901 | + |
| ITGB2 | chr21 | 44885953 | 44931989 | − |
| ITGB3 | chr17 | 47253827 | 47313743 | + |
| ITK | chr5 | 157142933 | 157255185 | + |
| ITPKB | chr1 | 226631690 | 226739323 | − |
| JAK1 | chr1 | 64833229 | 65067754 | − |
| JAK2 | chr9 | 4984390 | 5129948 | + |
| JAK3 | chr19 | 17824780 | 17848071 | − |
| JARID2 | chr6 | 15246069 | 15522042 | + |
| JAZF1 | chr7 | 27830573 | 28180795 | − |
| JUN | chr1 | 58776845 | 58784048 | − |
| KAT6A | chr8 | 41929479 | 42051994 | − |
| KAT6B | chr10 | 74824927 | 75032624 | + |
| KAT7 | chr17 | 49788648 | 49835026 | + |
| KBTBD4 | chr11 | 47572197 | 47578976 | − |
| KCNJ5 | chr11 | 128891356 | 128921163 | + |
| KDM2B | chr12 | 121429096 | 121581023 | − |
| KDM4C | chr9 | 6720863 | 7175648 | + |
| KDM5A | chr12 | 280057 | 389320 | − |
| KDM5C | chrX | 53191321 | 53225422 | − |
| KDM6A | chrX | 44873188 | 45112779 | + |
| KDR | chr4 | 55078481 | 55125595 | − |
| KDSR | chr18 | 63327726 | 63367228 | − |
| KEAP1 | chr19 | 10486125 | 10503558 | − |
| KEL | chr7 | 142941114 | 142962363 | − |
| KIAA1549 | chr7 | 138831381 | 138981389 | − |
| KIF5B | chr10 | 32009015 | 32056425 | − |
| KIT | chr4 | 54657918 | 54740715 | + |
| KLF15 | chr3 | 126342635 | 126357408 | − |
| KLF2 | chr19 | 16324826 | 16328685 | + |
| KLF3 | chr4 | 38664197 | 38701517 | + |
| KLF4 | chr9 | 107484852 | 107490482 | − |
| KLF5 | chr13 | 73054976 | 73077541 | + |
| KLF6 | chr10 | 3775996 | 3785281 | − |
| KLHL6 | chr3 | 183487551 | 183555706 | − |
| KLK2 | chr19 | 50861568 | 50880567 | + |
| KMT2A | chr11 | 118436490 | 118526832 | + |
| KMT2B | chr19 | 35717973 | 35738878 | + |
| KMT2C | chr7 | 152134922 | 152436644 | − |
| KMT2D | chr12 | 49018978 | 49060794 | − |
| KMT5A | chr12 | 123383773 | 123409353 | + |
| KNL1 | chr15 | 40594020 | 40664342 | + |
| KNSTRN | chr15 | 40382721 | 40394246 | + |
| KRAS | chr12 | 25205246 | 25250936 | − |
| KSR2 | chr12 | 117453012 | 117968990 | − |
| KTN1 | chr14 | 55559072 | 55701526 | + |
| LAMP1 | chr13 | 113297239 | 113323672 | + |
| LARP4B | chr10 | 806914 | 931705 | − |
| LASP1 | chr17 | 38869859 | 38921770 | + |
| LATS1 | chr6 | 149658153 | 149718105 | − |
| LATS2 | chr13 | 20973036 | 21061586 | − |
| LCK | chr1 | 32251239 | 32286165 | + |
| LCP1 | chr13 | 46125920 | 46211871 | − |
| LEF1 | chr4 | 108047545 | 108168956 | − |
| LEPROTL1 | chr8 | 30095408 | 30177208 | + |
| LHFPL6 | chr13 | 39209116 | 39603528 | − |
| LIFR | chr5 | 38474668 | 38608354 | − |
| LIMD1 | chr3 | 45555394 | 45686341 | + |
| LMNA | chr1 | 156082573 | 156140081 | + |
| LMO1 | chr11 | 8224309 | 8268716 | − |
| LMO2 | chr11 | 33858576 | 33892076 | − |
| LPP | chr3 | 188153284 | 188890671 | + |
| LRIG3 | chr12 | 58872149 | 58920504 | − |
| LRP1B | chr2 | 140231423 | 142131016 | − |
| LRP5 | chr11 | 68312591 | 68449275 | + |
| LRP6 | chr12 | 12116025 | 12267044 | − |
| LRRK2 | chr12 | 40196744 | 40369285 | + |
| LSM14A | chr19 | 34172504 | 34229288 | + |
| LTB | chr6 | 31580525 | 31582522 | − |
| LTF | chr3 | 46435645 | 46485234 | − |
| LTK | chr15 | 41503637 | 41513887 | − |
| LUC7L2 | chr7 | 139340359 | 139423457 | + |
| LYL1 | chr19 | 13099033 | 13103161 | − |
| LYN | chr8 | 55879835 | 56014169 | + |
| LZTR1 | chr22 | 20982269 | 20999032 | + |
| LZTS1 | chr8 | 20246165 | 20303963 | − |
| MACC1 | chr7 | 20134655 | 20217404 | − |
| MAD2L2 | chr1 | 11674480 | 11691650 | − |
| MAF | chr16 | 79585843 | 79600737 | − |
| MAFB | chr20 | 40685848 | 40689236 | − |
| MAGEA1 | chrX | 153179284 | 153183880 | + |
| MAGED1 | chrX | 51803007 | 51902354 | + |
| MAGI1 | chr3 | 65353525 | 66038834 | − |
| MAL | chr2 | 95025677 | 95053992 | + |
| MALAT1 | chr11 | 65497688 | 65506516 | + |
| MALT1 | chr18 | 58671465 | 58754477 | + |
| MAML2 | chr11 | 95976598 | 96343195 | − |
| MAML3 | chr4 | 139716753 | 140154184 | − |
| MAP2K1 | chr15 | 66386837 | 66491544 | + |
| MAP2K2 | chr19 | 4090321 | 4124122 | − |
| MAP2K4 | chr17 | 12020829 | 12143830 | + |
| MAP3K1 | chr5 | 56815549 | 56896152 | + |
| MAP3K13 | chr3 | 185282941 | 185489094 | + |
| MAP3K14 | chr17 | 45263119 | 45317029 | − |
| MAP3K6 | chr1 | 27354067 | 27366961 | − |
| MAP3K7 | chr6 | 90513573 | 90587072 | − |
| MAPK1 | chr22 | 21759657 | 21867680 | − |
| MAPK3 | chr16 | 30114105 | 30123506 | − |
| MAPK8 | chr10 | 48306639 | 48439360 | + |
| MAPKAP1 | chr9 | 125437393 | 125707234 | − |
| MARK1 | chr1 | 220528136 | 220664461 | + |
| MARK4 | chr19 | 45079288 | 45305284 | + |
| MAST1 | chr19 | 12833951 | 12874952 | + |
| MAST2 | chr1 | 45786987 | 46036122 | + |
| MAX | chr14 | 65006174 | 65102695 | − |
| MB21D2 | chr3 | 192796815 | 192917856 | − |
| MBD1 | chr18 | 50266882 | 50281774 | − |
| MBD6 | chr12 | 57520710 | 57530148 | + |
| MBTD1 | chr17 | 51177425 | 51260163 | − |
| MCL1 | chr1 | 150560895 | 150579738 | − |
| MDC1 | chr6 | 30699807 | 30717447 | − |
| MDM2 | chr12 | 68808177 | 68845544 | + |
| MDM4 | chr1 | 204516379 | 204558120 | + |
| MDS2 | chr1 | 23581495 | 23640568 | + |
| MEAF6 | chr1 | 37489993 | 37514766 | − |
| MECOM | chr3 | 169083499 | 169663775 | − |
| MED12 | chrX | 71118556 | 71142454 | + |
| MEF2B | chr19 | 19145567 | 19192131 | − |
| MEF2C | chr5 | 88717117 | 88904257 | − |
| MEF2D | chr1 | 156463727 | 156500779 | − |
| MEN1 | chr11 | 64803510 | 64811294 | − |
| MERTK | chr2 | 111898607 | 112029561 | + |
| MET | chr7 | 116672196 | 116798377 | + |
| METAP1D | chr2 | 171999943 | 172082430 | + |
| MGA | chr15 | 41621134 | 41773081 | + |
| MGAM | chr7 | 141907813 | 142106747 | + |
| MGMT | chr10 | 129467190 | 129770983 | + |
| MIB1 | chr18 | 21704957 | 21870953 | + |
| MIDEAS | chr14 | 73715122 | 73790285 | − |
| MIR663AHG | chr20 | 26167817 | 26251546 | − |
| MITF | chr3 | 69739464 | 69968336 | + |
| MKI67 | chr10 | 128096659 | 128126423 | − |
| MKNK1 | chr1 | 46557407 | 46616843 | − |
| MLF1 | chr3 | 158571163 | 158607252 | + |
| MLH1 | chr3 | 36993350 | 37050846 | + |
| MLH3 | chr14 | 75013769 | 75051532 | − |
| MLLT1 | chr19 | 6210381 | 6279975 | − |
| MLLT10 | chr10 | 21524646 | 21743630 | + |
| MLLT11 | chr1 | 151060397 | 151069544 | + |
| MLLT3 | chr9 | 20341669 | 20622499 | − |
| MLLT6 | chr17 | 38705273 | 38729795 | + |
| MME | chr3 | 155024124 | 155183704 | + |
| MMP2 | chr16 | 55389700 | 55506691 | + |
| MN1 | chr22 | 27748277 | 27801756 | − |
| MNX1 | chr7 | 156994051 | 157010663 | − |
| MOB3B | chr9 | 27325209 | 27529814 | − |
| MPEG1 | chr11 | 59208510 | 59212927 | − |
| MPL | chr1 | 43337818 | 43354466 | + |
| MPV17L2 | chr19 | 18193218 | 18196948 | + |
| MRE11 | chr11 | 94415570 | 94493885 | − |
| MRTFA | chr22 | 40410281 | 40636719 | − |
| MRTFB | chr16 | 14071319 | 14266773 | + |
| MSH2 | chr2 | 47403067 | 47663146 | + |
| MSH3 | chr5 | 80654652 | 80876815 | + |
| MSH6 | chr2 | 47695530 | 47810063 | + |
| MSI1 | chr12 | 120341330 | 120369164 | − |
| MSI2 | chr17 | 57255851 | 57684689 | + |
| MSMB | chr10 | 46033307 | 46048180 | − |
| MSN | chrX | 65588377 | 65741931 | + |
| MST1 | chr3 | 49683947 | 49689501 | − |
| MST1R | chr3 | 49887002 | 49903873 | − |
| MTAP | chr9 | 21802636 | 21937651 | + |
| MTCP1 | chrX | 155064034 | 155147937 | − |
| MTOR | chr1 | 11106535 | 11262551 | − |
| MTR | chr1 | 236795260 | 236921278 | + |
| MTRR | chr5 | 7851186 | 7906025 | + |
| MUC1 | chr1 | 155185824 | 155192916 | − |
| MUC16 | chr19 | 8848844 | 8981342 | − |
| MUC4 | chr3 | 195746765 | 195811973 | − |
| MUSK | chr9 | 110668779 | 110806558 | + |
| MUTYH | chr1 | 45329163 | 45340893 | − |
| MYB | chr6 | 135181308 | 135219173 | + |
| MYBL1 | chr8 | 66562175 | 66614247 | − |
| MYC | chr8 | 127735434 | 127742951 | + |
| MYCL | chr1 | 39895426 | 39902256 | − |
| MYCN | chr2 | 15940550 | 15947007 | + |
| MYD88 | chr3 | 38138478 | 38143022 | + |
| MYH11 | chr16 | 15703135 | 15857028 | − |
| MYH9 | chr22 | 36281280 | 36387967 | − |
| MYO18A | chr17 | 29071122 | 29180398 | − |
| MYO19 | chr17 | 36495636 | 36543435 | − |
| MYO5A | chr15 | 52307283 | 52529050 | − |
| MYOD1 | chr11 | 17719571 | 17722136 | + |
| N4BP2 | chr4 | 40056850 | 40158252 | + |
| NAB2 | chr12 | 57089043 | 57095476 | + |
| NACA | chr12 | 56712305 | 56731628 | − |
| NADK | chr1 | 1751232 | 1780457 | − |
| NBEA | chr13 | 34942270 | 35673022 | + |
| NBEAP1 | chr15 | 20657638 | 20688408 | − |
| NBN | chr8 | 89933331 | 90003228 | − |
| NCKIPSD | chr3 | 48673844 | 48686364 | − |
| NCOA1 | chr2 | 24491254 | 24770702 | + |
| NCOA2 | chr8 | 70109782 | 70403808 | − |
| NCOA3 | chr20 | 47501887 | 47656877 | + |
| NCOA4 | chr10 | 46005088 | 46030623 | − |
| NCOR1 | chr17 | 16029157 | 16218185 | − |
| NCOR2 | chr12 | 124324415 | 124567589 | − |
| NCSTN | chr1 | 160343316 | 160358952 | + |
| NDRG1 | chr8 | 133237175 | 133302022 | − |
| NDUFS7 | chr19 | 1383527 | 1395589 | + |
| NEGR1 | chr1 | 71395943 | 72282539 | − |
| NEK6 | chr9 | 124257606 | 124353307 | + |
| NF1 | chr17 | 31094927 | 31382116 | + |
| NF2 | chr22 | 29603556 | 29698598 | + |
| NFATC2 | chr20 | 51386957 | 51562831 | − |
| NFE2 | chr12 | 54292111 | 54301015 | − |
| NFE2L2 | chr2 | 177227595 | 177392697 | − |
| NFIB | chr9 | 14081843 | 14398983 | − |
| NFKB1 | chr4 | 102501331 | 102617302 | + |
| NFKB2 | chr10 | 102394110 | 102402524 | + |
| NFKBIA | chr14 | 35401513 | 35404749 | − |
| NFKBIE | chr6 | 44258166 | 44265788 | − |
| NIN | chr14 | 50719763 | 50831162 | − |
| NKX2-1 | chr14 | 36516392 | 36521149 | − |
| NKX3-1 | chr8 | 23678697 | 23682938 | − |
| NLRP1 | chr17 | 5499427 | 5619424 | − |
| NME1 | chr17 | 51153559 | 51162428 | + |
| NOD1 | chr7 | 30424527 | 30478784 | − |
| NONO | chrX | 71254814 | 71301522 | + |
| NOTCH1 | chr9 | 136494433 | 136546048 | − |
| NOTCH2 | chr1 | 119911553 | 120100779 | − |
| NOTCH3 | chr19 | 15159038 | 15200995 | − |
| NOTCH4 | chr6 | 32194843 | 32224067 | − |
| NPM1 | chr5 | 171387116 | 171411810 | + |
| NR4A3 | chr9 | 99821855 | 99866891 | + |
| NRAS | chr1 | 114704469 | 114716771 | − |
| NRG1 | chr8 | 31639222 | 32855666 | + |
| NSD1 | chr5 | 177133025 | 177300213 | + |
| NSD2 | chr4 | 1871393 | 1982207 | + |
| NSD3 | chr8 | 38269704 | 38382272 | − |
| NT5C2 | chr10 | 103087185 | 103277605 | − |
| NTHL1 | chr16 | 2039815 | 2047866 | − |
| NTRK1 | chr1 | 156815640 | 156881850 | + |
| NTRK2 | chr9 | 84668551 | 85027054 | + |
| NTRK3 | chr15 | 87859751 | 88256768 | − |
| NUF2 | chr1 | 163266576 | 163355764 | + |
| NUMA1 | chr11 | 72002864 | 72080693 | − |
| NUMBL | chr19 | 40665905 | 40690972 | − |
| NUP214 | chr9 | 131125586 | 131234663 | + |
| NUP93 | chr16 | 56730118 | 56850286 | + |
| NUP98 | chr11 | 3671083 | 3797792 | − |
| NUTM1 | chr15 | 34343315 | 34357737 | + |
| NUTM2A | chr10 | 87225448 | 87236908 | + |
| NUTM2B | chr10 | 79703227 | 79714681 | + |
| NUTM2D | chr10 | 87357720 | 87370695 | + |
| NXF1 | chr11 | 62792123 | 62806302 | − |
| OGA | chr10 | 101784443 | 101818465 | − |
| OLIG2 | chr21 | 33025935 | 33029196 | + |
| OMD | chr9 | 92412380 | 92424471 | − |
| P2RY8 | chrX | 1462581 | 1537185 | − |
| PABPC1 | chr8 | 100685816 | 100722809 | − |
| PAFAH1B2 | chr11 | 117144284 | 117176894 | + |
| PAG1 | chr8 | 80967810 | 81112068 | − |
| PAICS | chr4 | 56435741 | 56464578 | + |
| PAK1 | chr11 | 77322017 | 77474635 | − |
| PAK3 | chrX | 110944285 | 111227361 | + |
| PAK5 | chr20 | 9537370 | 9839076 | − |
| PALB2 | chr16 | 23603160 | 23641310 | − |
| PARP1 | chr1 | 226360210 | 226408154 | − |
| PARP2 | chr14 | 20343615 | 20357904 | + |
| PARP3 | chr3 | 51942345 | 51948867 | + |
| PASK | chr2 | 241106099 | 241150264 | − |
| PATZ1 | chr22 | 31325804 | 31346346 | − |
| PAX3 | chr2 | 222199887 | 222298998 | − |
| PAX5 | chr9 | 36833269 | 37034268 | − |
| PAX7 | chr1 | 18630846 | 18748866 | + |
| PAX8 | chr2 | 113215997 | 113278921 | − |
| PBRM1 | chr3 | 52545352 | 52685917 | − |
| PBX1 | chr1 | 164555584 | 164899296 | + |
| PC | chr11 | 66848417 | 66958386 | − |
| PCBP1 | chr2 | 70087477 | 70089203 | + |
| PCDHAC2 | chr5 | 140966470 | 141012347 | + |
| PCLAF | chr15 | 64364304 | 64387687 | − |
| PCLO | chr7 | 82754012 | 83162930 | − |
| PCM1 | chr8 | 17922840 | 18027975 | + |
| PCSK7 | chr11 | 117204337 | 117232525 | − |
| PDCD1 | chr2 | 241849884 | 241858894 | − |
| PDCD11 | chr10 | 103396626 | 103446294 | + |
| PDCD1LG2 | chr9 | 5510531 | 5571282 | + |
| PDE4DIP | chr1 | 148808181 | 149048286 | + |
| PDGFB | chr22 | 39223359 | 39244982 | − |
| PDGFD | chr11 | 103907189 | 104164379 | − |
| PDGFRA | chr4 | 54229280 | 54298245 | + |
| PDGFRB | chr5 | 150113839 | 150155872 | − |
| PDK1 | chr2 | 172555373 | 172608669 | + |
| PDPK1 | chr16 | 2537979 | 2603188 | + |
| PDS5B | chr13 | 32586452 | 32778019 | + |
| PER1 | chr17 | 8140472 | 8156506 | − |
| PGAP3 | chr17 | 39671122 | 39696797 | − |
| PGBD5 | chr1 | 230314490 | 230426332 | − |
| PGLS | chr19 | 17511636 | 17521288 | + |
| PGR | chr11 | 101029624 | 101129813 | − |
| PHF1 | chr6 | 33410399 | 33416453 | + |
| PHF6 | chrX | 134373312 | 134428791 | + |
| PHKB | chr16 | 47461123 | 47701523 | + |
| PHLPP2 | chr16 | 71637835 | 71724701 | − |
| PHOX2B | chr4 | 41744082 | 41748725 | − |
| PICALM | chr11 | 85957175 | 86069882 | − |
| PIGA | chrX | 15319452 | 15335554 | − |
| PIK3C2B | chr1 | 204422628 | 204494805 | − |
| PIK3C2G | chr12 | 18242961 | 18648416 | + |
| PIK3C3 | chr18 | 41955234 | 42087830 | + |
| PIK3CA | chr3 | 179148114 | 179240093 | + |
| PIK3CB | chr3 | 138652698 | 138834928 | − |
| PIK3CD | chr1 | 9651731 | 9729114 | + |
| PIK3CG | chr7 | 106865278 | 106908980 | + |
| PIK3R1 | chr5 | 68215756 | 68301821 | + |
| PIK3R2 | chr19 | 18153163 | 18170532 | + |
| PIK3R3 | chr1 | 46040140 | 46133036 | − |
| PIM1 | chr6 | 37170152 | 37175428 | + |
| PIM2 | chrX | 48913182 | 48919024 | − |
| PKD1L2 | chr16 | 81100875 | 81220370 | − |
| PKHD1 | chr6 | 51615299 | 52087613 | − |
| PKN1 | chr19 | 14433053 | 14471867 | + |
| PLAG1 | chr8 | 56160909 | 56211324 | − |
| PLCG1 | chr20 | 41136960 | 41196801 | + |
| PLCG2 | chr16 | 81739097 | 81962685 | + |
| PLEKHG5 | chr1 | 6467122 | 6520074 | − |
| PLEKHG6 | chr12 | 6310436 | 6328506 | + |
| PLEKHS1 | chr10 | 113751262 | 113783429 | + |
| PLK2 | chr5 | 58453982 | 58460139 | − |
| PMAIP1 | chr18 | 59899996 | 59904305 | + |
| PML | chr15 | 73994673 | 74047827 | + |
| PMS1 | chr2 | 189784085 | 189877629 | + |
| PMS2 | chr7 | 5970925 | 6009106 | − |
| PNRC1 | chr6 | 89080751 | 89085160 | + |
| POLD1 | chr19 | 50384204 | 50418018 | + |
| POLE | chr12 | 132623753 | 132687376 | − |
| POLG | chr15 | 89305198 | 89334861 | − |
| POLQ | chr3 | 121431431 | 121545988 | − |
| POT1 | chr7 | 124822386 | 124929983 | − |
| POU2AF1 | chr11 | 111352255 | 111455630 | − |
| POU5F1 | chr6 | 31164337 | 31180731 | − |
| PPARG | chr3 | 12287368 | 12434356 | + |
| PPAT | chr4 | 56393362 | 56435615 | − |
| PPFIBP1 | chr12 | 27523431 | 27695564 | + |
| PPM1D | chr17 | 60600183 | 60666280 | + |
| PPP1CB | chr2 | 28751640 | 28802940 | + |
| PPP2R1A | chr19 | 52190048 | 52229518 | + |
| PPP2R2A | chr8 | 26291508 | 26372680 | + |
| PPP4R2 | chr3 | 72996803 | 73069198 | + |
| PPP6C | chr9 | 125146573 | 125189939 | − |
| PRCC | chr1 | 156750610 | 156800815 | + |
| PRDM1 | chr6 | 105993463 | 106109939 | + |
| PRDM10 | chr11 | 129899706 | 130002835 | − |
| PRDM14 | chr8 | 70051651 | 70071693 | − |
| PRDM16 | chr1 | 3069168 | 3438621 | + |
| PRDM2 | chr1 | 13700198 | 13825079 | + |
| PREX2 | chr8 | 67952046 | 68237032 | + |
| PRF1 | chr10 | 70597348 | 70602759 | − |
| PRIM1 | chr12 | 56731296 | 56752374 | − |
| PRKACA | chr19 | 14091688 | 14118084 | − |
| PRKACB | chr1 | 84078062 | 84238498 | + |
| PRKAR1A | chr17 | 68511780 | 68551319 | + |
| PRKAR2B | chr7 | 107044705 | 107161811 | + |
| PRKCA | chr17 | 66302613 | 66810743 | + |
| PRKCB | chr16 | 23835983 | 24220611 | + |
| PRKCD | chr3 | 53156009 | 53192717 | + |
| PRKCI | chr3 | 170222424 | 170305977 | + |
| PRKD1 | chr14 | 29576479 | 30191898 | − |
| PRKD2 | chr19 | 46674275 | 46717127 | − |
| PRKD3 | chr2 | 37250502 | 37324833 | − |
| PRKDC | chr8 | 47773111 | 47960178 | − |
| PRKN | chr6 | 161347417 | 162727775 | − |
| PRPF40B | chr12 | 49568218 | 49644666 | + |
| PRPF8 | chr17 | 1650629 | 1684867 | − |
| PRRX1 | chr1 | 170662728 | 170739421 | + |
| PRSS8 | chr16 | 31131433 | 31135727 | − |
| PSIP1 | chr9 | 15464066 | 15510995 | − |
| PTCH1 | chr9 | 95442980 | 95517057 | − |
| PTEN | chr10 | 87863625 | 87971930 | + |
| PTGDR | chr14 | 52267698 | 52276724 | + |
| PTGS2 | chr1 | 186671791 | 186680922 | − |
| PTK2B | chr8 | 27311482 | 27459391 | + |
| PTK6 | chr20 | 63528001 | 63537376 | − |
| PTK7 | chr6 | 43076307 | 43161719 | + |
| PTP4A1 | chr6 | 63521746 | 63583588 | + |
| PTPN1 | chr20 | 50510321 | 50585241 | + |
| PTPN11 | chr12 | 112418351 | 112509918 | + |
| PTPN13 | chr4 | 86594315 | 86815171 | + |
| PTPN2 | chr18 | 12785478 | 12929643 | − |
| PTPN6 | chr12 | 6946468 | 6961316 | + |
| PTPRB | chr12 | 70515870 | 70637440 | − |
| PTPRC | chr1 | 198638457 | 198757476 | + |
| PTPRD | chr9 | 8314246 | 10613002 | − |
| PTPRK | chr6 | 127968785 | 128520616 | − |
| PTPRO | chr12 | 15322257 | 15602175 | + |
| PTPRS | chr19 | 5158495 | 5340803 | − |
| PTPRT | chr20 | 42072752 | 43189970 | − |
| PWWP2A | chr5 | 160061801 | 160119450 | − |
| PYCR1 | chr17 | 81932384 | 81942412 | − |
| QKI | chr6 | 163414000 | 163578592 | + |
| RAB29 | chr1 | 205767986 | 205775482 | − |
| RAB35 | chr12 | 120095099 | 120117502 | − |
| RAB5C | chr17 | 42124978 | 42155044 | − |
| RABEP1 | chr17 | 5282265 | 5386340 | + |
| RAC1 | chr7 | 6374527 | 6403967 | + |
| RAC2 | chr22 | 37225270 | 37244448 | − |
| RAD17 | chr5 | 69369293 | 69414801 | + |
| RAD21 | chr8 | 116845934 | 116874776 | − |
| RAD50 | chr5 | 132556019 | 132646349 | + |
| RAD51 | chr15 | 40694774 | 40732340 | + |
| RAD51B | chr14 | 67819779 | 68730218 | + |
| RAD51C | chr17 | 58692573 | 58735611 | + |
| RAD51D | chr17 | 35092221 | 35121522 | − |
| RAD52 | chr12 | 911736 | 990053 | − |
| RAD54L | chr1 | 46246461 | 46278480 | + |
| RAF1 | chr3 | 12583601 | 12664125 | − |
| RAG1 | chr11 | 36510709 | 36593156 | + |
| RAG2 | chr11 | 36575574 | 36598279 | − |
| RALGDS | chr9 | 133097720 | 133149334 | − |
| RANBP1 | chr22 | 20115938 | 20127355 | + |
| RANBP2 | chr2 | 108719482 | 108785809 | + |
| RAP1GDS1 | chr4 | 98261384 | 98443858 | + |
| RARA | chr17 | 40309180 | 40357643 | + |
| RASA1 | chr5 | 87267883 | 87391931 | + |
| RASGEF1A | chr10 | 43194535 | 43267065 | − |
| RB1 | chr13 | 48303744 | 48599436 | + |
| RBBP6 | chr16 | 24537693 | 24572863 | + |
| RBM10 | chrX | 47145221 | 47186813 | + |
| RBM15 | chr1 | 110338506 | 110346681 | + |
| RECQL | chr12 | 21468910 | 21501669 | − |
| RECQL4 | chr8 | 144511288 | 144517845 | − |
| REL | chr2 | 60881521 | 60931612 | + |
| RELA | chr11 | 65653597 | 65663090 | − |
| RELN | chr7 | 103471381 | 103989658 | − |
| REST | chr4 | 56907876 | 56966808 | + |
| RET | chr10 | 43077064 | 43130351 | + |
| RFWD3 | chr16 | 74621399 | 74666877 | − |
| RGPD3 | chr2 | 106391290 | 106468413 | − |
| RGS7 | chr1 | 240775514 | 241357230 | − |
| RHEB | chr7 | 151466012 | 151520120 | − |
| RHOA | chr3 | 49359139 | 49412998 | − |
| RHOH | chr4 | 40191053 | 40246967 | + |
| RICTOR | chr5 | 38937920 | 39074399 | − |
| RIT1 | chr1 | 155897808 | 155911404 | − |
| RMI2 | chr16 | 11249619 | 11381662 | + |
| RNASEL | chr1 | 182573634 | 182589256 | − |
| RNF2 | chr1 | 185045526 | 185102603 | + |
| RNF213 | chr17 | 80260866 | 80398786 | + |
| RNF217-AS1 | chr6 | 124644434 | 124963165 | − |
| RNF43 | chr17 | 58353676 | 58417595 | − |
| ROBO1 | chr3 | 78597239 | 79767998 | − |
| ROBO2 | chr3 | 75906695 | 77649964 | + |
| ROS1 | chr6 | 117287353 | 117425942 | − |
| RPL10 | chrX | 154389955 | 154409168 | + |
| RPL22 | chr1 | 6185020 | 6209389 | − |
| RPL5 | chr1 | 92832013 | 92841924 | + |
| RPN1 | chr3 | 128619969 | 128681075 | − |
| RPS14 | chr5 | 150442635 | 150449739 | − |
| RPS15 | chr19 | 1438358 | 1440495 | + |
| RPS6KA2 | chr6 | 166409364 | 166906451 | − |
| RPS6KA4 | chr11 | 64359148 | 64372215 | + |
| RPS6KB2 | chr11 | 67428460 | 67435401 | + |
| RPS7 | chr2 | 3575260 | 3580920 | + |
| RPTOR | chr17 | 80544819 | 80966371 | + |
| RRAGC | chr1 | 38838198 | 38859772 | − |
| RRAS | chr19 | 49635292 | 49640143 | − |
| RRAS2 | chr11 | 14277922 | 14364506 | − |
| RRM1 | chr11 | 4094707 | 4138932 | + |
| RSPO2 | chr8 | 107899316 | 108083642 | − |
| RSPO3 | chr6 | 127118671 | 127199481 | + |
| RTEL1 | chr20 | 63657810 | 63696253 | + |
| RUNX1 | chr21 | 34787801 | 36004667 | − |
| RUNX1T1 | chr8 | 91954967 | 92103286 | − |
| RUNX2 | chr6 | 45328157 | 45664349 | + |
| RXRA | chr9 | 134317098 | 134440585 | + |
| RYBP | chr3 | 72371825 | 72446623 | − |
| S100A7 | chr1 | 153457744 | 153460651 | − |
| S1PR2 | chr19 | 10221433 | 10231331 | − |
| SALL4 | chr20 | 51782331 | 51802521 | − |
| SAMD9 | chr7 | 93099513 | 93118023 | − |
| SAMHD1 | chr20 | 36890229 | 36951893 | − |
| SBDS | chr7 | 66987680 | 66995587 | − |
| SCG5 | chr15 | 32641676 | 32697098 | + |
| SDC4 | chr20 | 45325288 | 45348424 | − |
| SDHA | chr5 | 218241 | 257082 | + |
| SDHAF2 | chr11 | 61430042 | 61446733 | + |
| SDHB | chr1 | 17018722 | 17054032 | − |
| SDHC | chr1 | 161314381 | 161363206 | + |
| SDHD | chr11 | 112086824 | 112120016 | + |
| SEC31A | chr4 | 82818509 | 82901166 | − |
| SEMA6A | chr5 | 116443555 | 116574823 | − |
| SEPTIN5 | chr22 | 19714503 | 19724224 | + |
| SEPTIN6 | chrX | 119615724 | 119693370 | − |
| SEPTIN9 | chr17 | 77280569 | 77500596 | + |
| SERP2 | chr13 | 44373665 | 44397714 | + |
| SERPINA9 | chr14 | 94462717 | 94479689 | − |
| SERPINB3 | chr18 | 63655197 | 63661893 | − |
| SERPINB4 | chr18 | 63637259 | 63644256 | − |
| SESN1 | chr6 | 108986437 | 109094819 | − |
| SESN2 | chr1 | 28259518 | 28282491 | + |
| SESN3 | chr11 | 95165513 | 95232541 | − |
| SET | chr9 | 128683424 | 128696400 | + |
| SETBP1 | chr18 | 44680173 | 45068510 | + |
| SETD1A | chr16 | 30957754 | 30984664 | + |
| SETD1B | chr12 | 121804009 | 121832656 | + |
| SETD2 | chr3 | 47016429 | 47164113 | − |
| SETD3 | chr14 | 99397748 | 99480889 | − |
| SETD4 | chr21 | 36034541 | 36079389 | − |
| SETD5 | chr3 | 9397615 | 9479240 | + |
| SETD6 | chr16 | 58515479 | 58523842 | + |
| SETD7 | chr4 | 139495941 | 139606699 | − |
| SETDB1 | chr1 | 150926263 | 150964744 | + |
| SETDB2 | chr13 | 49444374 | 49495003 | + |
| SF3B1 | chr2 | 197388515 | 197435079 | − |
| SFPQ | chr1 | 35176378 | 35193145 | − |
| SFRP1 | chr8 | 41261962 | 41309473 | − |
| SFRP2 | chr4 | 153780591 | 153789083 | − |
| SFRP4 | chr7 | 37905932 | 38025695 | − |
| SGK1 | chr6 | 134169248 | 134318112 | − |
| SH2B3 | chr12 | 111405923 | 111451623 | + |
| SH2D1A | chrX | 124227868 | 124373197 | + |
| SH3BP5 | chr3 | 15254353 | 15341368 | − |
| SH3GL1 | chr19 | 4360370 | 4400547 | − |
| SHOC2 | chr10 | 110919593 | 111013666 | + |
| SHQ1 | chr3 | 72749277 | 72861914 | − |
| SHTN1 | chr10 | 116881477 | 117126586 | − |
| SIRPA | chr20 | 1894167 | 1940592 | + |
| SIX1 | chr14 | 60643421 | 60658259 | − |
| SIX2 | chr2 | 45005182 | 45009452 | − |
| SKI | chr1 | 2228319 | 2310213 | + |
| SLC1A2 | chr11 | 35251205 | 35420063 | − |
| SLC29A1 | chr6 | 44219553 | 44234142 | + |
| SLC34A2 | chr4 | 25648011 | 25678748 | + |
| SLC45A3 | chr1 | 205657851 | 205680509 | − |
| SLFN11 | chr17 | 35350305 | 35373701 | − |
| SLX4 | chr16 | 3581181 | 3611606 | − |
| SMAD2 | chr18 | 47808957 | 47931146 | − |
| SMAD3 | chr15 | 67063763 | 67195173 | + |
| SMAD4 | chr18 | 51028394 | 51085045 | + |
| SMARCA1 | chrX | 129446501 | 129523500 | − |
| SMARCA2 | chr9 | 1980290 | 2193624 | + |
| SMARCA4 | chr19 | 10960932 | 11079426 | + |
| SMARCB1 | chr22 | 23786931 | 23838009 | + |
| SMARCD1 | chr12 | 50085200 | 50100707 | + |
| SMARCE1 | chr17 | 40624962 | 40648654 | − |
| SMC1A | chrX | 53374149 | 53422728 | − |
| SMC3 | chr10 | 110567695 | 110606048 | + |
| SMG1 | chr16 | 18804860 | 18926408 | − |
| SMO | chr7 | 129188633 | 129213545 | + |
| SMUG1 | chr12 | 54121277 | 54189008 | − |
| SMYD3 | chr1 | 245749342 | 246507312 | − |
| SNCAIP | chr5 | 122311354 | 122464219 | + |
| SND1 | chr7 | 127652194 | 128092609 | + |
| SNX29 | chr16 | 11976734 | 12574287 | + |
| SNX31 | chr8 | 100572889 | 100663415 | − |
| SOCS1 | chr16 | 11254417 | 11256204 | − |
| SOCS2 | chr12 | 93569814 | 93583487 | + |
| SOCS3 | chr17 | 78356778 | 78360077 | − |
| SOS1 | chr2 | 38981549 | 39124345 | − |
| SOX10 | chr22 | 37970686 | 37987422 | − |
| SOX11 | chr2 | 5692384 | 5701385 | + |
| SOX17 | chr8 | 54457935 | 54460892 | + |
| SOX2 | chr3 | 181711925 | 181714436 | + |
| SOX21 | chr13 | 94709622 | 94712545 | − |
| SOX9 | chr17 | 72121020 | 72126416 | + |
| SP140 | chr2 | 230203110 | 230313215 | + |
| SPECC1 | chr17 | 20008865 | 20319026 | + |
| SPEN | chr1 | 15836095 | 15940456 | + |
| SPI1 | chr11 | 47354860 | 47378547 | − |
| SPOP | chr17 | 49598884 | 49678163 | − |
| SPRED1 | chr15 | 38252836 | 38357249 | + |
| SPRTN | chr1 | 231337104 | 231355023 | + |
| SPTA1 | chr1 | 158610704 | 158686715 | − |
| SRC | chr20 | 37344685 | 37406050 | + |
| SRGAP3 | chr3 | 8980591 | 9363053 | − |
| SRSF2 | chr17 | 76734115 | 76737333 | − |
| SRSF3 | chr6 | 36594353 | 36605600 | + |
| SS18 | chr18 | 26016253 | 26091217 | − |
| SS18L1 | chr20 | 62143769 | 62182514 | + |
| SSH1 | chr12 | 108778191 | 108857590 | − |
| SSR3 | chr3 | 156539553 | 156555149 | − |
| SSX1 | chrX | 48255392 | 48267444 | + |
| SSX2 | chrX | 52696896 | 52707189 | − |
| SSX4 | chrX | 48383516 | 48393347 | + |
| STAG1 | chr3 | 136336236 | 136752403 | − |
| STAG2 | chrX | 123960212 | 124422664 | + |
| STAT1 | chr2 | 190908460 | 191020960 | − |
| STAT3 | chr17 | 42313324 | 42388540 | − |
| STAT4 | chr2 | 191029576 | 191151596 | − |
| STAT5A | chr17 | 42287547 | 42311943 | + |
| STAT5B | chr17 | 42199177 | 42276707 | − |
| STAT6 | chr12 | 57095408 | 57132139 | − |
| STIL | chr1 | 47250139 | 47314892 | − |
| STK11 | chr19 | 1177558 | 1228431 | + |
| STK19 | chr6 | 31971091 | 31982821 | + |
| STK36 | chr2 | 218672069 | 218702716 | + |
| STK40 | chr1 | 36339624 | 36385924 | − |
| STRBP | chr9 | 123109500 | 123268586 | − |
| STRN | chr2 | 36837698 | 36966536 | − |
| STX11 | chr6 | 144150517 | 144191939 | + |
| STX7 | chr6 | 132445867 | 132513198 | − |
| SUFU | chr10 | 102503972 | 102633535 | + |
| SUZ12 | chr17 | 31937007 | 32001038 | + |
| SYK | chr9 | 90801787 | 90898549 | + |
| SYNE1 | chr6 | 152121687 | 152637801 | − |
| SYT1 | chr12 | 78863993 | 79452008 | + |
| TAF1 | chrX | 71366222 | 71532374 | + |
| TAF15 | chr17 | 35713791 | 35864615 | + |
| TAF1L | chr9 | 32629454 | 32635669 | − |
| TAL1 | chr1 | 47216290 | 47232220 | − |
| TAL2 | chr9 | 105662457 | 105663124 | + |
| TAP1 | chr6 | 32845209 | 32853816 | − |
| TAP2 | chr6 | 32821833 | 32838770 | − |
| TBL1XR1 | chr3 | 177019340 | 177228000 | − |
| TBX22 | chrX | 80014753 | 80031774 | + |
| TBX3 | chr12 | 114670255 | 114684175 | − |
| TCEA1 | chr8 | 53966552 | 54022456 | − |
| TCF12 | chr15 | 56918623 | 57299281 | + |
| TCF3 | chr19 | 1609290 | 1652615 | − |
| TCF7L1 | chr2 | 85133392 | 85310387 | + |
| TCF7L2 | chr10 | 112950247 | 113167678 | + |
| TCL1A | chr14 | 95709947 | 95714196 | − |
| TCL1B | chr14 | 95686426 | 95692628 | + |
| TEC | chr4 | 48135783 | 48269838 | − |
| TEK | chr9 | 27109141 | 27230174 | + |
| TEKT4 | chr2 | 94871430 | 94876823 | + |
| TENT5C | chr1 | 117606048 | 117628389 | + |
| TERC | chr3 | 169764520 | 169765060 | − |
| TERT | chr5 | 1253147 | 1295068 | − |
| TET1 | chr10 | 68560337 | 68694487 | + |
| TET2 | chr4 | 105145875 | 105279816 | + |
| TET3 | chr2 | 73984910 | 74108176 | + |
| TFE3 | chrX | 49028726 | 49043410 | − |
| TFEB | chr6 | 41683978 | 41736259 | − |
| TFG | chr3 | 100709295 | 100748964 | + |
| TFPT | chr19 | 54107020 | 54115657 | − |
| TFRC | chr3 | 196027183 | 196082096 | − |
| TGFB1 | chr19 | 41301587 | 41353922 | − |
| TGFBR1 | chr9 | 99104038 | 99154192 | + |
| TGFBR2 | chr3 | 30606601 | 30694142 | + |
| TGFBR3 | chr1 | 91680343 | 91906335 | − |
| TGM7 | chr15 | 43276271 | 43302255 | − |
| THADA | chr2 | 43230851 | 43596038 | − |
| THBS1 | chr15 | 39581079 | 39599466 | + |
| THRAP3 | chr1 | 36224432 | 36305357 | + |
| TIMP3 | chr22 | 32801705 | 32863041 | + |
| TIPARP | chr3 | 156673235 | 156706770 | + |
| TLL2 | chr10 | 96364608 | 96513926 | − |
| TLR2 | chr4 | 153684070 | 153706260 | + |
| TLR4 | chr9 | 117704175 | 117724735 | + |
| TLX1 | chr10 | 101131300 | 101137789 | + |
| TLX3 | chr5 | 171309248 | 171312139 | + |
| TMEM127 | chr2 | 96248514 | 96265997 | − |
| TMEM216 | chr11 | 61392360 | 61398863 | + |
| TMEM30A | chr6 | 75252924 | 75284948 | − |
| TMPRSS2 | chr21 | 41464300 | 41531116 | − |
| TMSB4X | chrX | 12975110 | 12977227 | + |
| TMSB4XP8 | chr4 | 90838903 | 90839037 | − |
| TNC | chr9 | 115019575 | 115118257 | − |
| TNFAIP3 | chr6 | 137867214 | 137883312 | + |
| TNFRSF11A | chr18 | 62325287 | 62391288 | + |
| TNFRSF13B | chr17 | 16929816 | 16972118 | − |
| TNFRSF14 | chr1 | 2555639 | 2565382 | + |
| TNFRSF17 | chr16 | 11965210 | 11968068 | + |
| TNFRSF1B | chr1 | 12166991 | 12209228 | + |
| TNFSF4 | chr1 | 173183731 | 173207331 | − |
| TNK2 | chr3 | 195863364 | 195911945 | − |
| TOP1 | chr20 | 41028822 | 41124487 | + |
| TP53 | chr17 | 7661779 | 7687538 | − |
| TP53BP1 | chr15 | 43403061 | 43510728 | − |
| TP63 | chr3 | 189631389 | 189897276 | + |
| TPM3 | chr1 | 154155304 | 154194648 | − |
| TPM4 | chr19 | 16067021 | 16103002 | + |
| TPR | chr1 | 186311652 | 186375693 | − |
| TRAF2 | chr9 | 136881912 | 136926607 | + |
| TRAF3 | chr14 | 102777449 | 102911500 | + |
| TRAF5 | chr1 | 211326615 | 211374946 | + |
| TRAF7 | chr16 | 2155698 | 2178129 | + |
| TRHDE | chr12 | 72087266 | 72670758 | + |
| TRIM24 | chr7 | 138460259 | 138589996 | + |
| TRIM27 | chr6 | 28903002 | 28923988 | − |
| TRIM33 | chr1 | 114392790 | 114511203 | − |
| TRIP11 | chr14 | 91965991 | 92040896 | − |
| TRIP13 | chr5 | 892884 | 919357 | + |
| TRRAP | chr7 | 98877933 | 99050831 | + |
| TSC1 | chr9 | 132891348 | 132946874 | − |
| TSC2 | chr16 | 2047967 | 2089491 | + |
| TSHR | chr14 | 80954989 | 81146306 | + |
| TSPAN31 | chr12 | 57738013 | 57750219 | + |
| TTL | chr2 | 112482156 | 112541739 | + |
| TUSC3 | chr8 | 15417215 | 15766649 | + |
| TYK2 | chr19 | 10350529 | 10380572 | − |
| TYRO3 | chr15 | 41557675 | 41583589 | + |
| U2AF1 | chr21 | 43092956 | 43107570 | − |
| U2AF2 | chr19 | 55654146 | 55674716 | + |
| UBR5 | chr8 | 102252273 | 102412759 | − |
| UEVLD | chr11 | 18529609 | 18588747 | − |
| UGT1A1 | chr2 | 233760270 | 233773300 | + |
| UMODL1 | chr21 | 42062959 | 42143453 | + |
| UNC13A | chr19 | 17601336 | 17688365 | − |
| UPF1 | chr19 | 18831959 | 18868230 | + |
| USP44 | chr12 | 95516560 | 95551476 | − |
| USP6 | chr17 | 5116032 | 5175034 | + |
| USP8 | chr15 | 50424380 | 50514421 | + |
| USP9X | chrX | 41085445 | 41236579 | + |
| VAV1 | chr19 | 6772708 | 6857366 | + |
| VAV2 | chr9 | 133761894 | 133992604 | − |
| VEGFA | chr6 | 43770184 | 43786487 | + |
| VGLL2 | chr6 | 117265558 | 117273565 | + |
| VGLL3 | chr3 | 86876388 | 86991149 | − |
| VHL | chr3 | 10141778 | 10153667 | + |
| VTCN1 | chr1 | 117143587 | 117210960 | − |
| VTI1A | chr10 | 112446998 | 112818744 | + |
| WAS | chrX | 48676596 | 48691427 | + |
| WDCP | chr2 | 24029347 | 24049575 | − |
| WDR62 | chr19 | 36054649 | 36105108 | + |
| WDR90 | chr16 | 649311 | 667833 | + |
| WIF1 | chr12 | 65050626 | 65121305 | − |
| WNK2 | chr9 | 93184916 | 93320572 | + |
| WRN | chr8 | 31033788 | 31176138 | + |
| WT1 | chr11 | 32387775 | 32435564 | − |
| WWP1 | chr8 | 86342547 | 86478420 | + |
| WWTR1 | chr3 | 149517235 | 149736714 | − |
| XBP1 | chr22 | 28794555 | 28800597 | − |
| XIAP | chrX | 123859724 | 123913976 | + |
| XKR4 | chr8 | 55102028 | 55542054 | + |
| XPA | chr9 | 97674909 | 97697340 | − |
| XPC | chr3 | 14145147 | 14178621 | − |
| XPO1 | chr2 | 61476032 | 61538741 | − |
| XRCC2 | chr7 | 152644776 | 152676141 | − |
| YAF2 | chr12 | 42157104 | 42238349 | − |
| YAP1 | chr11 | 102110447 | 102233424 | + |
| YES1 | chr18 | 721588 | 812546 | − |
| YPEL5 | chr2 | 30146941 | 30160533 | + |
| YWHAE | chr17 | 1344275 | 1400222 | − |
| YY1 | chr14 | 100238298 | 100282788 | + |
| YY1AP1 | chr1 | 155659443 | 155689000 | − |
| ZBTB16 | chr11 | 114059041 | 114256765 | + |
| ZBTB20 | chr3 | 114314500 | 115147288 | − |
| ZBTB7A | chr19 | 4043303 | 4066899 | − |
| ZCCHC7 | chr9 | 37120574 | 37358149 | + |
| ZCCHC8 | chr12 | 122471599 | 122501073 | − |
| ZEB1 | chr10 | 31318495 | 31529814 | + |
| ZFHX3 | chr16 | 72782885 | 73891871 | − |
| ZFP36L1 | chr14 | 68787660 | 68796253 | − |
| ZMYM2 | chr13 | 19958677 | 20091829 | + |
| ZMYM3 | chrX | 71239624 | 71255146 | − |
| ZNF217 | chr20 | 53567071 | 53609907 | − |
| ZNF24 | chr18 | 35332227 | 35345482 | − |
| ZNF318 | chr6 | 43307134 | 43369647 | − |
| ZNF331 | chr19 | 53519527 | 53580269 | + |
| ZNF384 | chr12 | 6666477 | 6689572 | − |
| ZNF429 | chr19 | 21496682 | 21556270 | + |
| ZNF443 | chr19 | 12429706 | 12441021 | − |
| ZNF479 | chr7 | 57117676 | 57139864 | − |
| ZNF521 | chr18 | 25061924 | 25352190 | − |
| ZNF526 | chr19 | 42220312 | 42228201 | + |
| ZNF544 | chr19 | 58228594 | 58277495 | + |
| ZNF559 | chr19 | 9323772 | 9351162 | + |
| ZNF638 | chr2 | 71276561 | 71435069 | + |
| ZNF703 | chr8 | 37695782 | 37700019 | + |
| ZNF750 | chr17 | 82829434 | 82840022 | − |
| ZNRF3 | chr22 | 28883572 | 29057488 | + |
| ZRSR2 | chrX | 15790472 | 15823260 | + |
| ZSWIM4 | chr19 | 13795460 | 13832230 | + |

APPENDIX 2

| Cancer | | | | | Gene | | | Target | |
|---|---|---|---|---|---|---|---|---|---|
| gene name | Chromosome | Gene Start | Gene End | Strand | Length | Target Start | Target End | Length | Group |
| ABL1 | chr9 | 130713016 | 130887675 | + | 174,659 | 130,713,016 | 130,887,675 | 174,659 | 1 |
| ALK | chr2 | 29192774 | 29921586 | − | 728,812 | 29,192,774 | 29,921,586 | 728,812 | 1 |
| ATM | chr11 | 108223044 | 108369102 | + | 146,058 | 108,223,044 | 108,369,102 | 146,058 | 1 |
| BCR | chr22 | 23179704 | 23318037 | + | 138,333 | 23,179,704 | 23,318,037 | 138,333 | 1 |
| BRAF | chr7 | 140719327 | 140924929 | − | 205,602 | 140,719,327 | 140,924,929 | 205,602 | 1 |
| BRCA1 | chr17 | 43044295 | 43170245 | − | 125,950 | 43,044,295 | 43,170,245 | 125,950 | 1 |
| BRCA2 | chr13 | 32315086 | 32400268 | + | 85,182 | 32,307,676 | 32,407,678 | 100,002 | 1 |
| BTK | chrX | 101349447 | 101390796 | − | 41,349 | 101,320,121 | 101,420,122 | 100,001 | 1 |
| CD274 | chr9 | 5450503 | 5470566 | + | 20,063 | 5,410,534 | 5,510,535 | 100,001 | 1 |
| CHEK1 | chr11 | 125625163 | 125676255 | + | 51,092 | 125,600,708 | 125,700,710 | 100,002 | 1 |
| CHEK2 | chr22 | 28687743 | 28742422 | − | 54,679 | 28,665,082 | 28,765,083 | 100,001 | 1 |
| EGFR | chr7 | 55019017 | 55211628 | + | 192,611 | 55,019,017 | 55,211,628 | 192,611 | 1 |
| ESR1 | chr6 | 151656691 | 152129619 | + | 472,928 | 151,656,691 | 152,129,619 | 472,928 | 1 |
| ETV1 | chr7 | 13891229 | 13991425 | − | 100,196 | 13,891,229 | 13,991,425 | 100,196 | 1 |
| ETV4 | chr17 | 43527844 | 43579620 | − | 51,776 | 43,503,731 | 43,603,733 | 100,002 | 1 |
| ETV6 | chr12 | 11649674 | 11895377 | + | 245,703 | 11,649,674 | 11,895,377 | 245,703 | 1 |
| EZH2 | chr7 | 148807257 | 148884321 | − | 77,064 | 148,795,788 | 148,895,790 | 100,002 | 1 |
| FGFR1 | chr8 | 38400215 | 38468834 | − | 68,619 | 38,384,524 | 38,484,525 | 100,001 | 1 |
| FGFR2 | chr10 | 121478332 | 121598458 | − | 120,126 | 121,478,332 | 121,598,458 | 120,126 | 1 |
| FGFR3 | chr4 | 1793293 | 1808872 | + | 15,579 | 1,751,082 | 1,851,083 | 100,001 | 1 |
| FOXO1 | chr13 | 40555667 | 40666641 | − | 110,974 | 40,555,667 | 40,666,641 | 110,974 | 1 |
| HRAS | chr11 | 532242 | 537321 | − | 5,079 | 484,781 | 584,782 | 100,001 | 1 |
| IDH1 | chr2 | 208236229 | 208266074 | − | 29,845 | 208,201,151 | 208,301,152 | 100,001 | 1 |
| IDH2 | chr15 | 90083045 | 90102477 | − | 19,432 | 90,042,760 | 90,142,762 | 100,002 | 1 |
| KRAS | chr12 | 25205246 | 25250936 | − | 45,690 | 25,178,090 | 25,278,092 | 100,002 | 1 |
| MAP2K1 | chr15 | 66386837 | 66491544 | + | 104,707 | 66,386,837 | 66,491,544 | 104,707 | 1 |
| MAP2K2 | chr19 | 4090321 | 4124122 | − | 33,801 | 4,057,221 | 4,157,222 | 100,001 | 1 |
| MDM2 | chr12 | 68808177 | 68845544 | + | 37,367 | 68,776,860 | 68,876,861 | 100,001 | 1 |
| MET | chr7 | 116672196 | 11679837 | + | 126,181 | 116,672,196 | 116,798,377 | 126,181 | 1 |
| MYBL1 | chr8 | 66562175 | 66614247 | − | 52,072 | 66,538,210 | 66,638,212 | 100,002 | 1 |
| MYC | chr8 | 127735434 | 127742951 | + | 7,517 | 127,689,192 | 127,789,193 | 100,001 | 1 |
| NRAS | chr1 | 114704469 | 114716771 | − | 12,302 | 114,660,619 | 114,760,621 | 100,002 | 1 |
| NRG1 | chr8 | 31639222 | 32855666 | + | 1,216,444 | 31,639,222 | 32,855,666 | 1,216,444 | 1 |
| NTRK1 | chr1 | 156815640 | 156881850 | + | 66,210 | 156,798,744 | 156,898,746 | 100,002 | 1 |
| NTRK2 | chr9 | 84668551 | 85027054 | + | 358,503 | 84,668,551 | 85,027,054 | 358,503 | 1 |
| NTRK3 | chr15 | 87859751 | 88256768 | − | 397,017 | 87,859,751 | 88,256,768 | 397,017 | 1 |
| PDCD1 | chr2 | 241849884 | 241858894 | − | 9,010 | 241,804,388 | 241,904,390 | 100,002 | 1 |
| PDGFRA | chr4 | 54229280 | 54298245 | + | 68,965 | 54,213,762 | 54,313,763 | 100,001 | 1 |
| PDGFRB | chr5 | 150113839 | 150155872 | − | 42,033 | 150,084,855 | 150,184,856 | 100,001 | 1 |
| PIK3CA | chr3 | 179148114 | 179240093 | + | 91,979 | 179,144,103 | 179,244,104 | 100,001 | 1 |
| PLAG1 | chr8 | 56160909 | 56211324 | − | 50,415 | 56,136,116 | 56,236,117 | 100,001 | 1 |
| RAD51B | chr14 | 67819779 | 68730218 | + | 910,439 | 67,819,779 | 68,730,218 | 910,439 | 1 |
| RAD51C | chr17 | 58692573 | 58735611 | + | 43,038 | 58,664,091 | 58,764,093 | 100,002 | 1 |
| RAD51D | chr17 | 35092221 | 35121522 | − | 29,301 | 35,056,871 | 35,156,872 | 100,001 | 1 |
| RAD54L | chr1 | 46246461 | 46278480 | + | 32,019 | 46,212,470 | 46,312,471 | 100,001 | 1 |
| RET | chr10 | 43077064 | 43130351 | + | 53,287 | 43,053,707 | 43,153,708 | 100,001 | 1 |
| ROS1 | chr6 | 117287353 | 117425942 | − | 138,589 | 117,287,353 | 117,425,942 | 138,589 | 1 |
| TERT | chr5 | 1253147 | 1295068 | − | 41,921 | 1,224,107 | 1,324,108 | 100,001 | 1 |
| TIGIT | chr3 | 114276913 | 114310288 | + | 33,375 | 114,243,600 | 114,343,601 | 100,001 | 1 |
| TMPRSS2 | chr21 | 41464300 | 41531116 | − | 66,816 | 41,447,707 | 41,547,709 | 100,002 | 1 |
| AKT1 | chr14 | 104769349 | 104795751 | − | 26,402 | 104,732,549 | 104,832,551 | 100,002 | [symbol] |
| AR | chrX | 67544021 | 67730619 | + | 186,598 | 67,544,021 | 67,730,619 | 186,598 | [symbol] |
| ARAF | chrX | 47561205 | 47571908 | + | 10,703 | 47,516,556 | 47,616,557 | 100,001 | 2 |
| ATR | chr3 | 142449007 | 142578733 | − | 129,726 | 142,449,007 | 142,578,733 | 129,726 | 2 |
| AURKA | chr20 | 56369389 | 56392337 | − | 22,948 | 56,330,862 | 56,430,864 | 100,002 | 2 |
| BAP1 | chr3 | 52401008 | 52410008 | − | 9,000 | 52,355,507 | 52,455,509 | 100,002 | 2 |
| BARD1 | chr2 | 214725646 | 214809683 | − | 84,037 | 214,717,664 | 214,817,665 | 100,001 | 2 |
| BCL2 | chr18 | 63123346 | 63320128 | − | 196,782 | 63,123,346 | 63,320,128 | 196,782 | 2 |
| BCOR | chrX | 40049815 | 40177329 | − | 127,514 | 40,049,815 | 40,177,329 | 127,514 | 2 |
| BRIP1 | chr17 | 61679139 | 61863559 | − | 184,420 | 61,679,139 | 61,863,559 | 184,420 | 2 |
| CAMTA1 | chr1 | 6785454 | 7769706 | + | 984,252 | 6,785,454 | 7,769,706 | 984,252 | 2 |
| CCNB3 | chrX | 50202713 | 50351914 | + | 149,201 | 50,202,713 | 50,351,914 | 149,201 | 2 |
| CDK12 | chr17 | 39461486 | 39564907 | + | 103,421 | 39,461,486 | 39,564,907 | 103,421 | 2 |
| CDK4 | chr12 | 57747727 | 57756013 | − | 8,286 | 57,701,869 | 57,801,871 | 100,002 | 2 |
| CDK6 | chr7 | 92604921 | 92836573 | − | 231,652 | 92,604,921 | 92,836,573 | 231,652 | 2 |
| CDKN2A | chr9 | 21967752 | 21995301 | − | 27,549 | 21,931,526 | 22,031,527 | 100,001 | 2 |
| CIC | chr19 | 42268537 | 42295797 | + | 27,260 | 42,232,166 | 42,332,168 | 100,002 | [symbol] |
| CLDN18 | chr3 | 137998735 | 138033655 | + | 34,920 | 137,966,194 | 138,066,196 | 100,002 | [symbol] |
| CSF1 | chr1 | 109910242 | 109930992 | + | 20,750 | 109,870,616 | 109,970,618 | 100,002 | [symbol] |
| CSF1R | chr5 | 150053291 | 150113372 | − | 60,081 | 150,033,331 | 150,133,332 | 100,001 | [symbol] |
| EPC1 | chr10 | 32267751 | 32378798 | − | 111,047 | 32,267,751 | 32,378,798 | 111,047 | [symbol] |
| ERBB2 | chr17 | 39687914 | 39730426 | + | 42,512 | 39,659,169 | 39,759,171 | 100,002 | 2 |
| ERCC2 | chr19 | 45349837 | 45370918 | − | 21,081 | 45,310,377 | 45,410,378 | 100,001 | 2 |
| ERG | chr21 | 38380027 | 38661780 | − | 281,753 | 38,380,027 | 38,661,780 | 281,753 | 2 |
| EWSR1 | chr22 | 29268009 | 29300525 | + | 32,516 | 29,234,266 | 29,334,268 | 100,002 | 2 |
| FANCL | chr2 | 58159243 | 58241372 | − | 82,129 | 58,150,307 | 58,250,308 | 100,001 | 2 |
| FGFR4 | chr5 | 177086905 | 177098144 | + | 11,239 | 177,042,524 | 177,142,525 | 100,001 | 2 |
| FH | chr1 | 241497511 | 241519799 | − | 22,288 | 241,458,654 | 241,558,656 | 100,002 | 2 |
| FLCN | chr17 | 17212212 | 17237188 | − | 24,976 | 17,174,699 | 17,274,701 | 100,002 | 2 |
| FLI1 | chr11 | 128686535 | 128813267 | + | 126,732 | 128,686,535 | 128,813,267 | 126,732 | 2 |
| FLT3 | chr13 | 28003274 | 28100592 | − | 97,318 | 28,001,932 | 28,101,934 | 100,002 | 2 |
| FOSB | chr19 | 45467995 | 45475179 | + | 7,184 | 45,421,586 | 45,521,588 | 100,002 | 2 |
| FUS | chr16 | 31180138 | 31191605 | + | 11,467 | 31,135,871 | 31,235,872 | 100,001 | 2 |
| GLI1 | chr12 | 57459785 | 57472268 | + | 12,483 | 57,416,026 | 57,516,027 | 100,001 | 2 |
| HMGA2 | chr12 | 65824460 | 65966291 | + | 141,831 | 65,824,460 | 65,966,291 | 141,831 | 2 |
| JAK2 | chr9 | 4984390 | 5129948 | + | 145,558 | 4,984,390 | 5,129,948 | 145,558 | 2 |
| JAZF1 | chr7 | 27830573 | 28180795 | − | 350,222 | 27,830,573 | 28,180,795 | 350,222 | 2 |
| KIT | chr4 | 54657918 | 54740715 | + | 82,797 | 54,649,316 | 54,749,317 | 100,001 | 2 |
| KMT2A | chr11 | 118436490 | 118526832 | + | 90,342 | 118,431,660 | 118,531,662 | 100,002 | 2 |
| MAPK1 | chr22 | 21759657 | 21867680 | − | 108,023 | 21,759,657 | 21,867,680 | 108,023 | 2 |
| MEAF6 | chr1 | 37489993 | 37514766 | − | 24,773 | 37,452,379 | 37,552,380 | 100,001 | 2 |
| MGMT | chr10 | 129467190 | 129770983 | + | 303,793 | 129,467,190 | 129,770,983 | 303,793 | 2 |
| MLH1 | chr3 | 36993350 | 37050846 | + | 57,496 | 36,972,097 | 37,072,099 | 100,002 | 2 |
| MSH2 | chr2 | 47403067 | 47663146 | + | 260,079 | 47,403,067 | 47,663,146 | 260,079 | 2 |
| MSH6 | chr2 | 47695530 | 47810063 | + | 114,533 | 47,695,530 | 47,810,063 | 114,533 | 2 |
| MTOR | chr1 | 11106535 | 11262551 | − | 156,016 | 11,106,535 | 11,262,551 | 156,016 | 2 |
| MYB | chr6 | 135181308 | 135219173 | + | 37,865 | 135,150,240 | 135,250,241 | 100,001 | 2 |
| NCOA2 | chr8 | 70109782 | 70403808 | − | 294,026 | 70,109,782 | 70,403,808 | 294,026 | 2 |
| NCOA3 | chr20 | 47501887 | 47656877 | + | 154,990 | 47,501,887 | 47,656,877 | 154,990 | 2 |
| NF1 | chr17 | 31094927 | 31382116 | + | 287,189 | 31,094,927 | 31,382,116 | 287,189 | 2 |
| NPM1 | chr5 | 171387116 | 171411810 | + | 24,694 | 171,349,462 | 171,449,464 | 100,002 | 2 |
| NUTM1 | chr15 | 34343315 | 34357737 | + | 14,422 | 34,300,525 | 34,400,527 | 100,002 | 2 |
| NUTM2A | chr10 | 87225448 | 87236908 | + | 11,460 | 87,181,177 | 87,281,179 | 100,002 | 2 |
| PALB2 | chr16 | 23603160 | 23641310 | − | 38,150 | 23,572,234 | 23,672,236 | 100,002 | 2 |
| PAX3 | chr2 | 222199887 | 222298998 | − | 99,111 | 222,199,442 | 222,299,443 | 100,001 | 2 |
| PAX7 | chr1 | 18630846 | 18748866 | + | 118,020 | 18,630,846 | 18,748,866 | 118,020 | 2 |
| PDGFB | chr22 | 39223359 | 39244982 | − | 21,623 | 39,184,170 | 39,284,171 | 100,001 | 2 |
| PGR | chr11 | 101029624 | 101129813 | − | 100,189 | 101,029,624 | 101,129,813 | 100,189 | 2 |
| PHF1 | chr6 | 33410399 | 33416453 | + | 6,054 | 33,363,425 | 33,463,427 | 100,002 | 2 |
| PMS2 | chr7 | 5970925 | 6009106 | − | 38,181 | 5,940,015 | 6,040,016 | 100,001 | 2 |
| PTCH1 | chr9 | 95442980 | 95517057 | − | 74,077 | 95,430,018 | 95,530,019 | 100,001 | 2 |
| PTEN | chr10 | 87863625 | 87971930 | + | 108,305 | 87,863,625 | 87,971,930 | 108,305 | 2 |
| RAD51 | chr15 | 40694774 | 40732340 | + | 37,566 | 40,663,556 | 40,763,558 | 100,002 | 2 |
| RAF1 | chr3 | 12583601 | 12664125 | − | 80,524 | 12,573,862 | 12,673,864 | 100,002 | 2 |
| RELA | chr11 | 65653597 | 65663090 | − | 9,493 | 65,608,343 | 65,708,344 | 100,001 | 2 |
| SDHA | chr5 | 218241 | 257082 | + | 38,841 | 187,661 | 287,662 | 100,001 | 2 |
| SDHB | chr1 | 17018722 | 17054032 | − | 35,310 | 16,986,376 | 17,086,378 | 100,002 | 2 |
| SDHC | chr1 | 161314381 | 161363206 | + | 48,825 | 161,288,793 | 161,388,794 | 100,001 | 2 |
| SDHD | chr11 | 112086824 | 112120016 | + | 33,192 | 112,053,419 | 112,153,421 | 100,002 | 2 |
| SMARCB1 | chr22 | 23786931 | 23838009 | + | 51,078 | 23,762,469 | 23,862,471 | 100,002 | 2 |
| SS18 | chr18 | 26016253 | 26091217 | − | 74,964 | 26,003,734 | 26,103,736 | 100,002 | 2 |
| STAT6 | chr12 | 57095408 | 57132139 | − | 36,731 | 57,063,773 | 57,163,774 | 100,001 | 2 |
| TAF15 | chr17 | 35713791 | 35864615 | + | 150,824 | 35,713,791 | 35,864,615 | 150,824 | 2 |
| TCF12 | chr15 | 56918623 | 57299281 | + | 380,658 | 56,918,623 | 57,299,281 | 380,658 | 2 |
| TFE3 | chrX | 49028726 | 49043410 | − | 14,684 | 48,986,067 | 49,086,069 | 100,002 | 2 |
| TFG | chr3 | 100709295 | 100748964 | + | 39,669 | 100,679,129 | 100,779,130 | 100,001 | 2 |
| THBS1 | chr15 | 39581079 | 39599466 | + | 18,387 | 39,540,272 | 39,640,273 | 100,001 | 2 |
| TSC1 | chr9 | 132891348 | 132946874 | − | 55,526 | 132,869,110 | 132,969,112 | 100,002 | 2 |
| TSC2 | chr16 | 2047967 | 2089491 | + | 41,524 | 2,018,728 | 2,118,730 | 100,002 | 2 |
| USP6 | chr17 | 5116032 | 5175034 | + | 59,002 | 5,095,532 | 5,195,534 | 100,002 | 2 |
| VEGFA | chr6 | 43770184 | 43786487 | + | 16,303 | 43,728,335 | 43,828,336 | 100,001 | 2 |
| VHL | chr3 | 10141778 | 10153667 | + | 11,889 | 10,097,722 | 10,197,723 | 100,001 | 2 |
| YAP1 | chr11 | 102110447 | 102233424 | + | 122,977 | 102,110,447 | 102,233,424 | 122,977 | 2 |
| YWHAE | chr17 | 1344275 | 1400222 | − | 55,947 | 1,322,248 | 1,422,249 | 100,001 | 2 |

[symbol]

indicates data missing or illegible when filed

What is claimed is:

1. A kit, comprising:
a plurality of oligonucleotide probes capable of hybridizing to target nucleic acid fragments of cancer gene exons, introns, promoters and external regions surrounding cancer genes, resulting from fragmentation of a nucleic acid sample, wherein:
a plurality of oligonucleotide probes capable of hybridizing to external regions comprises (i) a set of probe pairs each capable of hybridizing to a target nucleic acid fragment of about 260 consecutive nucleotides or longer, and (ii) a set of single oligonucleotide probes each capable of hybridizing to a nucleic acid fragment of about 130 consecutive nucleotides to about 260 consecutive nucleotides in length;
the probes in the set of oligonucleotide probe pairs and the set of single oligonucleotide probes comprise a polynucleotide (i) 120 consecutive nucleotides in length, (ii) 100% complementary to a subsequence of a nucleic acid fragment, (iii) containing an average GC content of about 40 percent to about 60 percent, and (iv) complementary to a subsequence of a nucleic acid that does not repeat in the nucleic acid fragments;
each of the probe pairs in the set of probe pairs comprises (i) a first oligonucleotide probe comprising a 5′ end 5 consecutive nucleotides from the 5′ end of the fragment, and (ii) a second oligonucleotide probe comprising a 3′ end 5 consecutive nucleotides from the 3′ end of the nucleic acid fragment to which the first oligonucleotide probe of the probe pair is capable of hybridizing; and
each probe in the set of single oligonucleotide probes comprises a 5′ or 3′ end 5 consecutive nucleotides from the 5′ end of a nucleic acid fragment.

2. A composition, comprising: a plurality of oligonucleotide probes capable of hybridizing to target nucleic acid fragments of cancer gene exons, introns, promoters, and external regions surrounding cancer genes resulting from nucleic acid fragmentation of a nucleic acid sample, wherein:
a plurality of external region directed oligonucleotide probes capable of hybridizing to target nucleic acid fragments from external regions comprises (i) a set of probe pairs each capable of hybridizing to a target nucleic acid fragment of about 260 consecutive nucleotides or longer, and (ii) a set of single oligonucleotide probes each capable of hybridizing to a nucleic acid fragment of about 130 consecutive nucleotides to about 260 consecutive nucleotides in length;
the external region directed oligonucleotide probes in the set of oligonucleotide probe pairs and the set of single oligonucleotide probes comprise a polynucleotide (i) about 110 to about 130 consecutive nucleotides in length, (ii) substantially complementary to a subsequence of a nucleic acid fragment, (iii) containing an average GC content of about 40 percent to about 60 percent, and (iv) complementary to a subsequence of a nucleic acid that does not repeat in the nucleic acid fragments;
each of the oligonucleotide probe pairs in the set of oligonucleotide probe pairs comprises (i) a first oligonucleotide probe comprising a 5′ end about 2 to about 15 consecutive nucleotides from the 5′ end of the fragment, and (ii) a second oligonucleotide probe comprising a 3′ end about 2 to about 15 consecutive nucleotides from the 3′ end of the nucleic acid fragment to which the first oligonucleotide probe of the probe pair is capable of hybridizing; and
each oligonucleotide probe in the set of single oligonucleotide probes comprises a 5′ or 3′ end about 2 to about 15 consecutive nucleotides from the 5′ end of a nucleic acid fragment.

3. A method for nucleic acid enrichment, comprising:
subjecting target nucleic acid from a nucleic acid sample to nucleic acid cleavage conditions in which nucleic acid fragments are generated;
subjecting the target nucleic acid fragments to linking conditions in which proximity ligated nucleic acid molecules are generated;
contacting the proximity ligated nucleic acid molecules (or amplified copies thereof) with a composition comprising a plurality of oligonucleotide probes under hybridization conditions in which hybridization complexes comprising proximity ligated nucleic acid hybridized to oligonucleotide probes are generated;
isolating the complexes;
analyzing nucleic acid in the complexes, and;
wherein plurality of oligonucleotide probes capable of hybridizing to target nucleic acid fragments of cancer gene exons, introns, promoters, and external regions surrounding cancer genes resulting from nucleic acid fragmentation of a nucleic acid sample comprise
a plurality of external region directed oligonucleotide probes capable of hybridizing to target nucleic acid fragments from external regions comprises (i) a set of probe pairs each capable of hybridizing to a target nucleic acid fragment of about 260 consecutive nucleotides or longer, and (ii) a set of single oligonucleotide probes each capable of hybridizing to a nucleic acid fragment of about 130 consecutive nucleotides to about 260 consecutive nucleotides in length;
the external region directed oligonucleotide probes in the set of oligonucleotide probe pairs and the set of single oligonucleotide probes comprise a polynucleotide (i) about 110 to about 130 consecutive nucleotides in length, (ii) substantially complementary to a subsequence of a nucleic acid fragment, (iii) containing an average GC content of about 40 percent to about 60 percent, and (iv) complementary to a subsequence of a nucleic acid that does not repeat in the nucleic acid fragments;
each of the oligonucleotide probe pairs in the set of oligonucleotide probe pairs comprises (i) a first oligonucleotide probe comprising a 5′ end about 2 to about 15 consecutive nucleotides from the 5′ end of the fragment, and (ii) a second oligonucleotide probe comprising a 3′ end about 2 to about 15 consecutive nucleotides from the 3′ end of the nucleic acid fragment to which the first oligonucleotide probe of the probe pair is capable of hybridizing; and
each oligonucleotide probe in the set of single oligonucleotide probes comprises a 5′ or 3′ end about 2 to about 15 consecutive nucleotides from the 5′ end of a nucleic acid fragment.

4. The kit of claim 1, wherein the cancer gene is one or more gene listed in Appendix 1 or Appendix 2.

5. The composition of claim 2, wherein the cancer gene is one or more gene listed in Appendix 1 or Appendix 2.

6. The method of claim 3, wherein the cancer gene is one or more gene listed in Appendix 1 or Appendix 2.

7. The kit of claim 1, wherein the oligonucleotide probes are capable of hybridizing to target nucleic acid fragments of external regions surrounding cancer genes.

8. The composition of claim 2, wherein the oligonucleotide probes are capable of hybridizing to target nucleic acid fragments of external regions surrounding cancer genes.

9. The method of claim 3, wherein the oligonucleotide probes are capable of hybridizing to target nucleic acid fragments of external regions surrounding cancer genes.

10. The kit of claim 1, wherein the oligonucleotide probes comprise a capture agent and the solid phase comprises a capture agent counterpart suitable for binding to a capture agent of the oligonucleotide probes.

11. The composition of claim 2, wherein the oligonucleotide probes comprise a capture agent and the solid phase comprises a capture agent counterpart suitable for binding to a capture agent of the oligonucleotide probes.

12. The method of claim 3, wherein the oligonucleotide probes comprise a capture agent and the solid phase comprises a capture agent counterpart suitable for binding to a capture agent of the oligonucleotide probes.

13. The kit of claim 10, wherein the capture agent and the capture agent counterpart independently are selected from biotin, avidin and streptavidin.

14. The method of claim 12, wherein the capture agent and the capture agent counterpart independently are selected from biotin, avidin and streptavidin.
